SEQUENCE LISTING (208 sequences)

SEQ ID NO: 1 - protein, 5 aa, Homo sapiens
  REGION 1..5: note = Description of sequence: CPP motif sequence
  MOD_RES 2: note = Naphthylalanine
FXRRR

SEQ ID NO: 2 - protein, 6 aa, Homo sapiens
  REGION 1..6: note = Description of sequence: CPP motif sequence
  MOD_RES 2: note = Naphthylalanine
FXRRRC

SEQ ID NO: 3 - protein, 6 aa, Homo sapiens
  REGION 1..6: note = Description of sequence: CPP motif sequence
  MOD_RES 2: note = Naphthylalanine
  MOD_RES 6: note = Selenocysteine
FXRRRX

SEQ ID NO: 4 - protein, 5 aa, Homo sapiens
  REGION 1..5: note = Description of sequence: CPP motif sequence
  MOD_RES 4: note = Naphthylalanine
RRRXF

SEQ ID NO: 5 - protein, 6 aa, Homo sapiens
  REGION 1..6: note = Description of sequence: CPP motif sequence
  MOD_RES 5: note = Naphthylalanine
RRRRXF

SEQ ID NO: 6 - protein, 6 aa, Homo sapiens
  REGION 1..6: note = Description of sequence: CPP motif sequence
  MOD_RES 2: note = Naphthylalanine
FXRRRR

SEQ ID NO: 7 - protein, 6 aa, Homo sapiens
  REGION 1..6: note = Description of sequence: CPP motif sequence
  MOD_RES 2: note = D-Naphthylalanine
  MOD_RES 3: note = D-Arginine
  MOD_RES 5: note = D-Arginine
FXRRRR

SEQ ID NO: 8 - protein, 6 aa, Homo sapiens
  REGION 1..6: note = Description of sequence: CPP motif sequence
  MOD_RES 2: note = D-Naphthylalanine
  MOD_RES 3: note = D-Arginine
  MOD_RES 5: note = D-Arginine
FXRRRR

SEQ ID NO: 9 - protein, 6 aa, Homo sapiens
  REGION 1..6: note = Description of sequence: CPP motif sequence
  MOD_RES 2: note = Naphthylalanine
FXRRRR

SEQ ID NO: 10 - protein, 6 aa, Homo sapiens
  REGION 1..6: note = Description of sequence: CPP motif sequence
  MOD_RES 1: note = D-phenylalanine
  MOD_RES 2: note = Naphthylalanine
  MOD_RES 4: note = D-Arginine
  MOD_RES 6: note = D-Arginine
FXRRRR

SEQ ID NO: 11 - protein, 6 aa, Homo sapiens
  REGION 1..6: note = Description of sequence: CPP motif sequence
  MOD_RES 5: note = Naphthylalanine
RRFRXR

SEQ ID NO: 12 - protein, 6 aa, Homo sapiens
  REGION 1..6: note = Description of sequence: CPP motif sequence
  MOD_RES 6: note = Naphthylalanine
FRRRRX

SEQ ID NO: 13 - protein, 6 aa, Homo sapiens
  REGION 1..6: note = Description of sequence: CPP motif sequence
  MOD_RES 4: note = Naphthylalanine
  MOD_RES 1: note = D-Arginine
RRFRXR

SEQ ID NO: 14 - protein, 6 aa, Homo sapiens
  REGION 1..6: note = Description of sequence: CPP motif sequence
  MOD_RES 3: note = Naphthylalanine
RRXFRR

SEQ ID NO: 15 - protein, 7 aa, Homo sapiens
  REGION 1..7: note = Description of sequence: CPP motif sequence
CRRRRFW

SEQ ID NO: 16 - protein, 7 aa, Homo sapiens
  REGION 1..7: note = Description of sequence: CPP motif sequence
  MOD_RES 2: note = D-phenylalanine
  MOD_RES 3: note = Naphthylalanine
  MOD_RES 4: note = D-Arginine
  MOD_RES 6: note = D-Arginine
FFXRRRR

SEQ ID NO: 17 - protein, 7 aa, Homo sapiens
  REGION 1..7: note = Description of sequence: CPP motif sequence
  MOD_RES 3: note = Naphthylalanine
FFXRRRR

SEQ ID NO: 18 - protein, 7 aa, Homo sapiens
  REGION 1..7: note = Description of sequence: CPP motif sequence
  MOD_RES 6: note = Naphthylalanine
RFRFRXR

SEQ ID NO: 19 - protein, 7 aa, Homo sapiens
  REGION 1..7: note = Description of sequence: CPP motif sequence
  MOD_RES 1: note = Selenocysteine
XRRRRFW

SEQ ID NO: 20 - protein, 7 aa, Homo sapiens
  REGION 1..7: note = Description of sequence: CPP motif sequence
CRRRRFW

SEQ ID NO: 21 - protein, 8 aa, Homo sapiens
  REGION 1..8: note = Description of sequence: CPP motif sequence
  MOD_RES 2: note = Naphthylalanine
FXRRRRQK

SEQ ID NO: 22 - protein, 8 aa, Homo sapiens
  REGION 1..8: note = Description of sequence: CPP motif sequence
  MOD_RES 2: note = Naphthylalanine
FXRRRRQC

SEQ ID NO: 23 - protein, 7 aa, Homo sapiens
  REGION 1..7: note = Description of sequence: CPP motif sequence
  MOD_RES 2: note = D-phenylalanine
  MOD_RES 2: note = Naphthylalanine
  MOD_RES 4: note = D-Arginine
  MOD_RES 6: note = D-Arginine
FXRRRRR

SEQ ID NO: 24 - protein, 7 aa, Homo sapiens
  REGION 1..7: note = Description of sequence: CPP motif sequence
  MOD_RES 2: note = Naphthylalanine
FXRRRRR

SEQ ID NO: 25 - protein, 9 aa, Homo sapiens
  REGION 1..9: note = Description of sequence: CPP motif sequence
  MOD_RES 5: note = Naphthylalanine
  MOD_RES 8: note = Nle
RRRRXFDXC

SEQ ID NO: 26 - protein, 5 aa, Homo sapiens
  REGION 1..5: note = Description of sequence: CPP motif sequence
  MOD_RES 2: note = Naphthylalanine
FXRRR

SEQ ID NO: 27 - protein, 5 aa, Homo sapiens
  REGION 1..5: note = Description of sequence: CPP motif sequence
FWRRR

SEQ ID NO: 28 - protein, 5 aa, Homo sapiens
  REGION 1..5: note = Description of sequence: CPP motif sequence
  MOD_RES 4: note = Naphthylalanine
RRRXF

SEQ ID NO: 29 - protein, 5 aa, Homo sapiens
  REGION 1..5: note = Description of sequence: CPP motif sequence
RRRWF

SEQ ID NO: 30 - protein, 6 aa, Homo sapiens
  REGION 1..6: note = Description of sequence: CPP motif sequence
  MOD_RES 2: note = Naphthylalanine
FXRRRR

SEQ ID NO: 31 - protein, 5 aa, Homo sapiens
  REGION 1..5: note = Description of sequence: CPP motif sequence
FFRRR

SEQ ID NO: 32 - protein, 5 aa, Homo sapiens
  REGION 1..5: note = Description of sequence: CPP motif sequence
  MOD_RES 3: note = D-Arginine
  MOD_RES 5: note = D-Arginine
FFRRR

SEQ ID NO: 33 - protein, 5 aa, Homo sapiens
  REGION 1..5: note = Description of sequence: CPP motif sequence
  MOD_RES 4: note = D-Arginine
FFRRR

SEQ ID NO: 34 - protein, 5 aa, Homo sapiens
  REGION 1..5: note = Description of sequence: CPP motif sequence
FRFRR

SEQ ID NO: 35 - protein, 5 aa, Homo sapiens
  REGION 1..5: note = Description of sequence: CPP motif sequence
FRRFR

SEQ ID NO: 36 - protein, 5 aa, Homo sapiens
  REGION 1..5: note = Description of sequence: CPP motif sequence
FRRRF

SEQ ID NO: 37 - protein, 5 aa, Homo sapiens
  REGION 1..5: note = Description of sequence: CPP motif sequence
  MOD_RES 2: note = Naphthylalanine
GXRRR

SEQ ID NO: 38 - protein, 5 aa, Homo sapiens
  REGION 1..5: note = Description of sequence: CPP motif sequence
FFFRA

SEQ ID NO: 39 - protein, 5 aa, Homo sapiens
  REGION 1..5: note = Description of sequence: CPP motif sequence
FFFRR

SEQ ID NO: 40 - protein, 6 aa, Homo sapiens
  REGION 1..6: note = Description of sequence: CPP motif sequence
FFRRRR

SEQ ID NO: 41 - protein, 6 aa, Homo sapiens
  REGION 1..6: note = Description of sequence: CPP motif sequence
FRRFRR

SEQ ID NO: 42 - protein, 6 aa, Homo sapiens
  REGION 1..6: note = Description of sequence: CPP motif sequence
FRRRFR

SEQ ID NO: 43 - protein, 6 aa, Homo sapiens
  REGION 1..6: note = Description of sequence: CPP motif sequence
RFFRRR

SEQ ID NO: 44 - protein, 6 aa, Homo sapiens
  REGION 1..6: note = Description of sequence: CPP motif sequence
RFRRFR

SEQ ID NO: 45 - protein, 6 aa, Homo sapiens
  REGION 1..6: note = Description of sequence: CPP motif sequence
FRFRRR

SEQ ID NO: 46 - protein, 6 aa, Homo sapiens
  REGION 1..6: note = Description of sequence: CPP motif sequence
FFFRRR

SEQ ID NO: 47 - protein, 6 aa, Homo sapiens
  REGION 1..6: note = Description of sequence: CPP motif sequence
FFRRRF

SEQ ID NO: 48 - protein, 6 aa, Homo sapiens
  REGION 1..6: note = Description of sequence: CPP motif sequence
FRFFRR

SEQ ID NO: 49 - protein, 6 aa, Homo sapiens
  REGION 1..6: note = Description of sequence: CPP motif sequence
RRFFFR

SEQ ID NO: 50 - protein, 6 aa, Homo sapiens
  REGION 1..6: note = Description of sequence: CPP motif sequence
FFRFRR

SEQ ID NO: 51 - protein, 6 aa, Homo sapiens
  REGION 1..6: note = Description of sequence: CPP motif sequence
FFRRFR

SEQ ID NO: 52 - protein, 6 aa, Homo sapiens
  REGION 1..6: note = Description of sequence: CPP motif sequence
FRRFFR

SEQ ID NO: 53 - protein, 6 aa, Homo sapiens
  REGION 1..6: note = Description of sequence: CPP motif sequence
FRRFRF

SEQ ID NO: 54 - protein, 6 aa, Homo sapiens
  REGION 1..6: note = Description of sequence: CPP motif sequence
FRFRFR

SEQ ID NO: 55 - protein, 6 aa, Homo sapiens
  REGION 1..6: note = Description of sequence: CPP motif sequence
RFFRFR

SEQ ID NO: 56 - protein, 6 aa, Homo sapiens
  REGION 1..6: note = Description of sequence: CPP motif sequence
  MOD_RES 2: note = Naphthylalanine
GXRRRR

SEQ ID NO: 57 - protein, 7 aa, Homo sapiens
  REGION 1..7: note = Description of sequence: CPP motif sequence
FFFRRRR

SEQ ID NO: 58 - protein, 7 aa, Homo sapiens
  REGION 1..7: note = Description of sequence: CPP motif sequence
RFFRRRR

SEQ ID NO: 59 - protein, 7 aa, Homo sapiens
  REGION 1..7: note = Description of sequence: CPP motif sequence
RRFFRRR

SEQ ID NO: 60 - protein, 7 aa, Homo sapiens
  REGION 1..7: note = Description of sequence: CPP motif sequence
RFFFRRR

SEQ ID NO: 61 - protein, 7 aa, Homo sapiens
  REGION 1..7: note = Description of sequence: CPP motif sequence
RRFFFRR

SEQ ID NO: 62 - protein, 7 aa, Homo sapiens
  REGION 1..7: note = Description of sequence: CPP motif sequence
FFRRFRR

SEQ ID NO: 63 - protein, 7 aa, Homo sapiens
  REGION 1..7: note = Description of sequence: CPP motif sequence
FFRRRRF

SEQ ID NO: 64 - protein, 7 aa, Homo sapiens
  REGION 1..7: note = Description of sequence: CPP motif sequence
FRRFFRR

SEQ ID NO: 65 - protein, 8 aa, Homo sapiens
  REGION 1..8: note = Description of sequence: CPP motif sequence
FFFRRRRR

SEQ ID NO: 66 - protein, 9 aa, Homo sapiens
  REGION 1..9: note = Description of sequence: CPP motif sequence
FFFRRRRRR

SEQ ID NO: 67 - protein, 6 aa, Homo sapiens
  REGION 1..6: note = Description of sequence: CPP motif sequence
  MOD_RES 4: note = Naphthylalanine
  MOD_RES 6: note = D-Arginine
FXRRRR

SEQ ID NO: 68 - protein, 4 aa, Homo sapiens
  REGION 1..4: note = Description of sequence: CPP motif sequence
  REGION 1..4: note = Sequence may be inserted between motif amino acid
RRRR

SEQ ID NO: 69 - protein, 6 aa, Homo sapiens
  REGION 1..6: note = Description of sequence: CPP motif sequence
  MOD_RES 2: note = D-phenylalanine
  MOD_RES 5: note = D-Arginine
FFFRRR

SEQ ID NO: 70 - protein, 6 aa, Homo sapiens
  REGION 1..6: note = Description of sequence: CPP motif sequence
  MOD_RES 1: note = D-phenylalanine
  MOD_RES 3: note = D-phenylalanine
  MOD_RES 4: note = D-Arginine
  MOD_RES 6: note = D-Arginine
FFFRRR

SEQ ID NO: 71 - protein, 6 aa, Homo sapiens
  REGION 1..6: note = Description of sequence: CPP motif sequence
  MOD_RES 1: note = D-phenylalanine
  MOD_RES 3: note = D-phenylalanine
  MOD_RES 4: note = D-Arginine
FFFRRR

SEQ ID NO: 72 - protein, 6 aa, Homo sapiens
  REGION 1..6: note = Description of sequence: CPP motif sequence
  MOD_RES 2: note = D-phenylalanine
  MOD_RES 4: note = D-Arginine
  MOD_RES 6: note = D-Arginine
FFFRRR

SEQ ID NO: 73 - protein, 6 aa, Homo sapiens
  REGION 1..6: note = Description of sequence: CPP motif sequence
  MOD_RES 1: note = D-naphthylalanine
  MOD_RES 3: note = D-Naphthylalanine
  MOD_RES 4: note = D-Arginine
  MOD_RES 6: note = D-Arginine
FFXRRR

SEQ ID NO: 74 - protein, 6 aa, Homo sapiens
  REGION 1..6: note = Description of sequence: CPP motif sequence
  MOD_RES 3: note = D-phenylalanine
  MOD_RES 2: note = Naphthylalanine
  MOD_RES 4: note = D-Arginine
  MOD_RES 6: note = D-Arginine
FXFRRR

SEQ ID NO: 75 - protein, 6 aa, Homo sapiens
  REGION 1..6: note = Description of sequence: CPP motif sequence
  MOD_RES 3: note = D-naphthylalanine
  MOD_RES 1: note = D-Naphthylalanine
  MOD_RES 3: note = D-Naphthylalanine
  MOD_RES 4: note = D-Arginine
  MOD_RES 6: note = D-Arginine
XFFRRR

SEQ ID NO: 76 - protein, 5 aa, Homo sapiens
  REGION 1..5: note = Description of sequence: CPP motif sequence
  MOD_RES 2: note = Naphthylalanine
  MOD_RES 3: note = D-Arginine
  MOD_RES 5: note = D-Arginine
FXRRR

SEQ ID NO: 77 - protein, 5 aa, Homo sapiens
  REGION 1..5: note = Description of sequence: CPP motif sequence
  MOD_RES 1: note = D-phenylalanine
  MOD_RES 2: note = Naphthylalanine
  MOD_RES 3: note = D-Arginine
  MOD_RES 5: note = D-Arginine
FXRRR

SEQ ID NO: 78 - protein, 8 aa, Homo sapiens
  REGION 1..8: note = Description of sequence: CPP motif sequence
  MOD_RES 2: note = D-phenylalanine
  MOD_RES 1: note = acetylated
  MOD_RES 5: note = D-Arginine
  MOD_RES 7: note = D-Arginine
KFFRRRRD

SEQ ID NO: 79 - protein, 8 aa, Homo sapiens
  REGION 1..8: note = Description of sequence: CPP motif sequence
  MOD_RES 2: note = D-phenylalanine
  MOD_RES 1: note = acetylated
  MOD_RES 1: note = 2,3-diaminopropionic acid
  MOD_RES 5: note = D-Arginine
  MOD_RES 7: note = D-Arginine
XFFRRRRD

SEQ ID NO: 80 - protein, 7 aa, Homo sapiens
  REGION 1..7: note = Description of sequence: CPP motif sequence
  MOD_RES 1: note = Homoproline
  MOD_RES 2: note = Naphthylalanine
  MOD_RES 5: note = D-Arginine
  MOD_RES 6: note = D-Arginine
XXRERRE

SEQ ID NO: 81 - protein, 7 aa, Homo sapiens
  REGION 1..7: note = Description of sequence: CPP motif sequence
  MOD_RES 1: note = Homoproline
  MOD_RES 2: note = Naphthylalanine
  MOD_RES 5: note = D-Arginine
  MOD_RES 6: note = D-Arginine
XXRRRRE

SEQ ID NO: 82 - protein, 7 aa, Homo sapiens
  REGION 1..7: note = Description of sequence: CPP motif sequence
  MOD_RES 1: note = Homoproline
  MOD_RES 2..3: note = Naphthylalanine
  MOD_RES 5: note = D-Arginine
  MOD_RES 6: note = D-Arginine
XXXRRRE

SEQ ID NO: 83 - protein, 7 aa, Homo sapiens
  REGION 1..7: note = Description of sequence: CPP motif sequence
  MOD_RES 1: note = Homoproline
  MOD_RES 2..3: note = Naphthylalanine
  MOD_RES 5: note = D-Arginine
  MOD_RES 6: note = D-Arginine
XXXRRRE

SEQ ID NO: 84 - protein, 7 aa, Homo sapiens
  REGION 1..7: note = Description of sequence: CPP motif sequence
  MOD_RES 1: note = Homoproline
  MOD_RES 2: note = Naphthylalanine
  MOD_RES 5: note = D-Arginine
  MOD_RES 6: note = D-Arginine
XXFRRRE

SEQ ID NO: 85 - protein, 7 aa, Homo sapiens
  REGION 1..7: note = Description of sequence: CPP motif sequence
  MOD_RES 1: note = Homoproline
  MOD_RES 2: note = Naphthylalanine
  MOD_RES 5: note = D-Arginine
  MOD_RES 6: note = D-Arginine
XXFRRRE

SEQ ID NO: 86 - protein, 7 aa, Homo sapiens
  REGION 1..7: note = Description of sequence: CPP motif sequence
  MOD_RES 1: note = Homoproline
  MOD_RES 2: note = Naphthylalanine
  MOD_RES 5: note = D-Arginine
  MOD_RES 6: note = D-Arginine
XXFRRRE

SEQ ID NO: 87 - protein, 7 aa, Homo sapiens
  REGION 1..7: note = Description of sequence: CPP motif sequence
  MOD_RES 1: note = Homoproline
  MOD_RES 2: note = Naphthylalanine
  MOD_RES 5: note = D-Arginine
  MOD_RES 6: note = D-Arginine
XXFRRRE

SEQ ID NO: 88 - protein, 7 aa, Homo sapiens
  REGION 1..7: note = Description of sequence: CPP motif sequence
  MOD_RES 1: note = Homoproline
  MOD_RES 2..3: note = Naphthylalanine
  MOD_RES 5: note = D-Arginine
  MOD_RES 6: note = D-Arginine
XXXRRRE

SEQ ID NO: 89 - protein, 7 aa, Homo sapiens
  REGION 1..7: note = Description of sequence: CPP motif sequence
  MOD_RES 1: note = Homoproline
  MOD_RES 2..3: note = Naphthylalanine
  MOD_RES 5: note = D-Arginine
  MOD_RES 6: note = D-Arginine
XXXRRRE

SEQ ID NO: 90 - protein, 10 aa, Homo sapiens
  REGION 1..10: note = Description of sequence: CPP motif sequence
  MOD_RES 3: note = D-Arginine
  MOD_RES 2: note = Cyclohexylalanine
  MOD_RES 5: note = D-Arginine
  MOD_RES 4: note = Cyclohexylalanine
  MOD_RES 6: note = Cyclohexylalanine
  MOD_RES 7: note = D-Arginine
  MOD_RES 6: note = Cyclohexylalanine
  SITE 1..10: note = Macrocyclization by multicomponent reaction with aziridine aldehyde and isocyanide
PXRXRXRXRG

SEQ ID NO: 91 - protein, 14 aa, Homo sapiens
  REGION 1..14: note = Description of sequence: CPP motif sequence
  MOD_RES 1: note = Trimesic acid
  MOD_RES 3: note = D-valine Modification
  MOD_RES 5: note = 4-(phosphonodifluoromethyl)phenylalanine
  MOD_RES 7: note = 2,3-diaminopropionic acid
  MOD_RES 9: note = Naphthylalanine
  MOD_RES 14: note = 2,3-diaminopropionic acid
  CROSSLNK 1..14: note = cyclic bond
  CROSSLNK 7..14: note = cyclic bond
XSVPXHXFXRRRRX

SEQ ID NO: 92 - protein, 18 aa, Homo sapiens
  REGION 1..18: note = Description of sequence: CPP motif sequence
  MOD_RES 1: note = Trimesic acid
  MOD_RES 2: note = D-alanine
  MOD_RES 3: note = MeGly
  MOD_RES 5: note = phosphorylated D-threonine
  MOD_RES 6: note = Homoproline
  MOD_RES 7: note = Naphthylalanine
  MOD_RES 9: note = D-alanine
  MOD_RES 10: note = 2,3-diaminopropionic acid
  MOD_RES 12: note = Naphthylalanine
  MOD_RES 18: note = 2,3-diaminopropionic acid
XAXDXXXRAAXFXRRRRX

SEQ ID NO: 93 - protein, 16 aa, Homo sapiens
  REGION 1..16: note = Description of sequence: CPP motif sequence
  SITE 1..16: note = Three Cys side chains bicyclized with tris(bromomethyl)benzene
CRRSRRGCGRRSRRCG

SEQ ID NO: 94 - protein, 6 aa, Homo sapiens
  REGION 1..6: note = Description of sequence: CPP motif sequence
  MOD_RES 2: note = dodecanoyl
KXRRRR

SEQ ID NO: 95 - protein, 16 aa, Homo sapiens
  REGION 1..16: note = Description of sequence: CPP motif sequence
LKKLCKLLKKLCKLAG

SEQ ID NO: 96 - protein, 9 aa, Homo sapiens
  REGION 1..9: note = Description of sequence: CPP motif sequence
  CROSSLNK 5..9: note = Cyclization between Lys and Glu
RRRRKRRRE

SEQ ID NO: 97 - protein, 9 aa, Homo sapiens
  REGION 1..9: note = Description of sequence: CPP motif sequence
  CROSSLNK 4..9: note = Cyclization between Lys and Glu
RRRKRRRRE

SEQ ID NO: 98 - protein, 9 aa, Homo sapiens
  REGION 1..9: note = Description of sequence: CPP motif sequence
  CROSSLNK 3..9: note = Cyclization between Lys and Glu
RRKRRRRRE

SEQ ID NO: 99 - protein, 9 aa, Homo sapiens
  REGION 1..9: note = Description of sequence: CPP motif sequence
  CROSSLNK 2..9: note = Cyclization between Lys and Glu
RKRRRRRRE

SEQ ID NO: 100 - protein, 8 aa, Homo sapiens
  REGION 1..8: note = Description of sequence: CPP motif sequence
CRCRCRCR

SEQ ID NO: 101 - protein, 12 aa, Homo sapiens
  REGION 1..12: note = Description of sequence: CPP motif sequence
  MOD_RES 1: note = L-propargylglycine
  MOD_RES 12: note = L-6-Azido-2-amino-hexanoic
  CROSSLNK 1..12: note = Cyclization by the click reaction between Pra and Azk
XLRKRLRKFRNX

SEQ ID NO: 102 - protein, 9 aa, Homo sapiens
  REGION 1..9: note = Description of sequence: CPP motif sequence
  MOD_RES 2..4: note = 2,3-diaminopropionic acid
  MOD_RES 7..8: note = 2,3-diaminopropionic acid
TXXXFLXXT

SEQ ID NO: 103 - protein, 9 aa, Homo sapiens
  REGION 1..9: note = Description of sequence: CPP motif sequence
  MOD_RES 2: note = L-2-amino-3-guanidinylpropionic acid
  MOD_RES 3: note = 2,3-diaminopropionic acid
  MOD_RES 4: note = L-2-amino-3-guanidinylpropionic acid
  MOD_RES 7..8: note = L-2-amino-3-guanidinylpropionic acid
TXXXFLXXT

SEQ ID NO: 104 - protein, 6 aa, Homo sapiens
  REGION 1..6: note = Description of sequence: Motif
RRRWWW

SEQ ID NO: 105 - protein, 6 aa, Homo sapiens
  REGION 1..6: note = Description of sequence: Motif
WWWRRR

SEQ ID NO: 106 - protein, 6 aa, Homo sapiens
  REGION 1..6: note = Description of sequence: Motif
GDSPAS

SEQ ID NO: 107 - protein, 9 aa, Homo sapiens
  REGION 1..9: note = Description of sequence: Motif
GGSGGSGGS

SEQ ID NO: 108 - protein, 13 aa, Homo sapiens
  REGION 1..13: note = Description of sequence: Loop Sequence
TGRGDSPASSKPI

SEQ ID NO: 109 - protein, 9 aa, Homo sapiens
  REGION 1..9: note = Description of sequence: Loop Sequence
APAVTVRYY

SEQ ID NO: 110 - protein, 8 aa, Homo sapiens
  REGION 1..8: note = Description of sequence: Loop Sequence
GGNSPVQE

SEQ ID NO: 111 - protein, 13 aa, Homo sapiens
  REGION 1..13: note = Description of sequence: Loop Sequence
TGRRRRWWWSKPI

SEQ ID NO: 112 - protein, 11 aa, Homo sapiens
  REGION 1..11: note = Description of sequence: Loop Sequence
APWWWRRRRYY

SEQ ID NO: 113 - protein, 12 aa, Homo sapiens
  REGION 1..12: note = Description of sequence: Loop Sequence
GGRRRRWWWVQE

SEQ ID NO: 114 - protein, 9 aa, Homo sapiens
  REGION 1..9: note = Description of sequence: Loop Sequence
APAWYWRYY

SEQ ID NO: 115 - protein, 10 aa, Homo sapiens
  REGION 1..10: note = Description of sequence: Loop Sequence
TGRRRRSKPI

SEQ ID NO: 116 - protein, 9 aa, Homo sapiens
  REGION 1..9: note = Description of sequence: Loop Sequence
APARRRRYY

SEQ ID NO: 117 - protein, 10 aa, Homo sapiens
  REGION 1..10: note = Description of sequence: Loop Sequence
TGWYWRSKPI

SEQ ID NO: 118 - protein, 127 aa, Homo sapiens
  REGION 1..127: note = Description of sequence: Fusion protein
MGSSHHHHHHSSGLVPRGSHMVSDVPRDLEVVAATPTSLLISWDAPAVTVRYYRITYGETGGNSPVQEFTVPGSKSTATISGLKPGVDYTITVYAVTGRGDSPASSKPISINYRTGGSGGSGGSELC

SEQ ID NO: 119 - protein, 127 aa, Homo sapiens
  REGION 1..127: note = Description of sequence: Fusion protein
MGSSHHHHHHSSGLVPRGSHMVSDVPRDLEVVAATPTSLLISWDAPAVTVRYYRITYGETGGNSPVQEFTVPGSKSTATISGLKPGVDYTITVYAVTGRRRRWWWSKPISINYRTGGSGGSGGSELC

SEQ ID NO: 120 - protein, 129 aa, Homo sapiens
  REGION 1..129: note = Description of sequence: Fusion protein
MGSSHHHHHHSSGLVPRGSHMVSDVPRDLEVVAATPTSLLISWDAPWWWRRRRYYRITYGETGGNSPVQEFTVPGSKSTATISGLKPGVDYTITVYAVTGRGDSPASSKPISINYRTGGSGGSGGSELC

SEQ ID NO: 121 - protein, 131 aa, Homo sapiens
  REGION 1..131: note = Description of sequence: Fusion protein
MGSSHHHHHHSSGLVPRGSHMVSDVPRDLEVVAATPTSLLISWDAPAVTVRYYRITYGETGGRRRRWWWVQEFTVPGSKSTATISGLKPGVDYTITVYAVTGRGDSPASSKPISINYRTGGSGGSGGSELC

SEQ ID NO: 122 - protein, 124 aa, Homo sapiens
  REGION 1..124: note = Description of sequence: Fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELC

SEQ ID NO: 123 - protein, 127 aa, Homo sapiens
  REGION 1..127: note = Description of sequence: Fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRGDSPASSKPISINYRTGGSGGSGGSELC

SEQ ID NO: 124 - protein, 124 aa, Homo sapiens
  REGION 1..124: note = Description of sequence: Fusion protein
MGSSHHHHHHSSGLVPRGSHMVSDVPRDLEVVAATPTSLLISWDAPAVTVRYYRITYGETGGNSPVQEFTVPGSKSTATISGLKPGVDYTITVYAVTGRRRRSKPISINYRTGGSGGSGGSELC

SEQ ID NO: 125 - protein, 124 aa, Homo sapiens
  REGION 1..124: note = Description of sequence: Fusion protein
MGSSHHHHHHSSGLVPRGSHMVSDVPRDLEVVAATPTSLLISWDAPARRRRYYRITYGETGGNSPVQEFTVPGSKSTATISGLKPGVDYTITVYAVTGWYWRSKPISINYRTGGSGGSGGSELC

SEQ ID NO: 126 - protein, 444 aa, Homo sapiens
  REGION 1..444: note = Description of sequence: Fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELMEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHN

SEQ ID NO: 127 - protein, 220 aa, Homo sapiens
  REGION 1..220: note = Description of sequence: Fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELGSVSSVPTKLEVVAATPTSLLISWDAPAVTVDYYVITYGETGGNSPVQKFEVPGSKSTATISGLKPGVDYTITVYAWGWHGQVYYYMGSPISINYRT

SEQ ID NO: 128 - protein, 225 aa, Homo sapiens
  REGION 1..225: note = Description of sequence: Fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELKTSNTIRVLLPNQEWTVVKVRNGMSLHDSLMKALKRHGLQPESSAVFRLLHEHKGKKARLDWNTDAASLIGEELQVDFLDHVPLTTHNFARKTFLKLGIHRD

SEQ ID NO: 129 - protein, 5 aa, Homo sapiens
  REGION 1..5: note = Description of sequence: Loop Sequence
AVTVR

SEQ ID NO: 130 - protein, 7 aa, Homo sapiens
  REGION 1..7: note = Description of sequence: Loop Sequence
GGNSPVQ

SEQ ID NO: 131 - protein, 4 aa, Homo sapiens
  REGION 1..4: note = Description of sequence: Loop Sequence
PGSK

SEQ ID NO: 132 - protein, 8 aa, Homo sapiens
  REGION 1..8: note = Description of sequence: Loop Sequence
GRGDSPAS

SEQ ID NO: 133 - protein, 8 aa, Homo sapiens
  REGION 1..8: note = Description of sequence: Loop Sequence
RRRRWWWW

SEQ ID NO: 134 - protein, 9 aa, Homo sapiens
  REGION 1..9: note = Description of sequence: Loop Sequence
APAYWWRYY

SEQ ID NO: 135 - protein, 10 aa, Homo sapiens
  REGION 1..10: note = Description of sequence: Loop Sequence
TGRRRRSKPI

SEQ ID NO: 136 - protein, 9 aa, Homo sapiens
  REGION 1..9: note = Description of sequence: Loop Sequence
APAWWARYY

SEQ ID NO: 137 - protein, 10 aa, Homo sapiens
  REGION 1..10: note = Description of sequence: Loop Sequence
TGRRRRSKPI

SEQ ID NO: 138 - protein, 10 aa, Homo sapiens
  REGION 1..10: note = Description of sequence: Loop Sequence
APAWWWRRYY

SEQ ID NO: 139 - protein, 10 aa, Homo sapiens
  REGION 1..10: note = Description of sequence: Loop Sequence
TGRRASSKPI

SEQ ID NO: 140 - protein, 9 aa, Homo sapiens
  REGION 1..9: note = Description of sequence: Loop Sequence
APAWWARYY

SEQ ID NO: 141 - protein, 11 aa, Homo sapiens
  REGION 1..11: note = Description of sequence: Loop Sequence
TGWRRRRSKPI

SEQ ID NO: 142 - protein, 9 aa, Homo sapiens
  REGION 1..9: note = Description of sequence: Loop Sequence
APAWWRRYY

SEQ ID NO: 143 - protein, 124 aa, Homo sapiens
  REGION 1..124: note = Description of sequence: Fusion protein
MGSSHHHHHHSSGLVPRGSHMVSDVPRDLEVVAATPTSLLISWDAPAYWWRYYRITYGETGGNSPVQEFTVPGSKSTATISGLKPGVDYTITVYAVTGRRRRSKPISINYRTGGSGGSGGSELC

SEQ ID NO: 144 - protein, 124 aa, Homo sapiens
  REGION 1..124: note = Description of sequence: Fusion protein
MGSSHHHHHHSSGLVPRGSHMVSDVPRDLEVVAATPTSLLISWDAPAWWARYYRITYGETGGNSPVQEFTVPGSKSTATISGLKPGVDYTITVYAVTGRRRRSKPISINYRTGGSGGSGGSELC

SEQ ID NO: 145 - protein, 125 aa, Homo sapiens
  REGION 1..125: note = Description of sequence: Fusion protein
MGSSHHHHHHSSGLVPRGSHMVSDVPRDLEVVAATPTSLLISWDAPAWWWRRYYRITYGETGGNSPVQEFTVPGSKSTATISGLKPGVDYTITVYAVTGRRASSKPISINYRTGGSGGSGGSELC

SEQ ID NO: 146 - protein, 125 aa, Homo sapiens
  REGION 1..125: note = Description of sequence: Fusion protein
MGSSHHHHHHSSGLVPRGSHMVSDVPRDLEVVAATPTSLLISWDAPAWWARYYRITYGETGGNSPVQEFTVPGSKSTATISGLKPGVDYTITVYAVTGWRRRRSKPISINYRTGGSGGSGGSELC

SEQ ID NO: 147 - protein, 124 aa, Homo sapiens
  REGION 1..124: note = Description of sequence: Fusion protein
MGSSHHHHHHSSGLVPRGSHMVSDVPRDLEVVAATPTSLLISWDAPAWWRRYYRITYGETGGNSPVQEFTVPGSKSTATISGLKPGVDYTITVYAVTGRRWWSKPISINYRTGGSGGSGGSELC

SEQ ID NO: 148 - protein, 360 aa, Homo sapiens
  REGION 1..360: note = Description of sequence: Fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNDHQVYIMADKQKNGIKANFKIRHNIEDGGVQLADHYQQNTPIGDGPVLLPDNHYLFTTSTLSKDPNEKRDHMVLLEFVTAAGITHGMDELYK

SEQ ID NO: 149 - protein, 725 aa, Homo sapiens
  REGION 1..725: note = Description of sequence: Fusion protein
MGSSHHHHHHSSGLVPRGSHMVSDVPRDLEVVAATPTSLLISWDAPAWYWRYYRITYGETGGNSPVQEFTVPGSKSTATISGLKPGVDYTITVYAVTGRRRRSKPISINYRTGGSGGSGGSELMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGEQKLISEEDLGGPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 150 - protein, 10 aa, Homo sapiens
  REGION 1..10: note = Description of sequence: Loop Sequence
TGRRWWSKPI

SEQ ID NO: 151 - protein, 11 aa, Homo sapiens
  REGION 1..11: note = Description of sequence: Motif
VSGWRLFKKIS

SEQ ID NO: 152 - protein, 12 aa, Homo sapiens
  REGION 1..12: note = Description of sequence: Motif
CVSGWRLFKKIS

SEQ ID NO: 153 - protein, 4 aa, Homo sapiens
  REGION 1..4: note = Description of sequence: Motif
DLLD

SEQ ID NO: 154 - protein, 4 aa, Homo sapiens
  REGION 1..4: note = Description of sequence: Motif
LDDL

SEQ ID NO: 155 - protein, 5 aa, Homo sapiens
  REGION 1..5: note = Description of sequence: Motif
LDLLD

SEQ ID NO: 156 - protein, 5 aa, Homo sapiens
  REGION 1..5: note = Description of sequence: Motif
DLDDL

SEQ ID NO: 157 - protein, 5 aa, Homo sapiens
  REGION 1..5: note = Description of sequence: Motif
DLLDL

SEQ ID NO: 158 - protein, 5 aa, Homo sapiens
  REGION 1..5: note = Description of sequence: Motif
LDDLD

SEQ ID NO: 159 - protein, 4 aa, Homo sapiens
  REGION 1..4: note = Description of sequence: Motif
  REGION 1..4: note = May have a sequence amino acids inserted
RRRR

SEQ ID NO: 160 - protein, 5 aa, Homo sapiens
  REGION 1..5: note = Description of sequence: Motif
  REGION 1..4: note = May have a sequence amino acids inserted
RRRRR

SEQ ID NO: 161 - protein, 7 aa, Homo sapiens
  REGION 1..7: note = Description of sequence: Motif
  REGION 1..6: note = May have a sequence amino acids inserted
WWWRRRR

SEQ ID NO: 162 - protein, 6 aa, Homo sapiens
  REGION 1..6: note = Description of sequence: Example targeting moiety
  MOD_RES 2: note = Homoproline
  MOD_RES 4: note = 4-(phosphonodifluoromethyl)phenylalanine
PXGFYR

SEQ ID NO: 163 - protein, 6 aa, Homo sapiens
  REGION 1..6: note = Description of sequence: Example targeting moiety
  MOD_RES 2: note = Homoproline
  MOD_RES 4..5: note = 4-(phosphonodifluoromethyl)phenylalanine
SXIFFR

SEQ ID NO: 164 - protein, 6 aa, Homo sapiens
  REGION 1..6: note = Description of sequence: Example targeting moiety
  MOD_RES 4: note = 4-(phosphonodifluoromethyl)phenylalanine
IHIFIR

SEQ ID NO: 165 - protein, 6 aa, Homo sapiens
  REGION 1..6: note = Description of sequence: Example targeting moiety
  MOD_RES 2: note = D-alanine
  MOD_RES 5: note = Homoproline
  MOD_RES 4: note = 4-(phosphonodifluoromethyl)phenylalanine
AAIFXR

SEQ ID NO: 166 - protein, 6 aa, Homo sapiens
  REGION 1..6: note = Description of sequence: Example targeting moiety
  MOD_RES 1: note = 4-fluorophenylalanine
  MOD_RES 3: note = Homoproline
  MOD_RES 4: note = 4-(phosphonodifluoromethyl)phenylalanine
  MOD_RES 5: note = D-alanine
XSXFVR

SEQ ID NO: 167 - protein, 6 aa, Homo sapiens
  REGION 1..6: note = Description of sequence: Example targeting moiety
  MOD_RES 1: note = Homoproline
  MOD_RES 2: note = D-alanine
  MOD_RES 5: note = 4-(phosphonodifluoromethyl)phenylalanine
XNPFAR

SEQ ID NO: 168 - protein, 6 aa, Homo sapiens
  REGION 1..6: note = Description of sequence: Example targeting moiety
  MOD_RES 2: note = Phenylglycine
  MOD_RES 4: note = 4-(phosphonodifluoromethyl)phenylalanine
YXAFGR

SEQ ID NO: 169 - protein, 6 aa, Homo sapiens
  REGION 1..6: note = Description of sequence: Example targeting moiety
  MOD_RES 4: note = 4-(phosphonodifluoromethyl)phenylalanine
  MOD_RES 5: note = D-alanine
AHIFAR

SEQ ID NO: 170 - protein, 6 aa, Homo sapiens
  REGION 1..6: note = Description of sequence: Example targeting moiety
  MOD_RES 2: note = D-asparagine
  MOD_RES 4: note = 4-(phosphonodifluoromethyl)phenylalanine
  MOD_RES 5: note = D-proline
GNGFPR

SEQ ID NO: 171 - protein, 6 aa, Homo sapiens
  REGION 1..6: note = Description of sequence: Example targeting moiety
  MOD_RES 1: note = D-phenylalanine
  MOD_RES 3: note = Homoproline
  MOD_RES 4: note = 4-(phosphonodifluoromethyl)phenylalanine
FQXFIR

SEQ ID NO: 172 - protein, 6 aa, Homo sapiens
  REGION 1..6: note = Description of sequence: Example targeting moiety
  MOD_RES 4: note = 4-(phosphonodifluoromethyl)phenylalanine
SPGFHR

SEQ ID NO: 173 - protein, 6 aa, Homo sapiens
  REGION 1..6: note = Description of sequence: Example targeting moiety
  MOD_RES 1: note = Homoproline
  MOD_RES 4: note = 4-(phosphonodifluoromethyl)phenylalanine
XYIFHR

SEQ ID NO: 174 - protein, 6 aa, Homo sapiens
  REGION 1..6: note = Description of sequence: Example targeting moiety
  MOD_RES 2: note = D-valine
  MOD_RES 4: note = 4-(phosphonodifluoromethyl)phenylalanine
SVPFHR

SEQ ID NO: 175 - protein, 6 aa, Homo sapiens
  REGION 1..6: note = Description of sequence: Example targeting moiety
  MOD_RES 4: note = 4-(phosphonodifluoromethyl)phenylalanine
  MOD_RES 5: note = D-asparagine
AIPFNR

SEQ ID NO: 176 - protein, 6 aa, Homo sapiens
  REGION 1..6: note = Description of sequence: Example targeting moiety
  MOD_RES 1: note = 4-fluorophenylalanine
  MOD_RES 4: note = 4-(phosphonodifluoromethyl)phenylalanine
XSIFQR

SEQ ID NO: 177 - protein, 6 aa, Homo sapiens
  REGION 1..6: note = Description of sequence: Example targeting moiety
  MOD_RES 2: note = D-alanine
  MOD_RES 3: note = Phenylglycine
  MOD_RES 4: note = 4-(phosphonodifluoromethyl)phenylalanine
  MOD_RES 5: note = D-phenylalanine
AAXFFR

SEQ ID NO: 178 - protein, 6 aa, Homo sapiens
  REGION 1..6: note = Description of sequence: Example targeting moiety
  MOD_RES 1: note = D-asparagine
  MOD_RES 2: note = D-threonine
  MOD_RES 3: note = Phenylglycine
  MOD_RES 4: note = 4-(phosphonodifluoromethyl)phenylalanine
  MOD_RES 5: note = Phenylglycine
NTXFXR

SEQ ID NO: 179 - protein, 6 aa, Homo sapiens
  REGION 1..6: note = Description of sequence: Example targeting moiety
  MOD_RES 3: note = Phenylglycine
  MOD_RES 4: note = 4-(phosphonodifluoromethyl)phenylalanine
  MOD_RES 5: note = Nle
IPXFXR

SEQ ID NO: 180 - protein, 6 aa, Homo sapiens
  REGION 1..6: note = Description of sequence: Example targeting moiety
  MOD_RES 2: note = Homoproline
  MOD_RES 3: note = 4-fluorophenylalanine
  MOD_RES 4: note = 4-(phosphonodifluoromethyl)phenylalanine
QXFFXR

SEQ ID NO: 181 - protein, 6 aa, Homo sapiens
  REGION 1..6: note = Description of sequence: Example targeting moiety
  MOD_RES 1: note = D-asparagine
  MOD_RES 3: note = 4-fluorophenylalanine
  MOD_RES 4: note = 4-(phosphonodifluoromethyl)phenylalanine
NAXFGR

SEQ ID NO: 182 - protein, 6 aa, Homo sapiens
  REGION 1..6: note = Description of sequence: Example targeting moiety
  MOD_RES 1: note = D-asparagine
  MOD_RES 2: note = D-threonine
  MOD_RES 4: note = 4-(phosphonodifluoromethyl)phenylalanine
NTYFAR

SEQ ID NO: 183 - protein, 6 aa, Homo sapiens
  REGION 1..6: note = Description of sequence: Example targeting moiety
  MOD_RES 1: note = D-Etamic acid
  MOD_RES 3: note = Phenylglycine
  MOD_RES 4: note = 4-(phosphonodifluoromethyl)phenylalanine
EAXFVR

SEQ ID NO: 184 - protein, 6 aa, Homo sapiens
  REGION 1..6: note = Description of sequence: Example targeting moiety
  MOD_RES 2: note = D-valine
  MOD_RES 3: note = Phenylglycine
  MOD_RES 4: note = 4-(phosphonodifluoromethyl)phenylalanine
IVXFAR

SEQ ID NO: 185 - protein, 6 aa, Homo sapiens
  REGION 1..6: note = Description of sequence: Example targeting moiety
  MOD_RES 2: note = D-threonine
  MOD_RES 3: note = Phenylglycine
  MOD_RES 4: note = 4-(phosphonodifluoromethyl)phenylalanine
YTXFAR

SEQ ID NO: 186 - protein, 6 aa, Homo sapiens
  REGION 1..6: note = Description of sequence: Example targeting moiety
  MOD_RES 1: note = D-asparagine
  MOD_RES 2: note = Homoproline
  MOD_RES 3: note = Phenylglycine
  MOD_RES 4: note = 4-(phosphonodifluoromethyl)phenylalanine
NXXFIR

SEQ ID NO: 187 - protein, 6 aa, Homo sapiens
  REGION 1..6: note = Description of sequence: Example targeting moiety
  MOD_RES 1: note = Homoproline
  MOD_RES 2: note = D-asparagine
  MOD_RES 4: note = 4-(phosphonodifluoromethyl)phenylalanine
XNWFHR

SEQ ID NO: 188 - protein, 6 aa, Homo sapiens
  REGION 1..6: note = Description of sequence: Example targeting moiety
  MOD_RES 2: note = Homoproline
  MOD_RES 3: note = D-valine
  MOD_RES 4: note = 4-(phosphonodifluoromethyl)phenylalanine
YXVFIR

SEQ ID NO: 189 - protein, 6 aa, Homo sapiens
  REGION 1..6: note = Description of sequence: Example targeting moiety
  MOD_RES 1: note = D-threonine
  MOD_RES 2: note = D-asparagine
  MOD_RES 4: note = 4-(phosphonodifluoromethyl)phenylalanine
NSAFGR

SEQ ID NO: 190 - protein, 6 aa, Homo sapiens
  REGION 1..6: note = Description of sequence: Example targeting moiety
  MOD_RES 1: note = D-threonine
  MOD_RES 2: note = D-asparagine
  MOD_RES 3: note = D-valine
  MOD_RES 4: note = 4-(phosphonodifluoromethyl)phenylalanine
  MOD_RES 5: note = D-alanine
TNVFAR

SEQ ID NO: 191 - protein, 6 aa, Homo sapiens
  REGION 1..6: note = Description of sequence: Example targeting moiety
  MOD_RES 1: note = D-asparagine
  MOD_RES 2: note = D-threonine
  MOD_RES 3: note = D-valine
  MOD_RES 4: note = 4-(phosphonodifluoromethyl)phenylalanine
  MOD_RES 5: note = D-threonine
NTVFTR

SEQ ID NO: 192 - protein, 6 aa, Homo sapiens
  REGION 1..6: note = Description of sequence: Example targeting moiety
  MOD_RES 3: note = D-threonine
  MOD_RES 4: note = 4-(phosphonodifluoromethyl)phenylalanine
SITFYR

SEQ ID NO: 193 - protein, 6 aa, Homo sapiens
  REGION 1..6: note = Description of sequence: Example targeting moiety
  MOD_RES 1: note = D-asparagine
  MOD_RES 2: note = Phenylglycine
  MOD_RES 3: note = D-asparagine
  MOD_RES 4: note = 4-(phosphonodifluoromethyl)phenylalanine
  MOD_RES 5: note = D-leucine
NXNFLR

SEQ ID NO: 194 - protein, 6 aa, Homo sapiens
  REGION 1..6: note = Description of sequence: Example targeting moiety
  MOD_RES 2..3: note = D-asparagine
  MOD_RES 4: note = 4-(phosphonodifluoromethyl)phenylalanine
  MOD_RES 5: note = Nle
YNNFXR

SEQ ID NO: 195 - protein, 6 aa, Homo sapiens
  REGION 1..6: note = Description of sequence: Example targeting moiety
  MOD_RES 1: note = D-asparagine
  MOD_RES 3: note = D-asparagine
  MOD_RES 4: note = 4-(phosphonodifluoromethyl)phenylalanine
NYNFGR

SEQ ID NO: 196 - protein, 6 aa, Homo sapiens
  REGION 1..6: note = Description of sequence: Example targeting moiety
  MOD_RES 3: note = D-asparagine
  MOD_RES 4: note = 4-(phosphonodifluoromethyl)phenylalanine
AWNFAR

SEQ ID NO: 197 - protein, 6 aa, Homo sapiens
  REGION 1..6: note = Description of sequence: Example targeting moiety
  MOD_RES 1: note = D-valine
  MOD_RES 2: note = D-threonine
  MOD_RES 4: note = 4-(phosphonodifluoromethyl)phenylalanine
VTHFYR

SEQ ID NO: 198 - protein, 6 aa, Homo sapiens
  REGION 1..6: note = Description of sequence: Example targeting moiety
  MOD_RES 2: note = Phenylglycine
  MOD_RES 4: note = 4-(phosphonodifluoromethyl)phenylalanine
  MOD_RES 5: note = Homoproline
PXHFXR

SEQ ID NO: 199 - protein, 6 aa, Homo sapiens
  REGION 1..6: note = Description of sequence: Example targeting moiety
  MOD_RES 1: note = D-asparagine
  MOD_RES 2: note = Phenylglycine
  MOD_RES 4: note = 4-(phosphonodifluoromethyl)phenylalanine
NXHFGR

SEQ ID NO: 200 - protein, 6 aa, Homo sapiens
  REGION 1..6: note = Description of sequence: Example targeting moiety
  MOD_RES 4: note = 4-(phosphonodifluoromethyl)phenylalanine
PAHFGR

SEQ ID NO: 201 - protein, 6 aa, Homo sapiens
  REGION 1..6: note = Description of sequence: Example targeting moiety
  MOD_RES 4: note = 4-(phosphonodifluoromethyl)phenylalanine
AYHFIR

SEQ ID NO: 202 - protein, 6 aa, Homo sapiens
  REGION 1..6: note = Description of sequence: Example targeting moiety
  MOD_RES 1: note = D-asparagine
  MOD_RES 2: note = Homoproline
  MOD_RES 3: note = D-Etamic acid
  MOD_RES 4: note = 4-(phosphonodifluoromethyl)phenylalanine
NXEFYR

SEQ ID NO: 203 - protein, 6 aa, Homo sapiens
  REGION 1..6: note = Description of sequence: Example targeting moiety
  MOD_RES 1: note = D-valine
  MOD_RES 4: note = 4-(phosphonodifluoromethyl)phenylalanine
VSSFTR

SEQ ID NO: 204 - protein, 8 aa, Homo sapiens
  REGION 1..8: note = Description of sequence: Example targeting moiety
  MOD_RES 1: note = D-alanine
  MOD_RES 2: note = MeGly
  MOD_RES 3: note = phosphorylated D-threonine
  MOD_RES 5: note = pipecolic acid
  MOD_RES 6: note = Naphthylalanine
AXTXXYQK

SEQ ID NO: 205 - protein, 9 aa, Homo sapiens
  REGION 1..9: note = Description of sequence: Example targeting moiety
  MOD_RES 1: note = trimesic acid
  MOD_RES 2: note = D-alanine
  MOD_RES 3: note = MeGly
  MOD_RES 4: note = phosphorylated D-threonine
  MOD_RES 5: note = pipecolic acid
  MOD_RES 6: note = Naphthylalanine
  MOD_RES 9: note = 2,3-diaminopropionic acid
XAXTXXRAX

SEQ ID NO: 206 - protein, 10 aa, Homo sapiens
  REGION 1..10: note = Description of sequence: Example targeting moiety
  MOD_RES 1: note = trimesic acid
  MOD_RES 2: note = D-alanine
  MOD_RES 3: note = MeGly
  MOD_RES 4: note = phosphorylated D-threonine
  MOD_RES 5: note = pipecolic acid
  MOD_RES 6: note = Naphthylalanine
  MOD_RES 9: note = D-alanine
  MOD_RES 10: note = 2,3-diaminopropionic acid
XAXTXXRAAX

SEQ ID NO: 207 - protein, 10 aa, Homo sapiens
  REGION 1..10: note = Description of sequence: Example targeting moiety
  MOD_RES 1: note = trimesic acid
  MOD_RES 2: note = D-alanine
  MOD_RES 3: note = MeGly
  MOD_RES 4: note = D-threonine
  MOD_RES 5: note = pipecolic acid
  MOD_RES 6: note = Naphthylalanine
  MOD_RES 9: note = D-alanine
  MOD_RES 9: note = 2,3-diaminopropionic acid
XAXTXXRAAX

SEQ ID NO: 208 - protein, 10 aa, Homo sapiens
  REGION 1..10: note = Description of sequence: Example targeting moiety
  MOD_RES 1: note = trimesic acid
  MOD_RES 2: note = D-alanine
  MOD_RES 3: note = MeGly
  MOD_RES 4: note = D-threonine
  MOD_RES 4: note = D-alanine
  MOD_RES 6: note = Naphthylalanine
  MOD_RES 9: note = D-alanine
  MOD_RES 9: note = 2,3-diaminopropionic acid
XAXTAXRAAX

(19) United States
(12) Patent Application Publication
(10) Pub. No.: US 2026/0265343 A1
(43) Pub. Date: Sep. 10, 2026

(54) MEMBRANE TRANSLOCATION DOMAINS AND USES THEREOF

(71) Applicant: Ohio State Innovation Foundation, Columbus, OH (US)

(72) Inventors: Dehua PEI, Columbus, OH (US); Prabhat BHAT, Columbus, OH (US)

(21) Appl. No.: 18/847,105

(22) PCT Filed: Mar. 17, 2023
(86) PCT No.: PCT/US2023/064659

§ 371 Date: Sep. 13, 2024

Related U.S. Application Data
(60) Provisional application No. 63/320,978, filed on Mar. 17, 2022.

(51) Int. Cl. C07K 14/78; A01N 63/50; A01P 21/00; A61K 47/64; C07K 5/08; C07K 5/10; C07K 7/06

(52) U.S. Cl. CPC C07K 14/78; A01N 63/50; A01P 21/00; A61K 47/6435; C07K 5/08; C07K 5/10; C07K 7/06; C07K 2319/00

(57) ABSTRACT

Described are peptides comprising a membrane translocation domain having one or more cell penetrating peptide motifs, where at least one cell penetrating peptide motif is from 3 to 10 amino acid residues in length and has at least three arginine and/or lysine residues; or where at least one cell penetrating peptide motif is from 3 to 10 amino acid residues in length and has at least two arginine and/or lysine residues and at least one other cell penetrating peptide motif is from 2 to 8 amino acid residues in length and has at least two hydrophobic residues. Compositions comprising a cargo motif bound to the disclosed peptides are also disclosed. Also disclosed are methods of delivering a cargo moiety into a cell comprising contacting the cell with the peptide as disclosed herein.

### CROSS REFERENCE TO RELATED APPLICATIONS

This application claims the benefit of priority to U.S. Provisional Application 63/320,978, filed Mar. 17, 2022, which is incorporated by reference herein in its entirety.

### STATEMENT OF GOVERNMENT SUPPORT

This invention was made with government support under Grant No. GM122459 awarded by the National Institutes of Health. The government has certain rights in the invention.

### REFERENCE TO SEQUENCE LISTING SUBMITTED ELECTRONICALLY

This application contains a sequence listing, which is submitted electronically via EFS-Web as an ASCII formatted sequence listing with a file name “103361-223WO1_ST26.xml” and a creation date of Mar. 17, 2023, and having a size of 392417 bytes. The sequence listing submitted via EFS-Web is part of the specification and is herein incorporated by reference in its entirety.

### BACKGROUND

Effective delivery of biomolecules (e.g., peptides, proteins, and nucleic acids) into the cell interior (e.g., cytosol and nucleus) would open the door to a new generation of therapeutics capable of treating many of the currently intractable diseases. Over the past decades, researchers have explored a wide variety of delivery modalities including cell-penetrating peptides (Muhammad M. N., et al. (2021) Cell penetrating peptides: A versatile vector for co-delivery of drug and genes in cancer. J. Contr. Rel. 330:1220-1228; Heitz, F., et al. (2009) Twenty years of cell-penetrating peptides: from molecular mechanisms to therapeutics. Br. J. Pharmacol. 157:195-206), bacterial toxins (Pavlik, B. J., et al. (2017) Repurposed bacterial toxins for human therapeutics. Curr. Topics Peptide Protein Res. 18:1-15; Shorter S. A., et al. (2017) The potential of toxin-based drug delivery systems for enhanced nucleic acid therapeutic delivery. Expert Op. Drug Deliv. 14(5):685-696), viruses (Wang, D., et al. (2019) Adeno-associated virus vector as a platform for gene therapy delivery. Nat. Rev. Drug Discov. 18:358-378), polyplexes (Ita, K. (2020) Polyplexes for gene and nucleic acid delivery: Progress and bottlenecks, Eur. J Pharm. Sci. 150:105358), liposomes (Pattni. B. S., et al. (2015) New Developments in Liposomal Drug Delivery. Chem. Rev. 115(19):10938-66), and nanoparticles (Mitchell, M. J., et al. (2021) Engineering precision nanoparticles for drug delivery. Nat. Rev. Drug Discov. 20:101-124) for various biomolecular cargos. Although some of the delivery methods have demonstrated clinical successes (e.g., liposomal delivery of mRNA vaccines (Kim, E. M., et al. (2021) Liposomes: Biomedical Applications. Chonnam Med. J 57(1):27-35)), viral delivery of gene therapies (Wang, D., id.), and bacterial toxin-based anticancer agents (Pavlik B. J., id.), significant limitations remain. For example, viral-, liposome-, and nanoparticle-based delivery systems are often sequestrated by the liver and spleen, limiting their biodistribution to other disease tissues, while the nonviral delivery systems additionally face the major challenge of endosomal entrapment (Patra, J. K., et al. (2018) Nano based drug delivery systems: recent developments and future prospects. J. Nanobiotechnol. 16, 71; Pei D., et al. (2019) Overcoming Endosomal Entrapment in Drug Delivery. Bioconjug. Chem. 30(2):273-283).

A family of cyclic CPPs was discovered that enter the mammalian cell by endocytosis and efficiently escape the early endosome into the cytosol by a vesicle budding-and-collapse mechanism (Qian Z., et al. (2013) Efficient delivery of cyclic peptides into mammalian cells with short sequence motifs. ACS Chem. Biol. 8(2):423-31; Qian, Z., et al. (2016) Discovery and Mechanism of Highly Efficient Cyclic Cell-Penetrating Peptides. Biochem. 55(18):2601-2612; Sahni, A., et al. (2020) Cell-Penetrating Peptides Escape the Endosome by Inducing Vesicle Budding and Collapse. ACS Chem. Biol. 15(9):2485-2492). These cyclic CPPs prove to be highly effective for cytosolic delivery of major drug modalities (e.g., small molecules, peptides, proteins, and nucleic acids) in vitro and in vivo. However, the cyclic CPPs contain nonproteinogenic amino acids and must be chemically synthesized and conjugated to a cargo molecule of interest, making them less ideal for protein delivery, as site-specific conjugation of proteins to other entities remains a significant challenge of its own right. To overcome this limitation, short CPP motifs (e.g., Arg-Arg-Arg-Arg-Trp-Trp-Trp or R₄W₃) (SEQ. ID. NO.:133) were genetically inserted into the surface loops of target proteins to render the latter cell-permeable (Chen, K., et al. (2020) Engineering Cell-Permeable Proteins through Insertion of Cell-Penetrating Motifs into Surface Loops. ACS Chem. Biol. 15(9):2568-2576). While this approach avoids the need for posttranslational modification of proteins, it still has a number of drawbacks. First, it generally requires the availability of structural information for a target protein in order to identify the proper surface loop(s) for CPP insertion. Second, even with structural information already available, one may still need to experimentally test several different insertion sites as well as multiple CPPs sequences to find the optimal combination of insertion site and CPP sequence. For some proteins, it may not be possible to generate an insertion mutant without compromising their stability and/or activity. Finally, each target protein is unique in structure and properties and requires a different engineering/optimization campaign. What are needed are highly effective, all-purpose protein delivery systems that can eliminates some or all of the above drawbacks. The compositions and methods disclosed herein address these and other needs.

### SUMMARY

Disclosed herein are compounds, compositions, methods for making and using such compounds and compositions. In one aspect, disclose are peptides comprising a membrane translocation domain having one or more cell penetrating peptide motifs, where at least one cell penetrating peptide motif is from 3 to 10 amino acid residues in length and has at least three arginine and/or lysine residues; or where at least one cell penetrating peptide motif is from 3 to 10 amino acid residues in length and has at least two arginine and/or lysine residues and at least one other cell penetrating peptide motif is from 2 to 8 amino acid residues in length and has at least two hydrophobic residues. Compositions comprising a cargo motif bound to the disclosed peptides are also disclosed. Also disclosed are methods of delivering a cargo moiety into a cell comprising contacting the cell with the peptide as disclosed herein.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects and together with the description serve to explain the principles of the invention.

FIG. 1. Structures of WT FN3 and MTD1-10. The FN3 structure was generated from the PDB file 1ttg and the structures of MTD1-10 were predicted by Phyre2. The inserted CPP motifs of MTD1-10 are highlighted as dark lines.

FIGS. 2A and 2B. Expression and purification of MTD4. FIG. 2A is a FPLC chromatogram showing the elution of MTD4 from a Ni-NTA column (MTD4 elutes as a broad peak); FIG. 2B is a SDS-PAGE showing the expression level and different fractions during purification on a Ni-NTA column. L, molecular-weight markers; U, crude lysate of uninduced cells; I, crude lysate of IPTG-induced cells; CL, crude cell lysate after centrifugation; FT, flow-through fraction; 1-10, Ni-NTA column elution fractions (the intense band is MTD4).

FIGS. 3A-3I. Live-cell confocal microscopic images of HeLa cells after incubation for 2 h with 5 μM FN3^{TMR} (FIG. 3A), MTD2^{TMR} (FIG. 3B), and MTD4^{TMR} (FIG. 3C), MTD4^{TMR} (FIG. 3D), MTD6^{TMR} (FIG. 3E), and MTD7^{TMR} (FIG. 3F), MTD8^{TMR} (FIG. 3G), MTD9^{TMR} (FIG. 3H), or MTD10^{TMR} (FIG. 3I). All images were obtained under the same conditions including the laser power and signal amplification.

FIG. 4. Total cellular uptake efficiency of TMR-labeled protein domains as measured by flow cytometry. The values represent mean fluorescence intensity of the treated cells.

FIG. 5. Western blot analysis of the global pY levels in NIH3T3 cells after treatment with increasing concentrations of MTD4-PTP1B (0-5 μM) for 4 h. Anti-pY antibody 4G10 was used for pY detection. The same membrane was re-blotted with anti-GAPDH antibody to ensure equal loading. L, molecular-weight markers.

FIGS. 6A-6B. Effect of MTD4-RBDV (FIG. 6A), MTD4-NS1 (FIG. 6B), and NS1-MTD4 (FIG. 6C) on the viability of various cancer cell lines cells. Cells were treated with indicated concentrations of fusion protein or vehicle (PBS) for 72 h at 37° C. Cells were then incubated with the Cell Titer Glo solution for 15 min and luminescence was determined. Viability values reported are relative to that of vehicle (PBS)-treated cells.

FIG. 7. Inhibition of Ras-Raf interaction in HEK293T cells by MTD4-RBDV and MTD4-NS1. Cells were transfected with pEF-RLUC8-L15-KrasG12V (or G12D) and pEF-CRAFRBD (1-149)-L15-GFP and incubated for 24 h at 37° C. Cells were then treated with indicated concentrations of fusion protein for 20-24 h. BRET signal was determined after addition of 10 μM coelenterazine 400a as substrate.

FIGS. 8A-8B. Western blot analysis. Effect of MTD4-RBDV (FIG. 8A) and MTD4-NS1 (FIG. 8B) on the phosphorylation of Akt and MEK in MiaPaCa-2 cells. GAPDH was used as a loading control.

FIG. 9. MTD4-RBDV and MTD4-NS1 causes apoptosis of lung cancer H358 cells. Approximately 10,000 cells were treated with indicated concentrations of MTD4-RBDV or MTD4-NS1 for 24 h in presence of 10% FBS and then stained with Alexa Fluor® 488-Annexin V and propidium iodide before flow cytometry analysis.

FIGS. 10A-10D. Live-cell confocal microscopic images of HEK293T cells after incubation for 6 h with buffer (FIG. 10A), 10 μM GFP11 (FIG. 10B), CPP12-GFP11 (FIG. 10C), or MTD4-GFP11 (FIG. 10D).

FIG. 11: Dose-dependent induction of luciferase activity in HEK293T cells transiently expressing LgBit by HiBit or HiBit conjugated to different delivery vehicles. The y axis values are all relative to that of the untreated cells (no HiBit) and represent the mean±SD of three independent experiments.

FIGS. 12A-12C: Live-cell confocal microscopic images of HeLa cells after incubation for 2 h with buffer (FIG. 12A), 5 μM SEP (FIG. 12B), or 5 μM MTD4-SEP (FIG. 12C). Top panel, nuclear stain with Hoechst; bottom panel, fluorescence of SEP.

FIGS. 13A-13B: Dephosphorylation of pY proteins in HEK293T cells by MTD4-PTP1B. (FIG. 13A) Western blot analysis of cell lysates after the cells were treated with buffer, PTP1B (WT; 5 μM), or increasing concentrations of MTD4-PTP1B (0-5 μM) for 6 h. (FIG. 13B) Coomassie blue staining of the SDS-PAGE gel in (FIG. 13A) showing that similar amounts of proteins samples were loaded onto all lanes.

FIG. 14: Biodistribution of MENC protein in different organs of transgenic mice. The green and red channel correspond to signal from EGFP and m-cherry respectively. The fluorescence in different tissues was monitored for mice euthanized after 3 h (EGFP) and 48 h (mCherry).

FIGS. 15A-15B: SDS-PAGE analysis of MTD4 (FIG. 15A) and FN3 (FIG. 15B) after incubation with human serum for varying periods of time (0-24 h). The protein bands in the boxes correspond to MTD4/FN3 and their degradation products.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

Throughout the present specification, the terms “about” and/or “approximately” may be used in conjunction with numerical values and/or ranges. The term “about” is understood to mean those values near to a recited value, as well as the recited value.

Throughout the present specification, numerical ranges are provided for certain quantities. It is to be understood that these ranges comprise all values and subranges therein. Thus, the range “from 50 to 80” includes all possible values therein (e.g., 50, 51, 52, 53, 54, 55, 56, etc.) and all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a polypeptide conjugate” refers to one or more polypeptide conjugates or at least one polypeptide conjugate. As such, the terms “a” (or “an”), “one or more” and “at least one” are used interchangeably herein. In addition, reference to “a polypeptide conjugate” by the indefinite article “a” or “an” does not exclude the possibility that more than one of the polypeptide conjugates is present, unless the context clearly requires that there is one and only one of the polypeptide conjugates.

As used herein, the term “adjacent” refers to two contiguous amino acids, which are connected by a covalent bond. “Adjacent” is also used interchangeably with “consecutive.”

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, “treat,” “treating,” “treatment” and variants thereof, refers to any administration of the polypeptide conjugate of the present disclosure that partially or completely alleviates, ameliorates, prevents, relieves, inhibits, delays onset of, reduces severity of, and/or reduces incidence of one or more symptoms or features of a disease or a condition as described herein.

As used herein, “therapeutically effective” refers to an amount of the polypeptide conjugate or the complex thereof of the present disclosure that can deliver an amount of a therapeutic nucleic acid which confers a therapeutic effect on a patient.

As used herein, “cell penetrating peptide” or “CPP” refers to any peptide which is capable of penetrating a cell membrane. As used herein, “cyclic cell penetrating peptide” or “cCPP” refers to any cyclic peptide which is capable of penetrating a cell membrane.

As used herein, “linker” or “L” refers to a moiety that covalently attaches two or more components of the polypeptide conjugates disclosed herein (e.g., a linker may covalently attach a CPP and a group that binds to a nucleic acid sequence by electrostatic interactions (i.e., P). In some embodiments, the linker can be natural or non-natural amino acid or polypeptide. In other embodiments, the linker is a synthetic compound containing two or more appropriate functional groups suitable to bind, e.g., the CPP and, independently, P. In some embodiments, the linker is about 3 to about 100 (e.g., about 3 to about 20) atoms in linear length (not counting the branched atoms or substituents). In some embodiments, the linker provides about 1 Å to about 400 Å in distance of the two groups to which it connects.

As used herein, “polypeptide” refers to a string of at least two amino acids attached to one another by a peptide bond. There is no upper limit to the number of amino acids that can be included in a polypeptide. Further, polypeptides may include non-natural amino acids, amino acid analogs, or other synthetic molecules that are capable of integrating into a polypeptide.

As used herein, a “monomer” refers to an amino acid residue in a polypeptide. In some embodiments, an amino acid monomer is divalent. In other embodiments, an amino acid monomer may be trivalent if the monomer is further substituted. For example, a cysteine monomer can independently form peptide bonds at the N and C termini, and also form a disulfide bond.

As used herein, an “amino acid-analog” or “analog” (e.g., “arginine-analog”, “lysine-analog” or “histidine-analog”) refers to a variant of an amino acid that retains at least one function of the amino acid, such as the ability to bind an oligonucleotide through electrostatic interactions. Such variants may have an elongated or shorter side chain (e.g., by one or more —CH₂— groups that retains the ability to bind an oligonucleotide through electrostatic interactions, or alternatively, the modification can improve the ability to bind an oligonucleotide through electrostatic interactions. For example, an arginine analog may include an additional methylene or ethylene between the backbone and guanidine/guanidinium group. Other examples include amino acids with one or more additional substituents (e.g., Me, Et, halogen, thiol, methoxy, ethoxy, C1-haloalkyl, C2-haloalkyl, amine, guanidine, etc). The amino acid-analog can be monovalent, divalent, or trivalent.

Throughout the present specification, peptides and amino acid monomers are depicted as charge neutral species. It is to be understood that such species may bear a positive or negative charge depending on the conditions. For example, at pH 7, the N-terminus of an amino acid is protonated and bears a positive charge (—NH₃⁺), and the C-terminus of an amino acid is deprotonated and bears a negative charge (—CO₂⁻). Similarly, the side chains of certain amino acids may bear a positive or negative charge.

Each amino acid can be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Thus, as used herein, the term “amino acid” refers to natural and non-natural amino acids, and analogs and derivatives thereof. Examples of suitable amino acids include, but are not limited to, alanine, allosoleucine, arginine, asparagine, aspartic acid, cysteine, glutamine, glutamic acid, glycine, histidine, isoleucine, leucine, lysine, methionine, naphthylalanine, phenylalanine, proline, pyroglutamic acid, serine, threonine, tryptophan, tyrosine, valine, a derivative, or combinations thereof. Analogs of amino acids encompass that have a structural similar but not identical to an amino acid, e.g., due to a modification to the side chain or backbone on said amino acid. Such modifications may increase the hydrophobicity of the side chain, including elongation of the side chain by one or more hydrocarbons, or increasing the the solvent accessible surface area (SASA as described herein) of an amino acid having an aromatic ring on its side chain, e.g., by conjugating a second aromatic ring or increasing the size of the aromatic ring. Derivatives of amino acids encompass natural and non-natural amino acids that have been modified (e.g., by substitution) to include a hydrophobic group as described herein. For example, a derivative of lysine includes lysine whose side chain has been substituted with alkylcarboxamidyl. These, and others, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| Alanine | Ala (A) | ala (a) |
| Allosoleucine | AIle | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Cyclohexylalanine | Cha | Cha |
| 2,3-diaminopropionic acid | Dap | Dap |
| 4-fluorophenylalanine | Fpa (Σ) | Pfa |
| Glutamic acid | Glu (E) | glu (e) |
| Glutamine | Gln (Q) | gln (q) |
| Glycine | Gly (G) | gly (g) |
| Gistidine | His (H) | his (h) |
| Homoproline (aka pipecolic acid) | Pip (Θ) | pip (θ) |
| Isoleucine | Ile (I) | ile (i) |
| Leucine | Leu (L) | leu (l) |
| Lysine | Lys (K) | lys (k) |
| Methionine | Met (M) | met (m) |
| Naphthylalanine | Nal (Φ) | nal (φ) |
| Norleucine | Nle (Ω) | nle |
| Phenylalanine | Phe (F) | phe (f) |
| Phenylglycine | Phg (Ψ) | phg |
| 4-(phosphonodifluoromethyl)phenylalanine | F₂Pmp (Λ) | f₂pmp |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| 3-(3-benzothienyl)-alanine | Bta | Bta |
| 4-fluorophenylalanine | Fpa or L-Fpa | fpa or D-Fpa |
| 1-naphthylalanine | L-1-Nal or 1-Nal | D-1-Nal or 1-nal |
| 2-naphthylalanine | L-2-Nal or 2-Nal | D-2-Nal or 2-nal |
| 2-pyridylalanine | L-2-Pya or 2-Pya | D-2-Pya or 2-pya |
| 4-pyridylalanine | L-4-Pya or 4-Pya | D-4-Pya or 4-pya |

*single letter abbreviations: when shown in capital letters herein it indicates the L-amino acid form, when shown in lower case herein it indicates the D-amino acid form.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, having from one to forty carbon atoms. Non-limiting examples of C₂-C₄₀ alkylene include ethylene, propylene, n-butylene, pentylene, and the like. Unless stated otherwise specifically in the specification, an alkylene chain can be optionally substituted as described herein.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to forty carbon atoms, and having one or more carbon-carbon double bonds. Non-limiting examples of C₂-C₄₀ alkenylene include ethenylene (—CH═CH—), propenylene, butenylene, and the like. Unless stated otherwise specifically in the specification, an alkenylene chain can be optionally substituted.

“Alkynyl” or “alkynyl group” refers to a straight or branched hydrocarbon chain radical having from two to twelve carbon atoms and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes Cin and C₂ alkynyls. Non-limiting examples of C₂-C₁₂ alkenyl include ethynyl, propynyl, butynyl, pentynyl and the like.

Unless stated otherwise specifically in the specification, an alkyl group can be optionally substituted.

“Alkynylene” or “alkynylene chain” refers to a straight or branched divalent hydrocarbon chain radical, having from two to forty carbon atoms, and having one or more carbon-carbon triple bonds. Non-limiting examples of C₂-C₄₀ alkynylene include ethynylene (—C≡C—), propargylene and the like. Unless stated otherwise specifically in the specification, an alkynylene chain can be optionally substituted.

“Aryl” refers to a hydrocarbon ring system comprising hydrogen, 6 to 40 carbon atoms and at least one aromatic ring. For purposes of this disclosure, the aryl can be a monovalent or a divalent radical (not counting substituents), which can be a monocyclic, bicyclic, tricyclic or tetracyclic ring system, and which can include fused or bridged ring systems. Aryl radicals include, but are not limited to, radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl radical can be divalent when used as a linker or as a part of a linker. Unless stated otherwise specifically in the specification, an aryl group can be optionally substituted.

As used herein “aromatic” refers to an unsaturated cyclic molecule having 4n+2 π electrons, wherein n is any integer. The term “non-aromatic” refers to any unsaturated cyclic molecule which does not fall within the definition of aromatic.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl and rings that are fully unsaturated, partially unsaturated, and fully saturated. In some embodiments, the carbocyclyl can be divalent when used as a linker or as a part of a linker. Unless stated otherwise specifically in the specification, a carbocyclyl group can be optionally substituted.

“Cycloalkyl” refers to a stable non-aromatic monocyclic or polycyclic fully saturated hydrocarbon radical having from 3 to 40 carbon atoms and at least one ring, wherein the ring consists solely of carbon and hydrogen atoms, which can include fused or bridged ring systems. For purposes of this disclosure, the cycloalkyl can be a monovalent or a divalent radical (not counting substituents). Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. In some embodiments, the cycloalkyl radical can be divalent when used as a linker or as a part of a linker. Unless otherwise stated specifically in the specification, a cycloalkyl group can be optionally substituted.

“Cycloalkenyl” refers to a stable non-aromatic monocyclic or polycyclic hydrocarbon radical having from 3 to 40 carbon atoms, at least one ring having, and one or more carbon-carbon double bonds, wherein the ring consists solely of carbon and hydrogen atoms, which can include fused or bridged ring systems. For purposes of this invention, the cycloalkenyl can be a monovalent or a divalent radical (not counting substituents). Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. In some embodiments, the cycloalkenyl radical can be divalent when used as a linker or as a part of a linker. Unless otherwise stated specifically in the specification, a cycloalkenyl group can be optionally substituted.

“Cycloalkynyl” refers to a stable non-aromatic monocyclic or polycyclic hydrocarbon radical having from 3 to 40 carbon atoms, at least one ring, and one or more carbon-carbon triple bonds, wherein the ring consists solely of carbon and hydrogen atoms, which can include fused or bridged ring systems. For purposes of this invention, the cycloalkynyl can be a monovalent or a divalent radical (not counting substituents). Monocyclic cycloalkynyl radicals include, for example, cycloheptynyl, cyclooctynyl, and the like. In some embodiments, the cycloalkynyl radical can be divalent when used as a linker or as a part of a linker. Unless otherwise stated specifically in the specification, a cycloalkynyl group can be optionally substituted.

“Heterocyclyl,” “heterocyclic ring” or “heterocycle” refers to a stable 3- to 20-membered aromatic ring radical which consists of two to twelve carbon atoms and from one to six heteroatoms selected from the group consisting of nitrogen, oxygen and sulfur. For purposes of this invention, the heterocyclyl radical can be a monovalent or a divalent radical (not counting substituents). Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocyclyl radical can be divalent when used as a linker or as a part of a linker. Unless stated otherwise specifically in the specification, a heterocyclyl group can be optionally substituted.

“Heteroaryl” refers to a 5- to 20-membered ring system radical comprising hydrogen atoms, one to fourteen carbon atoms, one to six heteroatoms selected from the group consisting of nitrogen, oxygen and sulfur, and at least one aromatic ring. For purposes of this invention, the heteroaryl radical can be a monovalent or a divalent radical (not counting substituents) and can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). In some embodiments, the heteroaryl radical can be divalent when used as a linker or as a part of a linker. Unless stated otherwise specifically in the specification, a heteroaryl group can be optionally substituted.

The term “ether” used herein refers to a straight or branched divalent radical moiety —[(CH₂)ₘ—O—(CH₂)ₙ]_{z}— wherein each of m, n, and z are independently selected from 1 to 40. Examples include, but are not limited to, polyethylene glycol. Unless stated otherwise specifically in the specification, the ether can be optionally substituted.

The term “substituted” used herein means any of the above groups (i.e., alkylene, alkenylene, alkynylene, aryl, carbocyclyl, cycloalkyl, cycloalkenyl, cycloalkynyl, heterocyclyl, heteroaryl, and/or ether) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORh, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, =NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents. Further, those skilled in the art will recognize that “substituted” also encompasses instances in which one or more atoms on any of the above groups are replaced by a substituent listed in this paragraph, and the substituent forms a covalent bond with the CPP, P, or L. For example, in certain embodiments, any of the above groups can be substituted at a first position with a carboxylic acid (i.e., —C(═O)OH) which forms an amide bond with a lysine in the CPP, or a group can be substituted at a second position with a thiol group which forms a disulfide bond with a cysteine (or amino acid analog having a thiol group).

As used herein, the term “subject” refers to the target of administration, e.g. a subject. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, fish, bird, rodent, or fruit fly. The term does not denote a particular age or sex. Thus, adult and newborn subjects, as well as fetuses, whether male or female, are intended to be covered. In some examples, the subject is a mammal. A patient refers to a subject afflicted with a disease or disorder. The term “patient” includes human and veterinary subjects. In some examples of the disclosed methods, the subject has been diagnosed with a need for treatment of cancer, autoimmune disease, and/or inflammation prior to the administering step. In some examples of the disclosed method, the subject has been diagnosed with cancer prior to the administering step. The term subject also includes a cell, such as an animal, for example human, cell.

As used herein, the term “treatment” refers to the medical management of a patient with the intent to cure, ameliorate, or stabilize a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. In some examples, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the term “prevent” or “preventing” refers to precluding, averting, obviating, forestalling, stopping, or hindering something from happening, especially by advance action.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can treat or prevent cancer. As a further example, “diagnosed with a need for treating or preventing cancer” refers to having been subjected to a physical examination by a person of skill, for example, a physician, and found to have a condition characterized by cancer or other disease wherein treating or preventing cancer would be beneficial to the subject.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to cancer) based upon an earlier diagnosis by a person of skill and thereafter subjected to treatment for the disorder. It is contemplated that the identification can, in some examples, be performed by a person different from the person making the diagnosis. It is also contemplated, in some examples, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In some examples, a preparation can be administered therapeutically; that is, administered to treat an existing disease or condition. In some examples, a preparation can be administered prophylactically; that is, administered for prevention of a disease or condition.

The term “contacting” as used herein refers to bringing a disclosed compound and a target (e.g., a cell, target receptor, transcription factor, or other biological entity) together in such a manner that the compound can affect the activity of the target either directly, i.e., by interacting with the target itself, or indirectly, i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary and can be administered in one or more dose administrations daily, for one or several days. Guidance can be found in the literature for appropriate dosages for given classes of pharmaceutical products. In some examples, a preparation can be administered in a “prophylactically effective amount”; that is, an amount effective for prevention of a disease or condition.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an amino acid residue in a peptide or protein refers to one or more —OC(O)CH(R)NH— units in the peptide or protein.

As used herein, the symbol

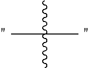

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

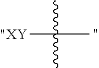

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

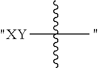

infers that when R³ is “XY”, the point of attachment bond is the same bond as the bond by which R³ is depicted as being bonded to CH₃.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the compounds and compositions disclosed herein include all such possible diastereomers as well as their racemic mixtures, their substantially pure resolved enantiomers, all possible geometric isomers, and pharmaceutically acceptable salts thereof Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds disclosed herein include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds and prodrugs thereof can generally be prepared by carrying out the procedures below, by substituting a readily available isotopically labeled reagent for a non-isotopically labeled reagent.

Disclosed are the components to be used to prepare the compositions disclosed herein as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions disclosed herein. Thus, if there are a variety of additional steps that can be performed it is understood that each of these additional steps can be performed with any specific embodiment or combination of embodiments of the methods disclosed herein.

Reference will now be made in detail to specific aspects of the disclosed materials, compounds, compositions, articles, and methods, examples of which are illustrated in the accompanying Examples and Figures.

### Compositions

Disclosed are cell-permeable peptides and compositions comprising them, which can provide a general vehicle for cytosolic delivery of potentially any peptide or protein cargo as well as other biomolecules. The disclosed compositions can have greater cytosolic delivery efficiency and in vivo stability over simple cell permeable peptides. Further, the disclosed compositions can escape the endosomes of cells more completely, and thus transport conjugated cargo out of the endosome more efficiently than would otherwise occur without the disclosed compositions. The disclosed compositions can also have much broader cargo compatibility (essentially any peptide or protein). And synthesis can be simpler—genetic fusion of the membrane translocation domain to the N-terminus, C-terminus, or an internal position of a cargo protein. Further, the disclosed compositions can have lower likelihood of immunogenicity, and can deliver cargo to just about any eukaryotic (e.g., mammalian and plant) cells, unlike bacterial toxins, which are limited to cells that express the specific receptor(s) for the toxin. The disclosed compositions can also have higher delivery capacity than bacterial toxins, as the delivery capacity of bacterial toxins may be limited by the receptor abundance on target cell surface. In further examples, the disclosed compositions do not require or contain cofactors, such as zinc.

In a specific aspect, disclosed herein are peptides comprising: a membrane translocation domain having one or more cell penetrating peptide motifs, where at least one of the cell penetrating peptide motifs is from 3 to 10 amino acid residues in length and has at least three arginine and/or lysine residues. Unlike methods where a CPP motif is inserted into each target protein, the disclosed compositions and methods involve an engineered membrane translocation domain that can be genetically or synthetically fused to any target cargo of interest. Another strategy disclosed herein involves splitting a CPP motif into two halves and inserting them into two different regions of the membrane translocation domain, resulting in greatly improved cytosolic delivery efficiencies.

### Membrane Translocation Domain

The membrane translocation domain portion of the disclosed peptides can be any membrane translocation domain, a peptide sequence that may traverse a lipid bilayer, that has been modified to contain at least one cell penetrating motifs as described herein. In a preferred example, there are two or three cell penetrating motifs in the membrane translocation domains. For example, at least one cell penetrating peptide motif can be from 3 to 10 amino acid residues in length and have at least three arginine and/or lysine residues, e.g., 4, 5, or 6 arginines and/or lysine residues. Alternatively, at least one cell penetrating peptide motif can be from 3 to 10 amino acid residues in length and have at least two arginine and/or lysine residues and at least one other cell penetrating peptide motif can be from 2 to 8 amino acid residues in length and have at least two hydrophobic residues. When there are two or more cell penetrating peptide motifs, there can be two or more arginine residues and/or lysine residues in a 3 to 10 amino acid span and another cell penetrating peptide motif where there are two or more hydrophobic residues within a 2 to 8 amino acid span. The cell penetrating peptide motifs can be anywhere in the membrane translocation domain.

In some examples, the membrane translocation domain can be a human membrane translocation domain, such as fibronectin type III. In a specific example, the membrane translocation domain has at least 90%, at least 95%, or at least 97% sequence similarity with SEQ. ID. NO.:118. In other examples, the membrane translocation domain is human fibronectin type III having BC, DE, CD, and FG loops and the cell penetrating peptide motif is in one or more of the BC, DE, CD, or FG loops, e.g., the cell penetrating peptide motif is in two of the BC, DE, CD, or FG loops, in particular the BC and FG loops. These loops can be defined as having the following sequences BC=AVTVR (SEQ ID NO.:31); CD=GGNSPVQ (SEQ ID NO.:32); DE=PGSK (SEQ ID NO.:33); FG=GRGDSPAS (SEQ ID NO.:34).

In other examples, the membrane translocation domain can be any stably folded protein, which can preferably be efficiently expressed in bacteria. Some additional examples of membrane translocation domains are the nanobody scaffold, DARPin scaffold, and CTPR protein (the consensus tetratricopeptide repeat; Acc. Chem. Res. 2021, 54, 4166-4177).

### Cell Penetrating Peptide Motif

The cell penetrating peptide (CPP) motif can comprises at least 2 amino acids, at least 3 amino acids, at least 4 amino acids, or at least 6 amino acids, more specifically from 3 to 8, from 3 to 6, from 4 to 8, from 4 to 6, or from 6 to 8 amino acids. In most examples, the CPP motif is substituted into the membrane translocation domain such that the resulting peptide has the same number of amino acids as in native membrane translocation domain.

In some examples, at least two, three, four, five, six, or seven amino acids of the CPP motif are adjacent arginine residues. In a preferred, example there are three, four, or five adjacent arginine residues in a CPP motif. In other examples, the arginie residues are not adjacent in the CPP motif. Each amino acid in the CPP motif can independently be a natural or non-natural amino acid. When such adjacent arginine or lysine residues are the CPP motif, then there need not be any additional CPP motifs, e.g., those with hydrophobic residues, though such a hydrophobic CPP motif can still be used. When the CPP motif contains two argine residues, then it is preferred that there be another CPP motif with at least two hydrophobic residues within 2 to 8 amino acids.

In other examples, at least one, at least, two, at least three, or more amino acids of the CPP motif are hydrophobic amino acids, i.e., have hydrophobic side chains. In some examples, the amino acids having hydrophobic side chains are independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylic acid, cyclohexylalanine, norleucine, 3-(3-benzothienyl)-alanine, 3 -(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, tert-leucine, or nicotinoyl lysine, each of which is optionally substituted with one or more substituents. In particular examples, each amino acid having a hydrophobic side chain is independently an amino acid having an aromatic side chain. In some embodiments, the amino acid having an aromatic side chain is 3-benzothienyl-L-alanine, naphthylalanine, phenylglycine, homophenylalanine, phenylalanine, tryptophan, or tyrosine, each of which is optionally substituted with one or more substituents. Thus, in some examples, the amino acids having hydrophobic side chains are phenylalanine, naphthylalanine, tryptophan, or an analog or derivative thereof naphthylalanine or tryptophan, or analogues or derivatives thereof. In other examples, the CPP motif further comprises at least one phenylalanine, phenylglycine, or histidine, or analogues or derivatives thereof.

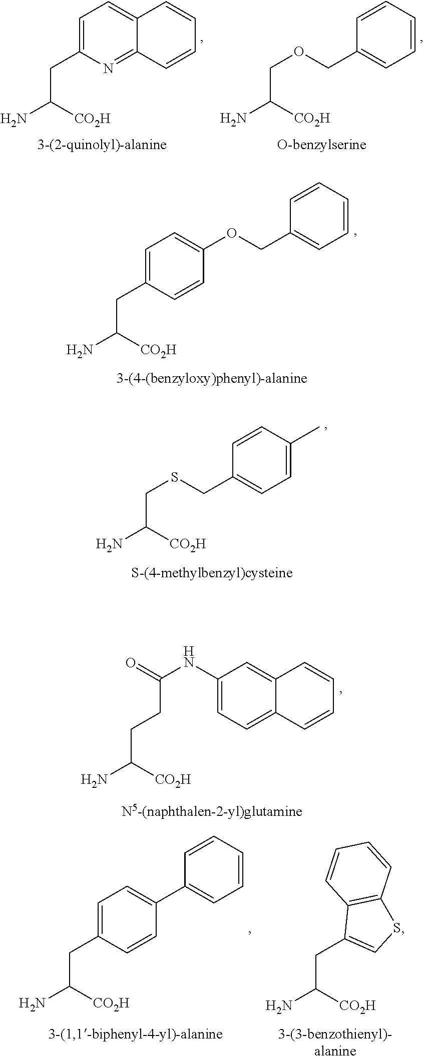

In some examples, the CPP motif can include any combination of at least three adjacent arginines and either at least two amino acids have a hydrophobic side chain selected from an aryl or heteroaryl, wherein the aryl and heteroaryl are optionally substituted, with a total number of amino acids in the CPP motif in the range of from 5 to about 8 amino acids.

In some examples, the membrane translocation domain is human fibronectin type III having BC, DE, CD, and FG loops and the CPP is in one or more of the BC, DE, CD, or FG loops. For example, the CPP motif is in two of the BC, DE, CD, or FG loops. In a specific example, the CPP motif is in the BC and either the DE, CD and FG loops, preferably in the BC and FG loops.

Where there are two or more CPP motifs, one CPP motif can be the 3 to 10 amino acid segment with at least two arginine and/or lysine residues and the other can be a 2 to 8 amino acid segment with at least two hydrophobic residues. For example, the membrane translocation domain can have two or more CPPs and at least one of the motifs is from 2 to 8 amino acid residues and has at least two hydrophobic amino acid residues.

In an example of this, the membrane translocation domain can be human fibronectin type III having BC, DE, CD, and FG loops, and the CPP motifs can be in the BC loop and have from 2 to 8 amino acid residues and has at least two hydrophobic amino acid residues and a CPP motif can be in the FG loop and have from 3 to 10 amino acid residues and has at least three adjacent arginine and/or lysine residues. Alternatively, the CPP motifs can be in the FG loop and have from 2 to 8 amino acid residues and has at least two hydrophobic amino acid residues and a CPP motif can be in the BC loop and have from 3 to 10 amino acid residues and has at least three adjacent arginine and/or lysine residues.

When the CPP motif contains the from 2 to 8 amino acid residues and has at least two hydrophobic amino acid residues, it can be WW, FF, WF, FW, WWW, FFF, WFW, FWF, WWF, WFF, FWW, FFW, WYW, WWH, YWW, or WYH. It is preferable that this CPP motif be in the BC loop. It is further preferable that this CPP motif be WW, FW, WF, WYW, WWW, WWH, YWW, WYH or YWH.

The CPP motif with 3 to 10 amino acid residues and has at least three adjacent arginine and/or lysine residues can contain RRR, RRRR (SEQ ID NO: 159), RRRRR (SEQ ID NO: 160). It can also be any combination of arginine and lysine residues. When this CPP motif is in the FG loop I can be 3-10 residues in length and of any combinations of Arg and Lys (and occasionally other non-acidic residues). The CPP motif (e.g., WWWRRRR) (SEQ ID NO: 161) may be alternatively split, so that some of the Arg/Lys residues are moved from the FG loop into the BC loop (e.g., WWWR . . . RRR (SEQ ID NO: 161), WWWRR . . . RR (SEQ ID NO: 161), WWWRRRR . . . (SEQ ID NO: 161), etc.); The CPP motif (e.g., WWWRRRR) (SEQ ID NO: 161) may be alternatively split, so that some of the hydrophobic residues are moved from the BC loop to the FG loop (e.g., WW . . . WRRR, W . . . WWRRRR, . . . WWWRRRR, etc.) (SEQ ID NO: 161). The CPP motif (e.g., WWWRRRR) can be alternatively split, so that either BC or FG loop contains a combination of hydrophobic and positively charged residues (e.g., WWR . . . WRRR, WWRR . . . WRR, WWRR RRW, RRW . . . WWRR, etc.) (SEQ ID NO: 161).

In specific examples, the CPP motif comprises SEQ ID NOS 104, 105, 11, 112, 113, 114, 115, 116, or 117.

In some examples, the CPP motif can be or comprise any of the sequences listed in Table 2. In some examples, the cell penetrating peptide can be or comprise the reverse of any of the sequences listed in Table 2.

TABLE 2
CPP motif sequences

| | | #R |
|---|---|---|
| SEQ ID NO | CPP sequence | residues |
| SEQ ID NO: 1 | FΦRRR | 3 |
| SEQ ID NO: 2 | FΦRRRC | 3 |
| SEQ ID NO: 3 | FΦRRRU | 3 |
| SEQ ID NO: 4 | RRRΦF | 3 |
| SEQ ID NO: 5 | RRRRΦF | 4 |
| SEQ ID NO: 6 | FΦRRRR | 4 |
| SEQ ID NO: 7 | FφrRrR | 3 |
| SEQ ID NO: 8 | FφrRrR | 3 |
| SEQ ID NO: 9 | FΦRRRR | 4 |
| SEQ ID NO: 10 | fΦRrRr | 4 |
| SEQ ID NO: 11 | RRFRΦR | 4 |
| SEQ ID NO: 12 | FRRRRΦ | 4 |
| SEQ ID NO: 13 | rRFRΦR | 4 |
| SEQ ID NO: 14 | RRΦFRR | 4 |
| SEQ ID NO: 15 | CRRRRFW | 4 |
| SEQ ID NO: 16 | FfQRrRr | 4 |
| SEQ ID NO: 17 | FFΦRRRR | 4 |
| SEQ ID NO: 18 | RFRFRΦR | 4 |
| SEQ ID NO: 19 | URRRRFW | 4 |
| SEQ ID NO: 20 | CRRRRFW | 4 |
| SEQ ID NO: 21 | FΦRRRRQK | 4 |
| SEQ ID NO: 22 | FΦRRRRQC | 4 |
| SEQ ID NO: 23 | fΦRrRrR | 5 |
| SEQ ID NO: 24 | FΦRRRRR | 5 |
| SEQ ID NO: 25 | RRRRΦFDΩC | 4 |
| SEQ ID NO: 26 | FΦRRR | 3 |
| SEQ ID NO: 27 | FWRRR | 3 |
| SEQ ID NO: 28 | RRRΦF | 3 |
| SEQ ID NO: 29 | RRRWF | 3 |
| SEQ ID NO: 30 | FΦRRRR | 4 |
| SEQ ID NO: 31 | FFRRR | 3 |
| SEQ ID NO: 32 | FFrRr | 3 |
| SEQ ID NO: 33 | FFRrR | 3 |
| SEQ ID NO: 34 | FRFRR | 3 |
| SEQ ID NO: 35 | FRRFR | 3 |
| SEQ ID NO: 36 | FRRRF | 3 |
| SEQ ID NO: 37 | GΦRRR | 3 |
| SEQ ID NO: 38 | FFFRA | 1 |
| SEQ ID NO: 39 | FFFRR | 2 |
| SEQ ID NO: 40 | FFRRRR | 4 |
| SEQ ID NO: 41 | FRRFRR | 4 |
| SEQ ID NO: 42 | FRRRFR | 5 |
| SEQ ID NO: 43 | RFFRRR | 4 |
| SEQ ID NO: 44 | RFRRFR | 4 |
| SEQ ID NO: 45 | FRFRRR | 4 |
| SEQ ID NO: 46 | FFFRRR | 3 |
| SEQ ID NO: 47 | FFRRRF | 3 |
| SEQ ID NO: 48 | FRFFRR | 3 |
| SEQ ID NO: 49 | RRFFFR | 3 |
| SEQ ID NO: 50 | FFRFRR | 3 |
| SEQ ID NO: 51 | FFRRFR | 3 |
| SEQ ID NO: 52 | FRRFFR | 3 |
| SEQ ID NO: 53 | FRRFRF | 3 |
| SEQ ID NO: 54 | FRFRFR | 3 |
| SEQ ID NO: 55 | RFFRFR | 3 |
| SEQ ID NO: 56 | GΦRRRR | 4 |
| SEQ ID NO: 57 | FFFRRRR | 4 |
| SEQ ID NO: 58 | RFFRRRR | 5 |
| SEQ ID NO: 59 | RRFFRRR | 5 |
| SEQ ID NO: 60 | RFFFRRR | 4 |
| SEQ ID NO: 61 | RRFFFRR | 4 |
| SEQ ID NO: 62 | FFRRFRR | 4 |
| SEQ ID NO: 63 | FFRRRRF | 4 |
| SEQ ID NO: 64 | FRRFFRR | 4 |
| SEQ ID NO: 65 | FFFRRRRR | 5 |
| SEQ ID NO: 66 | FFFRRRRRR | 6 |
| SEQ ID NO: 67 | FΦRrRr | 4 |
| SEQ ID NO: 68 | XXRRRR | 4 |
| SEQ ID NO: 69 | FfFRrR | 3 |
| SEQ ID NO: 70 | fFfrRr | 2 |
| SEQ ID NO: 71 | fFfRrR | 3 |
| SEQ ID NO: 72 | FfFrRr | 3 |
| SEQ ID NO: 73 | fFφrRr | 3 |
| SEQ ID NO: 74 | fΦfrRr | 3 |
| SEQ ID NO: 75 | φFfrRr | 3 |
| SEQ ID NO: 76 | FΦrRr | 3 |
| SEQ ID NO: 77 | fΦrRr | 3 |
| SEQ ID NO: 78 | Ac-Lys-fFRrRrD | 4 |
| SEQ ID NO: 79 | Ac-Dap-fFRrRrD | 4 |
| SEQ ID NO: 80 | Pip-Nal-Arg-Glu-arg-arg-glu | 3 |
| SEQ ID NO: 81 | Pip-Nal-Arg-Arg-arg-arg-glu | 3 |
| SEQ ID NO: 82 | Pip-Nal-Nal-Arg-arg-arg-glu | 3 |
| SEQ ID NO: 83 | Pip-Nal-Nal-Arg-arg-arg-Glu | 3 |
| SEQ ID NO: 84 | Pip-Nal-Phe-Arg-arg-arg-glu | 3 |
| SEQ ID NO: 85 | Pip-Nal-Phe-Arg-arg-arg-Glu | 3 |
| SEQ ID NO: 86 | Pip-Nal-phe-Arg-arg-arg-glu | 3 |
| SEQ ID NO: 87 | Pip-Nal-phe-Arg-arg-arg-Glu | 3 |
| SEQ ID NO: 88 | Pip-Nal-nal-Arg-arg-arg-Glu | 3 |
| SEQ ID NO: 89 | Pip-Nal-nal-Arg-arg-arg-glu | 3 |
| SEQ ID NO: 90 | [P-Cha-r-Cha-r-Cha-r-Cha-r-G]^{d} | 4 |
| SEQ ID NO: 91 | Tm(SvP-F2Pmp-H)-Dap-(FΦRRRR- | 4 |
| | Dap)]^{f} | |
| SEQ ID NO: 92 | [Tm(a-Sar-D-pThr-Pip-ΦRAa)-Dap- | 5 |
| | (FΦRRRR-Dap)]^{f} | |
| SEQ ID NO: 93 | [CRRSRRGCGRRSRRCG]^{g} | 8 |
| SEQ ID NO: 94 | [K(Dod)RRRR] | 4 |
| SEQ ID NO: 95 | LKKLCKLLKKLCKLAG | 0 |
| SEQ ID NO: 96 | RRRR-[KRRRE]^{c} | 7 |
| SEQ ID NO: 97 | RRR-[KRRRRE]^{c} | 7 |
| SEQ ID NO: 98 | RR-[KRRRRRE]^{c} | 7 |
| SEQ ID NO: 99 | R-[KRRRRRRE]^{c} | 7 |
| SEQ ID NO: 100 | [CRCRCRCR] | 4 |
| SEQ ID NO: 101 | [Pra-LRKRLRKFRN-AzK]^{h} | |
| SEQ ID NO: 102 | T-Dap-[Dap-Dap-f-L-Dap-Dap-T] | |
| SEQ ID NO: 103 | T-Agp-[Dap-Agp-f-L-Agp-Agp-T] | |

Φ = L-naphthylalanine; φ = D-naphthylalanine; Ω = L-norleucine; r = D-arginine; F = L-phenylalanine; f = D-phenylalanine; q = D-glutamine; X = L-4-fluorophenylalanine; Dap = L-2,3-diaminopropionic acid; Sar, sarcosine; F2Pmp, L-difluorophosphonomethyl phenylalanine; Dod, dodecanoyl; Pra, L-propargylglycine; AzK, L-6-Azido-2-amino-hexanoic; Agp, L-2-amino-3-guanidinylpropionic acid; ^{b}Cyclization between Pim and Nlys; ^{c}Cyclization between Lys and Glu; ^{d}Macrocyclization by multicomponent reaction with aziridine aldehyde and isocyanide; ^{e}Cyclization between the main-chain of Gln residue; ^{f}N-terminal amine and side chains of two Dap residues bicyclized with Tm; ^{g}Three Cys side chains bicyclized with tris(bromomethyl)benzene; ^{h}Cyclization by the click reaction between Pra and Azk.

The chirality of the amino acids can be selected to improve cytosolic uptake efficiency. In some embodiments, at least two of the amino acids have the opposite chirality. In some embodiments, the at least two amino acids having the opposite chirality can be adjacent to each other. In some embodiments, at least three amino acids have alternating stereochemistry relative to each other. In some embodiments, the at least three amino acids having the alternating chirality relative to each other can be adjacent to each other. In some embodiments, at least two of the amino acids have the same chirality. In some embodiments, the at least two amino acids having the same chirality can be adjacent to each other. In some embodiments, at least two amino acids have the same chirality and at least two amino acids have the opposite chirality. In some embodiments, the at least two amino acids having the opposite chirality can be adjacent to the at least two amino acids having the same chirality. Accordingly, in some embodiments, adjacent amino acids in the cCPP can have any of the following sequences: D-L; L-D; D-L-L-D (SEQ ID NO:153); L-D-D-L (SEQ ID NO: 154); L-D-L-L-D (SEQ ID NO: 155); D-L-D-D-L (SEQ ID NO: 156); D-L-L-D-L (SEQ ID NO:157); or L-D-D-L-D (SEQ ID NO:158).

### Cargo Moiety

Also described are peptides as disclosed herein, but that further comprise a cargo moiety linked to the membrane translocation domain. The cargo moiety can be linked to an amino group (e.g., N-terminus), a carboxylate group (e.g., C-terminus), or a side chain of one or more amino acids in the in the membrane translocation domain.

When the cargo moiety is attached to the side chain of an amino acid in the membrane translocation domain, the membrane translocation domain includes an amino acid having a side chain with a suitable functional group to form a covalent bond (conjugation) with the cargo, or a side chain which may be modified to provide a suitable functional group (e.g., via conjugation of a linker) that forms a covalent bond with the cargo. In some embodiments, the amino acid on membrane translocation domain which has a side chain suitable conjugation of the cargo is a cysteine residue, glutamic acid residue, an aspartic acid residue, a lysine residue, or a 2,3-diaminopropionic acid residue. In such embodiments, the cargo may be directly conjugated to the side chain of the amino acid (e.g., by forming a disulfide bond with a cysteine residue or an amide bond with a glutamic acid residue or a 2,3-diaminopropionic acid residue) or the cargo may be conjugated to the amino acid side chain through a linker (e.g., PEG).

The cargo moiety can comprise any cargo of interest, for example a linker moiety, a detectable moiety, a therapeutic moiety, a targeting moiety, and the like, or any combination thereof. In some examples, the cargo moiety can comprise one or more additional amino acids (e.g., K, UK, TRV); a linker (e.g., bifunctional linker LC-SMCC); coenzyme A; phosphocoumaryl amino propionic acid (pCAP); 8-amino-3,6-dioxaoctanoic acid (miniPEG); L-2,3-diaminopropionic acid (Dap or J); L-j-naphthylalanine; L-pipecolic acid (Pip); sarcosine; trimesic acid; 7-amino-4-methylcourmarin (Amc); fluorescein isothiocyanate (FITC); L-2-naphthylalanine; norleucine; 2-aminobutyric acid; Rhodamine B (Rho); Dexamethasone (DEX); or combinations thereof.

### Detectable Moiety

The detectable moiety can comprise any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, an isotope detectable spin resonance label, a paramagnetic moiety, a chromophore, or any combination thereof. In some embodiments, the label is detectable without the addition of further reagents.

In some embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene, perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetrapheny metrylbenzoporphyrin; Pd (II) octaethylporphyrin ketone; Pt (II) octaethylporphyrin ketone; Pd (II) meso-tetra(pentafluorophenyl)porphyrin; Pt (II) meso-tetra (pentafluorophenyl) porphyrin; Ru (II) tris(4,7-diphenyl-1,10-phenanthroline) (Ru (dpp)₃); Ru (II) tris(1,10-phenanthroline) (Ru(phen)₃), tris(2,2′-bipyridine)rutheniurn (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); indium (III) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitirophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluorescein; eosin; 2′,7′-dichlorofluorescein; 5(6)-carboxy-fluorecsein; carboxynaphthofluorescein; 8-hydroxypyrene-1,3,6-trisulfonic acid; semi-naphthorhodafluor; semi-naphthofluorescein; tris (4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; dioctadecylcycloxacarbocyanine; fluorenylmethyloxycarbonyl chloride; 7-amino-4-methylcourmarin (Amc); green fluorescent protein (GFP); and derivatives or combinations thereof.

In some examples, the detectable moiety can comprise Rhodamine B (Rho), fluorescein isothiocyanate (FITC), 7-amino-4-methylcourmarin (Amc), green fluorescent protein (GFP), naphthofluorescein (NF), or derivatives or combinations thereof.

The detectible moiety can be attached to the cell penetrating peptide moiety at the amino group, the carboxylate group, or the side chain of any of the amino acids of the cell penetrating peptide moiety (e.g., at the amino group, the carboxylate group, or the side chain of any amino acid in the CPP).

### Therapeutic Moiety

The disclosed compounds can also comprise a therapeutic moiety. In some examples, the cargo moiety comprises a therapeutic moiety. The detectable moiety can be linked to a therapeutic moiety or the detectable moiety can also serve as the therapeutic moiety. Therapeutic moiety refers to a group that when administered to a subject will reduce one or more symptoms of a disease or disorder.

The therapeutic moiety can comprise a wide variety of drugs, including antagonists, for example enzyme inhibitors, and agonists, for example a transcription factor which results in an increase in the expression of a desirable gene product (although as will be appreciated by those in the art, antagonistic transcription factors can also be used), are all included. In addition, therapeutic moiety includes those agents capable of direct toxicity and/or capable of inducing toxicity towards healthy and/or unhealthy cells in the body.

Also, the therapeutic moiety can be capable of inducing and/or priming the immune system against potential pathogens.

The therapeutic moiety can, for example, comprise an anticancer agent, antiviral agent, antimicrobial agent, anti-inflammatory agent, immunosuppressive agent, anesthetics, or any combination thereof.

In some examples, the therapeutic moiety can be a tumor suppressor, small molecule or peptide based inhibitor, an enzyme for intracellular enzyme replacement therapy, an oligonucleotide.

The therapeutic moiety can comprise an anticancer agent. Example anticancer agents include 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot. Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred. Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The therapeutic moiety can also comprise a biopharmaceutical such as, for example, an antibody.

In some examples, the therapeutic moiety can comprise an antiviral agent, such as ganciclovir, azidothymidine (AZT), lamivudine (3TC), etc.

In some examples, the therapeutic moiety can comprise an antibacterial agent, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisonicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

In some examples, the therapeutic moiety can comprise an anti-inflammatory agent.

In some examples, the therapeutic moiety can comprise dexamethasone (Dex).

In other examples, the therapeutic moiety comprises a therapeutic protein. The protein may be genetically fused to the N- or C-terminus of the MTD. A synthetic peptide containing unnatural amino acids can also be chemically conjugated to a side chain, e.g. a unique cysteine at the C-terminus of MTD. It is disclosed herein to deliver such enzymes/proteins to human cells by linking to the enzyme/protein to one of the disclosed cell penetrating peptide motifs or the MTD. The disclosed cell penetrating peptide motifs have been tested with proteins (e.g., GFP, PTP1B, actin, calmodulin, troponin C) and shown to work.

### Targeting Moiety

In some examples, the therapeutic moiety comprises a targeting moiety. The targeting moiety can comprise, for example, a sequence of amino acids that can target one or more enzyme domains. In some examples, the targeting moiety can comprise an inhibitor against an enzyme that can play a role in a disease, such as cancer, cystic fibrosis, diabetes, obesity, or combinations thereof. For example, the targeting moiety can comprise any of the sequences listed in Table 3.

TABLE 3
Example targeting moieties

| | | SEQ ID |
|---|---|---|
| Abbreviation * | Sequence | NO |
| PΘGΛYR | Pro-Pip-Gly-F₂Pmp-Tyr-Arg | 162 |
| SΘIΛΛR | Ser-Pip-Ile-F₂Pmp-F₂Pmp-Arg | 163 |
| IHIΛIR | Ile-His-Ile-F₂Pmp-Ile-Arg | 164 |
| AalΛΘR | Ala-(D-Ala)-Ile-F₂Pmp-Pip-Arg | 165 |
| ΣSΘΛvR | Fpa-Ser-Pip-F₂Pmp-(D-Val)-Arg | 166 |
| ΘnPΛAR | Pip-(D-Asn)-Pro-F₂Pmp-Ala-Arg | 167 |
| TΨAΛGR | Tyr-Phg-Ala-F₂Pmp-Gly-Arg | 168 |
| AHIΛaR | Ala-His-Ile-F₂Pmp-(D-Ala)-Arg | 169 |
| GnGΛpR | Gly-(D-Asn)-Gly-F₂Pmp-(D-Pro)-Arg | 170 |
| fQΘΛAR | (D-Phe)-Gln-Pip-F₂Pmp-Ile-Arg | 171 |
| SPGΛHR | Ser-Pro-Gly-F₂Pmp-His-Arg | 172 |
| ΘΥIΛHR | Pip-Tyr-Ile-F₂Pmp-His-Arg | 173 |
| SvPΛHR | Ser-(D-Val)-Pro-F₂Pmp-His-Arg | 174 |
| AIPΛnR | Ala-Ile-Pro-F₂Pmp-(D-Asn)-Arg | 175 |
| ΣSIΛQF | Fpa-Ser-Ile-F₂Pmp-Gln-Arg | 176 |
| AaΨΛfR | Ala-(D-Ala)-Phg-F₂Pmp-(D-Phe)-Arg | 177 |
| ntΨΛΨR | (D-Asn)-(D-Thr)-Phg-F₂Pmp-Phg-Arg | 178 |
| IPΨΛΩN | Ile-Pro-Phg-F₂Pmp-Nle-Arg | 179 |
| QΘΣΛΘR | Gln-Pip-Fpa-F₂Pmp-Pip-Arg | 180 |
| nAΣΛGR | (D-Asn)-Ala-Fpa-F₂Pmp-Gly-Arg | 181 |
| ntYΛAR | (D-Asn)-(D-Thr)-Tyr-F₂Pmp-Ala-Arg | 182 |
| eAΨΛvR | (D-Glu)-Ala-Phg-F₂Pmp-(D-Val)-Arg | 183 |
| IvΨΛAR | Ile-(D-Val)-Phg-F₂Pmp-Ala-Arg | 184 |
| ΥtΨΛAR | Tyr-(D-Thr)-Phg-F₂Pmp-Ala-Arg | 185 |
| nΘΨΛIR | (D-Asn)-Pip-Phg-F₂Pmp-Ile-Arg | 186 |
| ΘnWΛHR | Pip-(D-Asn)-Trp-F₂Pmp-His-Arg | 187 |
| YΘvΛIR | Tyr-Pip-(D-Val)-F₂Pmp-Ile-Arg | 188 |
| nSAΛGR | (D-Asn)-Ser-(D-Ala)-F₂Pmp-Gly-Arg | 189 |
| tnvΛaR | (D-Thr)-(D-Asn)-(D-Val)-F₂Pmp-(D-Ala)-Arg | 190 |
| ntvΛtR | (D-Asn)-(D-Thr)-(D-Val)-F₂Pmp-(D-Thr)-Arg | 191 |
| SItΛYR | Ser-Ile-(D-Thr)-F₂Pmp-Tyr-Arg | 192 |
| nΣnΛIR | (D-Asn)-Fpa-(D-Asn)-F₂Pmp-(D-Leu)-Arg | 193 |
| ΥnnΛΩR | Tyr-(D-Asn)-(D-Asn)-F₂Pmp-Nle-Arg | 194 |
| nYnΛGR | (D-Asn)-Tyr-(D-Asn)-F₂Pmp-Gly-Arg | 195 |
| AWnAAR | Ala-Trp-(D-Asn)-F₂Pmp-Ala-Arg | 196 |
| vtHΛYR | (D-Val)-(D-Thr)-His-F₂Pmp-Tyr-Arg | 197 |
| PYHΛΘR | Pro-Phg-His-F₂Pmp-Pip-Arg | 198 |
| nYHΛGR | (D-Asn)-Phg-His-F₂Pmp-Gly-Arg | 199 |
| PAHΛGR | Pro-Ala-His-F₂Pmp-Gly-Arg | 200 |
| AYHΛIR | Ala-Tyr-His-F₂Pmp-Ile-Arg | 201 |
| nΘeΛYR | (D-Asn)-Pip-(D-Glu)-F₂Pmp-Tyr-Arg | 202 |
| vSSΛtR | (D-Val)-Ser-Ser-F₂Pmp-(D-Thr)-Arg | 203 |
| aΞt′ϑΦ′YNK | ((D-Ala)-Sar-(D-pThr)-Pp-Nal-Tyr-Gln)-Lys | 204 |
| Tm(aΞt′ϑΦ′RA)Dap | Tm((D-Ala)-Sar-(D-pThr)-Pp-Nal-Arg-Ala)- | 205 |
| | Dap | |
| Tm(aΞt′ϑΦ′RAa)Dap | Tm((D-Ala)-Sar-(D-pThr)-Pp-Nal-Arg-Ala-(D- | 206 |
| | Ala))-Dap | |
| Tm(aΞtϑΦ′RAa)Dap | Tm((D-Ala)-Sar-(D-Thr)-Pp-Nal-Arg-Ala-(D- | 207 |
| | Ala))-Dap | |
| Tm(aΞtaΦ′RAa)Dap | Tm((D-Ala)-Sar-(D-Thr)-(D-Ala)-Nal-Arg-Ala- | 208 |
| | (D-Ala))-Dap | |

*Fpa, Σ: L-4-fluorophenylalanine; Pip, Θ: L-homoproline; Nle, Ω: L-norleucine; Phg, Ψ L-phenylglycine; F₂Pmp, Λ: L-4-(phosphonodifluoromethyl)phenylalanine; Dap, L-2,3-diaminopropionic acid; Nal, Φ′: L-β-naphthylalanine; Pp, ϑ: L-pipecolic acid; Sar, Ξ: sarcosine; Tm, trimesic acid.

The targeting moiety and cell penetrating peptide moiety can overlap. That is, the residues that form the cell penetrating peptide moiety can also be part of the sequence that forms the targeting moiety, and vice a versa.

The therapeutic moiety can be attached to the cell penetrating peptide moiety at the amino group, the carboxylate group, or the side chain of any of the amino acids of the cell penetrating peptide moiety (e.g., at the amino group, the carboxylate group, or the side chain or any of amino acid of the CPP). In some examples, the therapeutic moiety can be attached to the detectable moiety.

In some examples, the therapeutic moiety can comprise a targeting moiety that can act as an inhibitor against Ras (e.g., K-Ras), PTP1B, Pin1, Grb2 SH2, CAL PDZ, and the like, or combinations thereof.

Ras is a protein that in humans is encoded by the RAS gene. The normal Ras protein performs an essential function in normal tissue signaling, and the mutation of a Ras gene is implicated in the development of many cancers. Ras can act as a molecular on/off switch, once it is turned on Ras recruits and activates proteins necessary for the propagation of growth factor and other receptors' signal. Mutated forms of Ras have been implicated in various cancers, including lung cancer, colon cancer, pancreatic cancer, and various leukemias.

Protein-tyrosine phosphatase 1B (PTP1B) is a prototypical member of the PTP superfamily and plays numerous roles during eukaryotic cell signaling. PTP1B is a negative regulator of the insulin signaling pathway, and is considered a promising potential therapeutic target, in particular for the treatment of type II diabetes. PIP1B has also been implicated in the development of breast cancer.

Pin1 is an enzyme that binds to a subset of proteins and plays a role as a post phosphorylation control in regulating protein function. Pin1 activity can regulate the outcome of proline-directed kinase signaling and consequently can regulate cell proliferation and cell survival. Deregulation of Pin1 can play a role in various diseases. The up-regulation of Pin1 may be implicated in certain cancers, and the down-regulation of Pin1 may be implicated in Alzheimer's disease. Inhibitors of Pin1 can have therapeutic implications for cancer and immune disorders.

Grb2 is an adaptor protein involved in signal transduction and cell communication. The Grb2 protein contains one SH2 domain, which can bind tyrosine phosphorylated sequences. Grb2 is widely expressed and is essential for multiple cellular functions. Inhibition of Grb2 function can impair developmental processes and can block transformation and proliferation of various cell types.

It was recently reported that the activity of cystic fibrosis membrane conductance regulator (CFTR), a chloride ion channel protein mutated in cystic fibrosis (CF) patients, is negatively regulated by CFTR-associated ligand (CAL) through its PDZ domain (CAL-PDZ) (Wolde, M et al. J. Biol. Chem. 2007, 282, 8099). Inhibition of the CFTR/CAL-PDZ interaction was shown to improve the activity of ΔPhe508-CFTR, the most common form of CFTR mutation (Cheng, S H et al. Cell 1990, 63, 827; Kerem, B S et al. Science 1989, 245, 1073), by reducing its proteasome-mediated degradation (Cushing, P R et al. Angew. Chem. Int. Ed. 2010, 49, 9907). Thus, disclosed herein is a method for treating a subject having cystic fibrosis by administering an effective amount of a compound or composition disclosed herein. The compound or composition administered to the subject can comprise a therapeutic moiety that can comprise a targeting moiety that can act as an inhibitor against CAL PDZ.

In some embodiments, the therapeutic moiety is a nucleic acid. In some embodiments, the nucleic acid is an antisense compound. In some embodiments, the antisense compound is selected from the group consisting of an antisense oligonucleotide, a small interfering RNA (siRNA), microRNA (miRNA), a ribozyme, an immune stimulating nucleic acid, an antagomir, an antimir, a microRNA mimic, a supermir, a Ul adaptor, and an aptamer.

Also disclosed herein are compositions comprising the compounds described herein.

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Linker

In various embodiments, the linker is covalently bound to an amino acid on the membrane translocation domain. The linker may be any moiety which conjugates two or more of the membrane translocation domain to the cargo moiety. In some embodiments, the linker can be an amino acid. In other embodiments, the precursor to the linker can be any appropriate molecule which is capable of forming two or more bonds with amino acids in the membrane translocation domain and cargo moiety. Thus, in various embodiments, the precursor of the linker has two or more functional groups, each of which are capable of forming a covalent bond to the membrane translocation domain and cargo moiety. For example, the linker can be covalently bound to the N-terminus, C-terminus, or side chain, or combinations thereof, of any amino acid in the membrane translocation domain. In particular embodiments, the linker forms a covalent bond between the membrane translocation domain and cargo moiety.

In some embodiments, the linker is selected from the group consisting of at least one amino acid, alkylene, alkenylene, alkynylene, aryl, cycloalkyl, cycloalkenyl, cycloalkynyl, heterocyclyl, heteroaryl, ether, each of which can be optionally substituted as defined above. For example, each of these likers can be from 1 to 500 atoms in length, e.g., from 1 to 100, from 1 to 250, from 10 to 200, from 25 to 300 atoms in length. Non-limiting examples of linkers include polyethylene glycol, optionally conjugated to a lysine residue. In other examples, the linker can be a bivalent or trivalent C₁-C₅₀ saturated or unsaturated, straight or branched alkyl, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heteroaryl, cycloalkyl, or cycloalkenyl.

In some embodiments, the linker is covalently bound to the N or C-terminus of an amino acid on CPP motif, or to a side chain of glutamine, asparagine, or lysine, or a modified side chain of glutamine or asparagine (e.g., a reduced side chain having an amino group). In particular embodiments, the linker forms a bond with the side chain of glutamine on the CPP motif. In other particular embodiments, the linker described herein has a structure of L-1 or L-2:

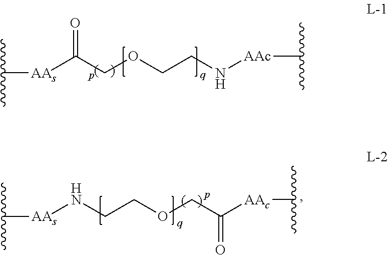

wherein
AAs is a side chain or terminus of an amino acid on the peptide or staple;
AA_{c} is a side chain or terminus of an amino acid of the cCPP;
p is an integer from 0 to 10; and
q is an integer from 1 to 50.

In other embodiments the linker can be a proteolytically stable peptide sequence, such as (GGS)n, (GGGS)n, (GSS)n, or (PAS)n, where n is 0-100.

In some embodiments, the linker is capable of releasing the cargo moiety from the membrane translocation domain after the polypeptide conjugate enters the cytosol of the cell. In some embodiments, the linker contains a group, or forms a group after binding to membrane translocation domain and cargo moiety that is cleaved after cytosolic uptake of the polypeptide conjugate to thereby release the cargo moiety. Non-limiting examples of physiologically cleavable linking group include carbonate, thiocarbonate, thioether, thioester, disulfide, sulfoxide, hydrazine, protease-cleavable dipeptide linker, and the like.

For example, in embodiments, the linker is covalently bound to membrane translocation domain through a disulfide bond e.g., with the side chain of cysteine or cysteine analog located in the membrane translocation domain or cargo moiety. In some embodiments, the disulfide bond is formed between a thiol group on a precursor of the linker, and the side chain of cysteine or an amino acid analog having a thiol group on the peptide, wherein the bond to hydrogen on each of the thiol groups is replaced by a bond to a sulfur atom. Non-limiting examples of amino acid analogs having a thiol group which can be used with the polypeptide conjugates disclosed herein are discussed above.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by expressing and purifying like any other proteins. See Chen, K., & Pei, D. (2020). Engineering Cell-Permeable Proteins through Insertion of Cell-Penetrating Motifs into Surface Loops. ACS chemical biology, 15(9), 2568-2576, which is incorporated by reference herein in its entirety for its teachings of methods of preparing proteins. Other methods for preparing the disclosed compositions involve solid phase peptide synthesis wherein the amino acid α-N-terminal is protected by an acid- or base-sensitive protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobomyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for aspartic acid and glutamic acid, benzyl and t-butyl and for cysteine, triphenylmethyl (trityl). In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP). 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminal in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF.

The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either in successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

### Methods of Use

Also provided herein are methods of use of the compounds or compositions described herein. Also provided herein are methods for treating a disease or pathology in a subject in need thereof comprising administering to the subject an effective amount of any of the compounds or compositions described herein.

Also provided herein are methods of treating, preventing, or ameliorating cancer in a subject. The methods include administering to a subject an effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

The methods of treatment or prevention of cancer described herein can further include treatment with one or more additional agents (e.g., an anti-cancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil. Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The additional anti-cancer agent can also include biopharmaceuticals such as, for example, antibodies.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of killing a tumor cell in a subject. The method includes contacting the tumor cell with an effective amount of a compound or composition as described herein, and optionally includes the step of irradiating the tumor cell with an effective amount of ionizing radiation. Additionally, methods of radiotherapy of tumors are provided herein. The methods include contacting the tumor cell with an effective amount of a compound or composition as described herein, and irradiating the tumor with an effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. An effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection. Prophylactic administration can be used, for example, in the chemopreventative treatment of subjects presenting precancerous lesions, those diagnosed with early stage malignancies, and for subgroups with susceptibilities (e.g., family, racial, and/or occupational) to particular cancers. Therapeutic treatment involves administering to a subject a therapeutically effective amount of the compounds and compositions or pharmaceutically acceptable salts thereof as described herein after cancer is diagnosed.

In some examples of the methods of treating of treating, preventing, or ameliorating cancer or a tumor in a subject, the compound or composition administered to the subject can comprise a therapeutic moiety that can comprise a targeting moiety that can act as an inhibitor against Ras (e.g., K-Ras), PTP1B, Pin1, Grb2 SH2, or combinations thereof.

The disclosed subject matter also concerns methods for treating a subject having a metabolic disorder or condition. In one embodiment, an effective amount of one or more compounds or compositions disclosed herein is administered to a subject having a metabolic disorder and who is in need of treatment thereof. In some examples, the metabolic disorder can comprise type II diabetes. In some examples of the methods of treating of treating, preventing, or ameliorating the metabolic disorder in a subject, the compound or composition administered to the subject can comprise a therapeutic moiety that can comprise a targeting moiety that can act as an inhibitor against PTP1B. In one particular example of this method the subject is obese and the method comprises treating the subject for obesity by administering a composition as disclosed herein.

The disclosed subject matter also concerns methods for treating a subject having cystic fibrosis. In one embodiment, an effective amount of one or more compounds or compositions disclosed herein is administered to a subject having cystic fibrosis and who is in need of treatment thereof. In some examples of the methods of treating the cystic fibrosis in a subject, the compound or composition administered to the subject can comprise a therapeutic moiety that can comprise a targeting moiety that can act as an inhibitor against CAL PDZ.

Further disclosed are methods of using the disclosed compositions to deliver an agricultural product into a plant cell comprising contacting the cell with a peptide as disclosed herein. Compounds that can be delivered to plants include biodefense activators and biostimulants.

### Compositions, Formulations and Methods of Administration

Also disclosed herein are compositions comprising the compounds described herein.

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.10% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The disclosed compositions are bioavailable and can be delivered orally. Oral compositions can be tablets, troches, pills, capsules, and the like, and can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the invention have been described. Nevertheless, it will be understood that various modifications may be made without departing from the spirit and scope of the invention. Accordingly, other embodiments are within the scope of the following claims.

### Examples

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Human Fibronectin Type III (FN3) domain was chosen as the scaffold for Membrane Translocation Domains (MTDs) (Koide, A., et al. (1998) The fibronectin type III domain as a scaffold for novel binding proteins. J. Mol. Biol. 284(4):1141-1151). FN3 is a small (90-100 aa), highly stable protein and has been widely used to develop monobodies that bind to target proteins with high affinity and specificity (Chandler, P. G., et al. (2020) Development and Differentiation in Monobodies Based on the Fibronectin Type 3 Domain. Cells 9(3):610). Previous studies have demonstrated that several loop regions of FN3 are tolerant to mutations (Steven, A., et al. (2012) Design of novel FN3 domains with high stability by a consensus sequence approach, Protein Engineering, Design and Selection. 25(3):107-117). Additionally, FN3 is free of any cysteine or disulfide bond and is thus stable in the intracellular environment. FN3 readily folds into its native form without any physical or chemical assistance and can be produced in Escherichia coli in high yields. Finally, FN3 is derived from an abundant human extracellular protein and is less likely to elicit any immune response.

### Design, Expression, and Purification of MTDs.

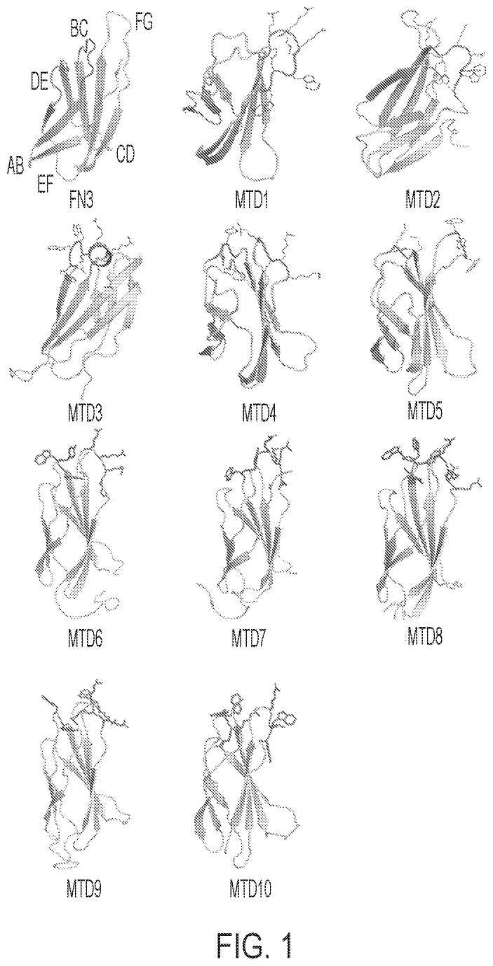

The BC, DE, and FG loops of FN3 have previously shown to be highly tolerant to sequence mutations. The GDSPAS sequence of the FG loop was replaced with RRRWWW (SEQ. ID. NO.:104) to give MTD1 (Table 4). Together with an arginine residue already in the FG loop, this generates a putative CPP motif (R₄W₃) without altering the loop size. Similarly, the tetrapeptide AVTV of the BC loop was replaced with WWWRRR (SEQ. ID. NO.:105) to take advantage of the existing arginine in the loop to form a putative CPP, W₃R₄ (Table 4). The size of the BC loop in the resulting mutant, MTD2, is increased by 2 residues. To explore the possibility of grafting a CPP motif to the other end of FN3, the CPP motif R₄W₃ was substituted for the tripeptide NSP in the CD loop to give MTD3. To test the feasibility of grafting a CPP sequence into two different loops, a relatively hydrophobic tripeptide in the BC loop (VTV) was replaced with WYW and a hydrophilic motif in the FG loop (GDSPAS; SEQ. ID. NO.:106) with RRR to produce MTD4. Finally, MTD5 was generated by switching the WYW and RRR motifs of MTD4. Two mutants, MTD4a and MTD4b, which contain only half of the CPP motif in the BC and FG loops, respectively, were also generated to test the relative importance of the RRRR and WYW motifs. The WYW motif is more hydrophilic than WWW and has previously been reported as the “endosomal escape motif” of cell-permeable antibodies (Kim, J.-S., et al. (2016) Endosomal acidic pH-induced conformational changes of a cytosol-penetrating antibody mediate endosomal escape. J. Control. Rel. 235:165-175). The loop insertion mutants were analyzed by an online program, Phyre2, to predict their folded structures based on homology of sequences. All mutants maintained a similar overall folding to wild type FN3, with the CPP motifs displayed on their surfaces and constrained into the “cyclic” topology (FIG. 1).

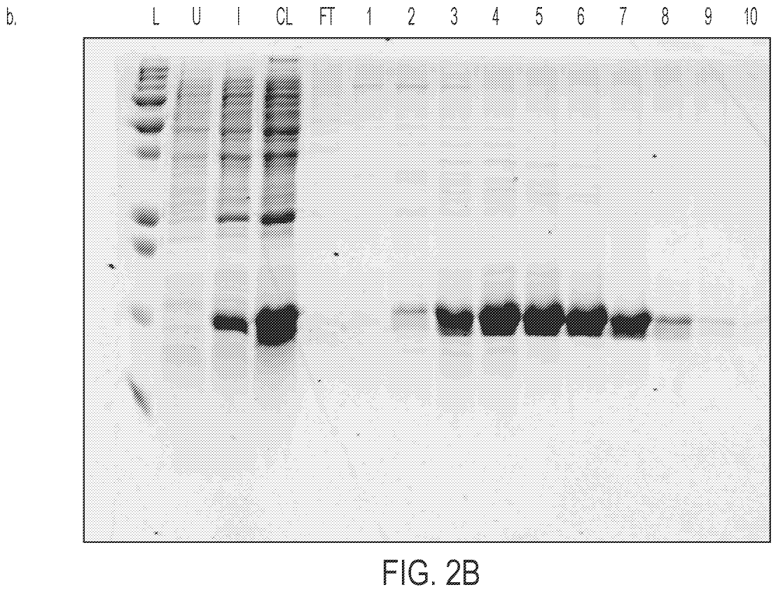

To further improve the properties of MTD4 (e.g., cell entry efficiency, metabolic stability, and expression yield), the BC and FG loops of FN3 were replaced with different combinations of Y, W, A, and R residues to generate MTD6-10 (Table 4). The total cellular entry efficiency of MTD6-10 was assessed by labeling the MTDs at a unique C-terminal cysteine with tetramethylrhodamine-5-maleimide (TMR). HeLa cells were treated with the TMR-labeled proteins (5 μM) for 2 h and analyzed by live cell confocal microscopy. MTD7^{TMR} and MTD9^{TMR} showed similar uptake as MTD4^{TMR}, whereas MTD6^{TMR}, MTD8^{TMR} and MTD10^{TMR} showed much less cellular entry (FIGS. 3A-31). Additionally, the isolated yields for MTD6-10 varied from 0.6 to 6.2 mg/L of E. coli cell culture (Table 4). Note that MTD4 and MTD6 differ only slightly in the BC loop sequence (“WYW” vs “YWW”) and yet have dramatic differences in the isolated yields (9.4 mg/L vs 0.9 mg/L) as well as the cell entry efficiency. Similarly, swapping the CPP motifs between the BC and FG loops of MTD4 resulted in a poorly expressed and much less active variant (MTD5 in Table 4). These results demonstrate that the proper folding/stability and high cellular entry efficiency of the MTDs require not only the presence of amphipathic CPP motifs but also their proper presentation on the protein surface.

TABLE 4
Loop Sequences and Expression Yields of MTDs

| | Insertion | Original Sequence* | Mutant Sequence* | Quality of |
|---|---|---|---|---|
| Protein | Site | (SEQ. ID. NO.) | (SEQ. ID. NO.) | Expression |
| MTD1 | FG loop | TGRGDSPASSKPI (108) | TGRRRRWWWSKPI (111) | No expression |
| MTD2 | BC loop | APAVTVRYY (109) | APWWWRRRRYY (112) | Soluble; yield |
| | | | | 6.4 mg/L |
| MTD3 | CD loop | GGNSPVQE (110) | GGRRRRWWWVQE (113) | Soluble; yield |
| | | | | 2 mg/L |
| MTD4 | BC loop | APAVTVRYY (109) | APAWYWRYY (114) | Soluble; yield |
| | FG loop | TGRGDSPASSKPI (108) | TGRRRRSKPI (115) | 9.4 mg/L |
| MTD5 | BC loop | APAVTVRYY (109) | APARRRRYY (116) | Soluble; yield |
| | FG loop | TGRGDSPASSKPI (108) | TGWYWRSKPI (117) | 0.6 mg/L |
| MTD6 | BC loop | APAVTVRYY (109) | APAYWWRYY (134) | Soluble; yield |
| | FG loop | TGRGDSPASSKPI (108) | TGRRRRSKPI (135) | 0.9 mg/L |
| MTD7 | BC loop | APAVTVRYY (109) | APAWWARYY (136) | Soluble; yield |
| | FG loop | TGRGDSPASSKPI (108) | TGRRRRSKPI (137) | 2.5 mg/L |
| MTD8 | BC loop | APAVTVRYY (109) | APAWWWRRYY (138) | Soluble; yield |
| | FG loop | TGRGDSPASSKPI (108) | TGRRASSKPI (139) | 3.6 mg/L |
| MTD9 | BC loop | APAVTVRYY (109) | APAWWARYY (140) | Soluble; yield |
| | FG loop | TGRGDSPASSKPI (108) | TGWRRRRSKPI (141) | 6.2 mg/L |
| MTD10 | BC loop | APAVTVRYY (109) | APAWWRRYY (142) | Soluble; yield |
| | FG loop | TGRGDSPASSKPI (108) | TGRRWWSKPI (150) | 0.6 mg/L |

*Underlined residues were deleted and bold-faced residues were inserted during
mutagenesis.

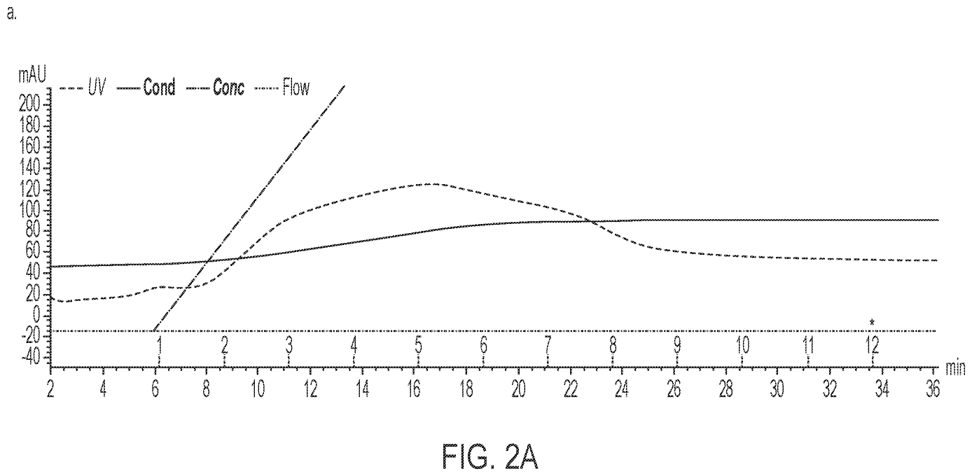

The DNA sequence coding for WT FN3 was chemically synthesized and ligated into prokaryotic expression vector pET-15b. To facilitate protein purification and genetic fusion with cargo proteins, a six-histidine tag and a thrombin cleavage site were added to the N-terminus of FN3, while a flexible linker sequence (GGSGGSGGS; SEQ. ID. NO.: 107) followed by a recognition site for restriction endonuclease SacI and a cysteine was added to its C-terminus (Table 5). All loop insertion mutants were generated by one-step polymerase chain reaction (PCR) method (Qi, D., et al. (2008) A one-step PCR-based method for rapid and efficient site-directed fragment deletion, insertion, and substitution mutagenesis. J. Virol. Meth. 149:85-9020) and expressed in E. coli. Among the mutant proteins, MTD1 failed to produce significant amounts of soluble protein, whereas WT FN3 and MTD2-5 produced soluble proteins in good yields (Table 4). FIGS. 2A-2B show the expression and purification of MTD4 as an example. All proteins were purified to near homogeneity by metal affinity chromatography on a Ni-NTA column.

### Cloning, Expression, and Purification of MTDs.

All loop insertion mutants were generated by one-step polymerase-chain reaction (PCR) method (Qi, D., id.). The peptide sequence (Table 5) for each construct was confirmed by sequencing the entire coding region of the plasmid DNA. Pilot-scale protein expression was carried out to check the levels of expression for Mutant proteins, all mutants were expressed in 5 mL E. coli BL21 (DE3) bacterial culture. The induction was carried out in presence of 0.25 mM IPTG at 37° C. The level of expression was checked by comparing total cell lysate before and after induction on SDS gel (FIGS. 2A-2B).

TABLE 5
Amino Acid Sequences of WT FN3, MTDs, and MTD-Cargo Fusions

| Protein | SEQ ID. NO. | Sequence |
|---|---|---|
| FN3 | 118 | MGSSHHHHHHSSGLVPRGSHMVSDVPRDLEVVAATPTSLLISWDAPAVTVRYYRITYGET |
| | | GGNSPVQEFTVPGSKSTATISGLKPGVDYTITVYAVTGRGDSPASSKPISINYRTGGSGGS |
| | | GGSELC |
| MTD1 | 119 | MGSSHHHHHHSSGLVPRGSHMVSDVPRDLEVVAATPTSLLISWDAPAVTVRYYRITYGET |
| | | GGNSPVQEFTVPGSKSTATISGLKPGVDYTITVYAVTGRRRRWWWSKPISINYRTGGSGG |
| | | SGGSELC |
| MTD2 | 120 | MGSSHHHHHHSSGLVPRGSHMVSDVPRDLEVVAATPTSLLISWDAPWWWRRRRYYRIT |
| | | YGETGGNSPVQEFTVPGSKSTATISGLKPGVDYTITVYAVTGRGDSPASSKPISINYRTGG |
| | | SGGSGGSELC |
| MTD3 | 121 | MGSSHHHHHHSSGLVPRGSHMVSDVPRDLEVVAATPTSLLISWDAPAVTVRYYRITYGET |
| | | GGRRRRWWWVQEFTVPGSKSTATISGLKPGVDYTITVYAVTGRGDSPASSKPISINYRTG |
| | | GSGGSGGSELC |
| MTD4 | 122 | MGSSHHHHHHSSGLVPRGSHMVSDVPRDLEVVAATPTSLLISWDAPAWYWRYYRITYGE |
| | | TGGNSPVQEFTVPGSKSTATISGLKPGVDYTITVYAVTGRRRRSKPISINYRTGGSGGSGG |
| | | SELC |
| MTD4a | 123 | MGSSHHHHHHSSGLVPRGSHMVSDVPRDLEVVAATPTSLLISWDAPAWYWRYYRITYGE |
| | | TGGNSPVQEFTVPGSKSTATISGLKPGVDYTITVYAVTGRGDSPASSKPISINYRTGGSGG |
| | | SGGSELC |
| MTD4b | 124 | MGSSHHHHHHSSGLVPRGSHMVSDVPRDLEVVAATPTSLLISWDAPAVTVRYYRITYGET |
| | | GGNSPVQEFTVPGSKSTATISGLKPGVDYTITVYAVTGRRRRSKPISINYRTGGSGGSGG |
| | | SELC |
| MTD5 | 125 | MGSSHHHHHHSSGLVPRGSHMVSDVPRDLEVVAATPTSLLISWDAPARRRRYYRITYGET |
| | | GGNSPVQEFTVPGSKSTATISGLKPGVDYTITVYAVTGWYWRSKPISINYRTGGSGGSGGS |
| | | ELC |
| MTD6 | 143 | MGSSHHHHHHSSGLVPRGSHMVSDVPRDLEVVAATPTSLLISWDAPAYWWRYYRITYGET |
| | | GGNSPVQEFTVPGSKSTATISGLKPGVDYTITVYAVTGRRRRSKPISINYRTGGSGGSGGSE |
| | | LC |
| MTD7 | 144 | MGSSHHHHHHSSGLVPRGSHMVSDVPRDLEVVAATPTSLLISWDAPAWWARYYRITYGET |
| | | GGNSPVQEFTVPGSKSTATISGLKPGVDYTITVYAVTGRRRRSKPISINYRTGGSGGSGGSE |
| | | LC |
| MTD8 | 145 | MGSSHHHHHHSSGLVPRGSHMVSDVPRDLEVVAATPTSLLISWDAPAWWWRRYYRITYGE |
| | | TGGNSPVQEFTVPGSKSTATISGLKPGVDYTITVYAVTGRRASSKPISINYRTGGSGGSGGS |
| | | ELC |
| MTD9 | 146 | MGSSHHHHHHSSGLVPRGSHMVSDVPRDLEVVAATPTSLLISWDAPAWWARYYRITYGET |
| | | GGNSPVQEFTVPGSKSTATISGLKPGVDYTITVYAVTGWRRRRSKPISINYRTGGSGGSGG |
| | | SELC |
| MTD10 | 147 | MGSSHHHHHHSSGLVPRGSHMVSDVPRDLEVVAATPTSLLISWDAPAWWRRYYRITYGET |
| | | GGNSPVQEFTVPGSKSTATISGLKPGVDYTITVYAVTGRRWWSKPISINYRTGGSGGSGGS |
| | | ELC |
| MTD4- | 126 | MGSSHHHHHHSSGLVPRGSHMVSDVPRDLEVVAATPTSLLISWDAPAWYWRYYRITYGE |
| PTP1B | | TGGNSPVQEFTVPGSKSTATISGLKPGVDYTITVYAVTGRRRRSKPISINYRTGGSGGSGG |
| | | SELMEMEKEFEQIDKSGSWAAIYQDIRHEASDFPCRVAKLPKNKNRNRYRDVSPFDHSRIK |
| | | LHQEDNDYINASLIKMEEAQRSYILTQGPLPNTCGHFWEMVWEQKSRGVVMLNRVMEKGS |
| | | LKCAQYWPQKEEKEMIFEDTNLKLTLISEDIKSYYTVRQLELENLTTQETREILHFHYTTWPD |
| | | FGVPESPASFLNFLFKVRESGSLSPEHGPVVVHCSAGIGRSGTFCLADTCLLLMDKRKDPS |
| | | SVDIKKVLLEMRKFRMGLIQTADQLRFSYLAVIEGAKFIMGDSSVQDQWKELSHEDLEPPPE |
| | | HIPPPPRPPKRILEPHN |
| MTD4- | 127 | MGSSHHHHHHSSGLVPRGSHMVSDVPRDLEVVAATPTSLLISWDAPAWYWRYYRITYGE |
| NS1 | | TGGNSPVQEFTVPGSKSTATISGLKPGVDYTITVYAVTGRRRRSKPISINYRTGGSGGSGG |
| | | SELGSVSSVPTKLEVVAATPTSLLISWDAPAVTVDYYVITYGETGGNSPVQKFEVPGSKST |
| | | ATISGLKPGVDYTITVYAWGWHGQVYYYMGSPISINYRT |
| MTD4- | 128 | MGSSHHHHHHSSGLVPRGSHMVSDVPRDLEVVAATPTSLLISWDAPAWYWRYYRITYGE |
| RBDV | | TGGNSPVQEFTVPGSKSTATISGLKPGVDYTITVYAVTGRRRRSKPISINYRTGGSGGSGG |
| | | SELKTSNTIRVLLPNQEWTVVKVRNGMSLHDSLMKALKRHGLQPESSAVFRLLHEHKGKK |
| | | ARLDWNTDAASLIGEELQVDFLDHVPLTTHNFARKTFLKLGIHRD |
| MTD4- | 148 | MGSSHHHHHHSSGLVPRGSHMVSDVPRDLEVVAATPTSLLISWDAPAWYWRYYRITYGET |
| SEP | | GGNSPVQEFTVPGSKSTATISGLKPGVDYTITVYAVTGRRRRSKPISINYRTGGSGGSGGSE |
| | | LSKGEELFTGVVPILVELDGDVNGHKFSVSGEGEGDATYGKLTLKFICTTGKLPVPWPTLVTT |
| | | LTYGVQCFSRYPDHMKRHDFFKSAMPEGYVQERTIFFKDDGNYKTRAEVKFEGDTLVNRIEL |
| | | KGIDFKEDGNILGHKLEYNYNDHQVYIMADKQKNGIKANFKIRHNIEDGGVQLADHYQQNTPIG |
| | | DGPVLLPDNHYLFTTSTLSKDPNEKRDHMVLLEFVTAAGITHGMDELYK |
| MENC | 149 | MGSSHHHHHHSSGLVPRGSHMVSDVPRDLEVVAATPTSLLISWDAPAWYWRYYRITYGET |
| | | GGNSPVQEFTVPGSKSTATISGLKPGVDYTITVYAVTGRRRRSKPISINYRTGGSGGSGGSE |
| | | LMVSKGEELFTGVVPILVELDGDVNGHKFSVSGEGEGDATYGKLTLKFICTTGKLPVPWPTL |
| | | VTTLTYGVQCFSRYPDHMKQHDFFKSAMPEGYVQERTIFFKDDGNYKTRAEVKFEGDTLVN |
| | | RIELKGIDFKEDGNILGHKLEYNYNSHNVYIMADKQKNGIKVNFKIRHNIEDGSVQLADHYQQN |
| | | TPIGDGPVLLPDNHYLSTQSALSKDPNEKRDHMVLLEFVTAAGITLGMDELYKSGEQKLISEED |
| | | LGGPKKKRKVSNLLTVHQNLPALPVDATSDEVRKNLMDMFRDRQAFSEHTWKMLLSVCRSW |
| | | AAWCKLNNRKWFPAEPEDVRDYLLYLQARGLAVKTIQQHLGQLNMLHRRSGLPRPSDSNAV |
| | | SLVMRRIRKENVDAGERAKQALAFERTDFDQVRSLMENSDRCQDIRNLAFLGIAYNTLLRIAEI |
| | | ARIRVKDISRTDGGRMLIHIGRTKTLVSTAGVEKALSLGVTKLVERWISVSGVADDPNNYLFCR |
| | | VRKNGVAAPSATSQLSTRALEGIFEATHRLIYGAKDDSGQRYLAWSGHSARVGAARDMARAG |
| | | VSIPEIMQAGGWTNVNIVMNYIRNLDSETGAMVRLLEDGD |

The large-scale expression conditions were the same as used for small scale, E. coli cells were centrifuged and stored at −80° C. These cells were lysed using lysis buffer (50 mL of wash buffer, 0.2 mg/mL lysozyme, 2 mM β-mercaptoethanol, 2 mM PMSF, 2 tablets of Roche complete protease inhibitor cocktail). After homogeneously resuspending the cell palate in lysis buffer the cells were sonicated (Amp. 70%) twice. The crude cell lysate was centrifuged (12000 g for 20 min) and the soluble cell lysate was collected. Protein purification was carried out by using fast protein liquid chromatography (FPLC) and the soluble cell lysate was loaded onto a Ni-NTA column (with 15 mM imidazole). The column was exhaustively washed with wash buffer (50 mM Tris, pH 7.4, 300 mM NaCl, 5% glycerol and 50 mM imidazole). Protein was eluted with wash buffer containing a linear gradient of 50-500 mM imidazole (pH 7.4) over 30 min.

### Cloning, Expression, and Purification of MTD4-PTP1B, MTD4-NS1, MTD4-RBDV, MTD4-SEP, MTD4-GFP11 and MENC.

The coding sequence of PTP1B (amino acids 1-321) was amplified by PCR using plasmid DNA as template and primers containing SacI and BamHI restriction sites at the 5′ and 3′ terminus of the PTP1B coding sequence, respectively. The PCR product was digested with restriction enzymes SacI and BamHI and ligated into plasmid pET-15b-MTD4 linearized with the same two enzymes. This resulted in the fusion of PTP1B to the C-terminus of MTD4. Other pET15b-based plasmids encoding MTD4-RBDV, MTD4-NS1, MTD4-SEP, and MENC fusion proteins, were similarly constructed, except that a XhoI restriction site was added to the 3′ end of RBDV, NS1, SEP and ENC coding sequences instead of BamHI. GFP11 peptide was inserted to the C-terminus of MTD4 by one-step PCR reaction (D. Qi, et al., “A one-step PCR-based method for rapid and efficient site-directed fragment deletion, insertion, and substitution mutagenesis,” J Virol Methods, 149(1):85-90, 2008). The authenticity of the DNA constructs was confirmed by restriction mapping and sequencing of the entire coding sequences.

Escherichia coli BL21 (DE3) cells transformed with the proper plasmid were grown in LB medium supplemented with 75 mg/L ampicillin at 37° C. When OD₆₀₀ reached 0.6, the cells were induced by the addition of 0.25 mM IPTG for 4 h at 37° C. The cells were pelleted by centrifugation. For MTD4-RBDV, the cell pellet was resuspended in 50 mL (for each liter of cell culture) of lysis buffer [50 mM Tris (pH 7.4), 150 mM NaCl, 25 mM Imidazole, 3 mM β-mercaptoethanol, protease inhibitor cocktail tablets, and 20 mg/ml lysozyme]. The cells are briefly sonicated and centrifuged at 12,000 g for 20 min. The crude lysate was loaded onto a 5-mL Histrap Ni-NTA column attached to FPLC. The column was exhaustively washed with wash buffer (50 mM Tris, pH 7.4, 300 mM NaCl, 5% glycerol and 50 mM imidazole). Protein was eluted with wash buffer containing 500 mM imidazole. MTD4-PTP1B, MTD4-SEP, MENC, MTD4-GFP11 and MTD4-NS1 were similarly purified, except that for MTD4-NS1 the cell pellet was resuspended in 50 mM Tris (pH 8), 500 mM NaCl, 25 mM Imidazole, 3 mM β-mercaptoethanol, protease inhibitor cocktail tablets, and 20 mg/ml lysozyme. MENC protein was expressed in BL21 Rosetta pLysS cell strain and the induction was performed at 18° C. overnight. The specific activity of MTD4-PTP1B was determined by using p-nitrophenyl phosphate (pNPP) as substrate and was found to be similar to that of WT PTP1B.

### Cellular Entry Efficiency of MTDs.

Wild type FN3 and the MTDs were fluorescently labeled at their single C-terminal cysteine with tetramethylrhodamine-5-maleimide (TMR). HeLa (human cervical cancer) cells were incubated with the TMR-labeled proteins and imaged without fixation by confocal microscopy. Interestingly, WT FN3 showed significant cell entry, although the intracellular fluorescence pattern is punctate, indicating that most of the internalized protein is entrapped inside the endosome/lysosome (FIG. 3A). In contrast, HeLa cells treated with MTD4^{TMR} exhibited readily visible diffuse fluorescence throughout the cell volume (including the nucleus), in addition to punctate fluorescence (FIG. 3B). The presence of diffuse intracellular fluorescence indicates that a significant fraction of the internalized MTD4^{TMR} has exited the endosome and successfully reached the cytosol (and nucleus). The fluorescence pattern of MTD2^{TMR}-treated cells is somewhere in between those of FN3- and MTD4-treated cells; while some diffuse fluorescence is visible, the fluorescence is predominantly punctate (FIG. 3B). MTD5^{TMR} did not produce significant intracellular fluorescence (data not shown).

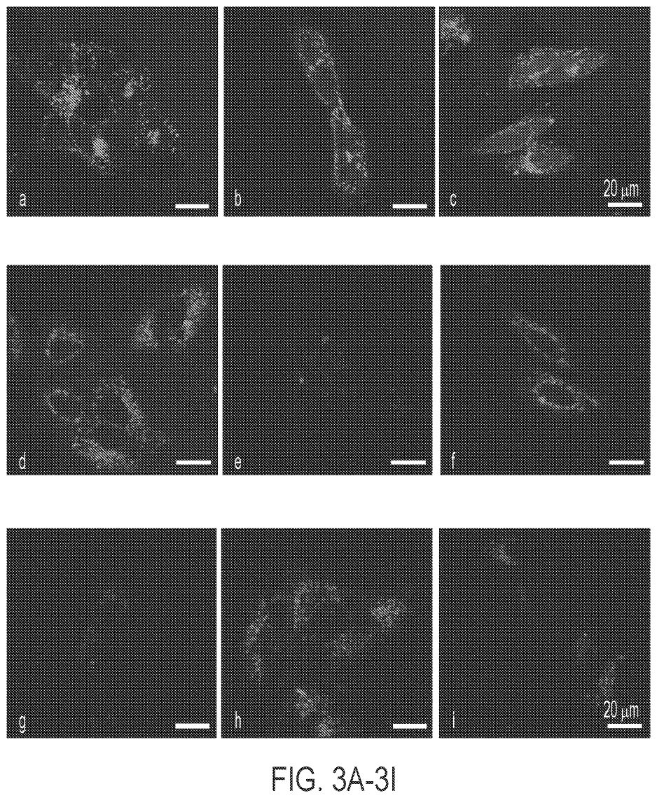

To quantify the cellular entry efficiency, the cells were next analyzed by flow cytometry and compared the results with that of CPP12, a highly efficient cyclic CPP previously reported. The preliminary data, after adjustment of the degree of dye-labelling of the protein, showed that MTD4 entered HeLa cells more efficiently than FN3 or MTD2 but less than CPP12 (FIG. 4).

### Intracellular Delivery of PTP1B.

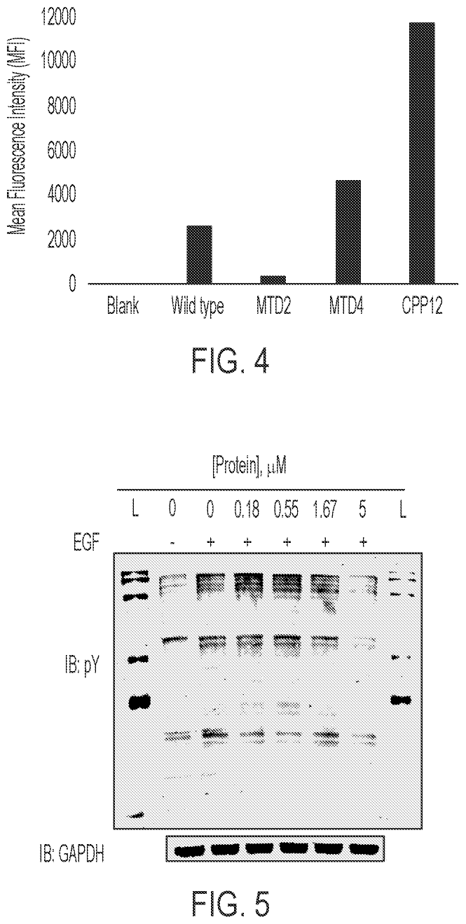

To demonstrate functional delivery of protein cargos into the cytosol of mammalian cells by the MTDs, protein tyrosine phosphatase 1B (PTP1B) was chosen as cargo and genetically fused it to the C-terminus of MTD4. Tyrosyl phosphorylation is generally limited to cytosolic and nuclear proteins or the cytosolic domains of transmembrane proteins. PTP1B is a broad-specificity phosphatase that catalyzes the dephosphorylation of many intracellular proteins (Selner, N. G., et al. (2014) Diverse levels of sequence selectivity and catalytic efficiency of protein-tyrosine phosphatases. Biochem. 53(2):397-412). Cytosolic delivery of PTP1B is expected to decrease the phosphotyrosine (pY) level of intracellular proteins, which can be readily monitored by anti-pY Western blotting. NIH3T3 cells were treated with different concentrations of MTD4-PTP1B for 4 h, washed, and lysed in the presence of protease and phosphatases inhibitors. The cellular proteins were separated by SDS-PAGE, transferred to a nitrocellulose membrane, and blotted with anti-pY antibody 4G10. MTD4-PTP1B caused dose-dependent reduction of pY levels in HeLa cells and nearly complete loss of pY at 5 μM (FIG. 5).

### Intracellular Delivery of Ras Inhibitors.

To further demonstrate the utility of MTD4 and generate cell-permeable proteins of therapeutic potential, MTD4 was fused to two previously reported proteins that bind to mutant KRas with high affinity and specificity. Mutations in Ras (including K-, H-, and NRas) are found in ~30% all human cancers, making Ras one of the most important cancer drug targets (Prior, I. A., et al. (2012) A comprehensive survey of Ras mutations in cancer. Cancer Res. 72(10):2457-67; Khan, I., et al. (2020) Therapeutic targeting of RAS: New hope for drugging the “undruggable. Biochim. Biophys. Acta, Mol. Cell Res. 1867, 118570). Unfortunately, Ras is one of the most challenging (and undruggable) drug targets, because it is intracellular and its surface has no major binding pocket for small molecules to bind. To date, several small molecules that covalently modify (and inhibit) the G12C mutant KRas have entered the clinic and one has been approved by the FDA, validating Ras as a viable target for treating Ras mutant cancers (Moore, A. R., et al. (2020) RAS-targeted therapies: is the undruggable drugged? Nature Rev. Drug Disc. 19(8):533-552). A potent, noncovalent inhibitor selective for KRas G12D mutant was also recently reported (Wang, X., et al. (2021) Identification of MRTX1133, a Noncovalent, Potent, and Selective KRASG12D Inhibitor. J. Med. Chem. 10.1021/acs.jmedchem.1c01688). However, no such agent exists for many other Ras mutants (e.g., G12V, G12S, G13C, and Q61H).

Previous investigators have generated potent protein inhibitors of KRas by phage display screening. Wiechmann et al. screened a phage-displayed library of C-Raf Ras-binding domain (RBD) and identified several variants, RBDVs, which bind to the effector-binding site of GTP-bound HRas with ~20-fold higher affinity than WT C-Raf RBD (K_{D}~3 nM) (Wiechmann, S., et al. (2020) Conformation-specific inhibitors of activated Ras GTPases reveal limited Ras dependency of patient-derived cancer organoids. J. Biol. Chem. 295(14):4526-4540). Expression of the RBDVs in Ras mutant cancer cells blocked Ras signaling and resulted in apoptosis of the cancer cells. Similarly, Koide and co-workers screened a large library of phage-displayed FN3 mutants against HRas and discovered a potent binder, termed “NS1”, which binds to an allosteric site and prevent Ras dimerization on the plasma membrane (Spencer-Smith, R., et al. (2017) Inhibition of RAS function through targeting an allosteric regulatory site. Nat. Chem. Biol. 13:62-68). When expressed inside the cell, NS1 also inhibited Ras signaling and induced apoptotic death of Ras mutant cancer cells (Khan, I., et al. (2019) Targeting the α4-α5 dimerization interface of K-RAS inhibits tumor formation in vivo. Oncogene 38(16):2984-2993). Unlike the RBDVs, which bind to all Ras isoforms (K-, H-, and NRas) in their GTP-bound state, NS1 is specific for HRas (K_{D}=13 nM) and KRAS (K_{D}=65 nM), irrespective of the nucleotide identity. Unfortunately, RBDVs and NS-1 are not suitable as therapeutic agents because they cannot penetrate the cell membrane to reach the target protein.

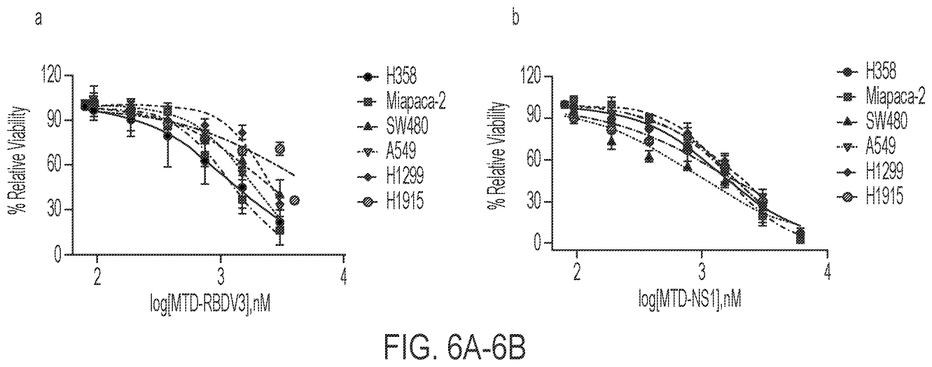

One of the RBDVs (RBDV3) or NS1 was genetically fused to the C-terminus of MTD4 to produce MTD4-RBDV and MTD4-NS1, respectively. The proteins were expressed in E. coli and purified to near homogeneity by metal affinity chromatography. The fusion proteins were next tested for their ability to reduce the viability of Ras mutant cancer cells. MTD4-RBDV dose-dependently reduced the viability of non-small cell lung cancer (H358), pancreas ductal adenocarcinoma (Mia PaCa-2), non-small cell lung cancer (A549) and colorectal cancer cells (SW480) with IC₅₀ values of 1.2±0.2, 1.2±0.1, 1.7±0.2, and 2.2±0.4 μM respectively (FIG. 6A). Similarly, MTD4-NS1 dose-dependently reduced the viability of aforementioned cells with IC₅₀ values of 1.4±0.2, 1.5±0.1, 1.8±0.1, and 0.9±0.2 μM, respectively (FIG. 6B). To verify the selectivity of NS1 toward KRas and HRas not NRas, both fusion proteins in H1915 a non-small cell lung carcinoma cells (HRAS^{Q61L}) mutant and H1299 non-small cell lung carcinoma cells (NRAS^{Q61K}) mutant were tested. MTD4-RBDV showed activity in both cells with an IC₅₀ of 4.7±1.1 μM and IC₅₀ of 2.4±0.3 μM, respectively. Surprisingly, MTD4-NS1 showed activity in both cells with an IC₅₀ of 1.5±0.2 μM and IC₅₀ of 1.6±0.2 μM. NS1 was fused to the N-terminus of MTD4 to generate NS1-MTD4. NS1-MTD4 also dose dependently decreased the viability of H358 and MiaPaCa-2 with IC₅₀ of 1.1±0.1 μM and IC₅₀ of 1.8±0.2 μM, respectively (FIG. 6C). NS1-MTD4 also reduced the viability of H1299 cells with an IC₅₀ 1.6±0.2 μM.

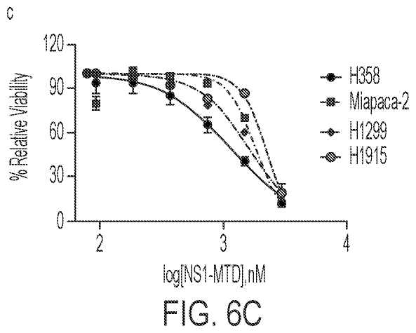

To determine whether the loss of cancer cell viability is caused by on-target inhibition of the Ras-effector protein interaction and Ras signaling, a bioluminescence resonance energy transfer (BRET) assay developed by Rabbitts and co-workers was used (Bery, N., et al. (2018) BRET-based RAS biosensors that show a novel small molecule is an inhibitor of RAS-effector protein-protein interactions. eLife 7:e37122). Human embryonic kidney (HEK293T) cells, which do not carry any Ras mutation and are relatively insensitive to Ras inhibitors, were transfected with plasmid DNAs encoding KRas G12V (or G12D)-luciferase and c-Raf RBD-green fluorescent protein (GFP) fusions. Interaction between the KRas mutants and the RBD results in a BRET signal from the luciferase donor to the GFP receptor, upon the addition of a membrane-permeable luciferase substrate, coelenterazine 400a. Inhibitors that block the Ras-RBD interaction are expected reduce the BRET signal. As shown in FIG. 7, MTD4-RBDV dose-dependently decreased the BRET signal in HEK293T cells transfected with either the KRas G12V or G12D mutant, with IC₅₀ values of 5-10 μM. MTD4-NS1 also reduced the BRET signal but was less potent than MTD4-RBDV, which is in a good agreement with their relative Ras-binding affinities. In addition, unlike RBDV, NST does not bind to the effector-binding site and does not directly compete with Raf for binding to HRas or KRas. Instead, NS1 binds to the dimerization site of HRas/KRas and inhibits the Ras-Raf interaction indirectly. Note that for MTD4-NS1, there was a sudden drop in the BRET ratio at 10 μM protein; this is likely caused by partial loss of HEK293T cell viability at high inhibitor concentrations. The latter again suggests that in addition to on-target inhibition of Ras signaling, MTD4-NS1 may have off-target effects as well.

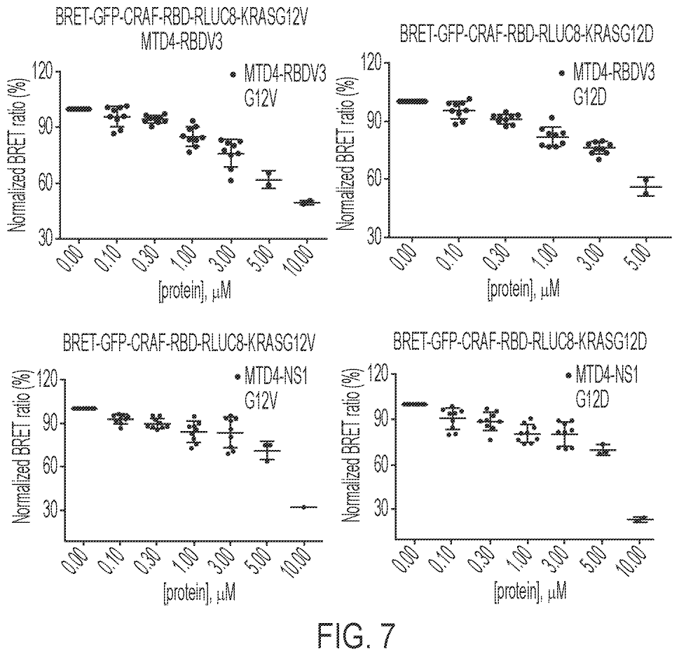

The on-target activity of the fusion proteins was further assessed by examining the phosphorylation levels of signaling proteins downstream of Ras by Western blot analysis. Ras activates the Raf/MEK/ERK and PI3K/Akt signaling pathways and increases the phosphorylation of protein kinases MEK, ERK, and Akt. Inhibition of Ras function should decrease the phosphorylation levels of Akt and MEK. Indeed, treatment of MiaPaCa-2 cells with MTD4-RBDV dose-dependently reduced the phosphorylation of Akt and MEK with IC₅₀ values of 1-3 μM, whereas the total Akt and MEK levels remained relatively constant (FIG. 8A). MTD4-NS1 also decreased the p-Akt and p-MEK levels but was less potent than MTD4-RBDV (FIG. 8B).

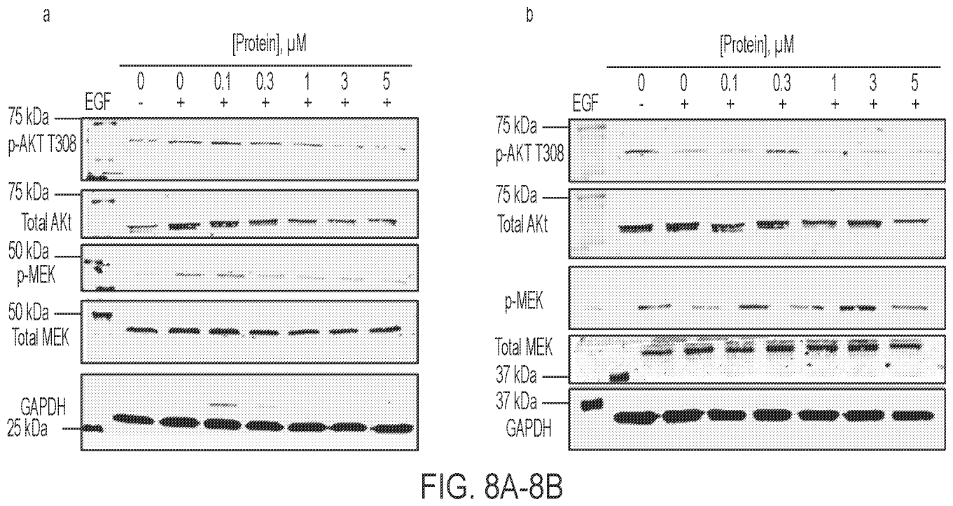

Whether MTD4-RBDV and MTD4-NS1 induce apoptosis of Ras mutant cancer cells was tested. Thus, H358 lung cancer cells were treated with the fusion proteins for 24 h and the stained with Alexa Fluor™ 488-annexin V and propidium iodide prior to flow cytometry analysis. MTD4-RBDV showed a dose-dependent increase in the Annexin V-positive cell population indicating that apoptosis is the cause of the viability loss observed in the Cell Glo assay (FIG. 9). MTD4-NS1 also caused robust apoptosis at concentration as low as 2.5 μM (FIG. 9).

### Cytosolic Delivery Efficiency of MTD4.

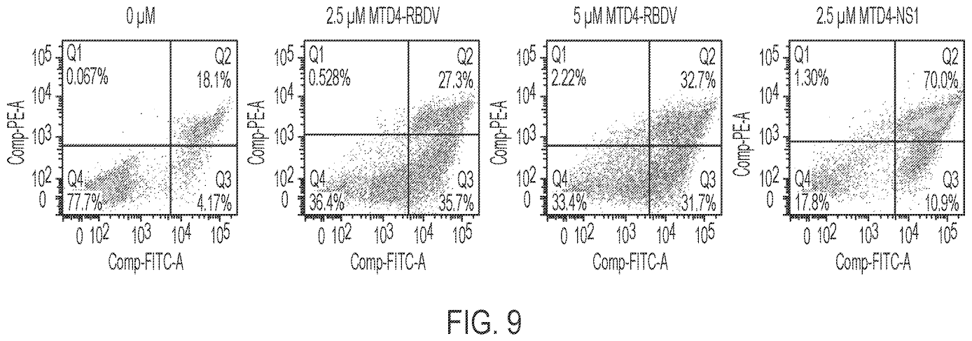

Out of the 10 MTDs generated, MTD4 has relatively high yield of expression in E. coli and excellent total cellular entry efficiency as monitored by confocal microscopy. MTD4 was thus selected for further evaluation and determined its cytosolic delivery efficiency by using two different methods. First, a green fluorescent protein (GFP) complementation assay was used, which involves the binding of a 16-aa peptide GFP11 (which corresponds to the 11^{th} β-strand of GFP) to superfolder GFP1-10 to form a functional GFP. GFP11 was genetically fused to the C-terminus of MTD4. As a comparison, GFP11 was also chemically conjugated to CPP12. HEK293T cells were transiently transfected to express GFP1-10 protein, incubated for 6 h with 10 μM GFP11, MTD4-GFP11, or CPP12-GFP11, and examined by live cell confocal microscopy. Cells treated with MTD4-GFP11 exhibited strong and diffuse fluorescence throughout the cell volume (FIGS. 10A-10D). Cells treated with CPP12-GFP11 also showed strong fluorescence although the signals were more punctate. In contrast, untreated cells and cells treated with unconjugated GFP11 showed little fluorescence.

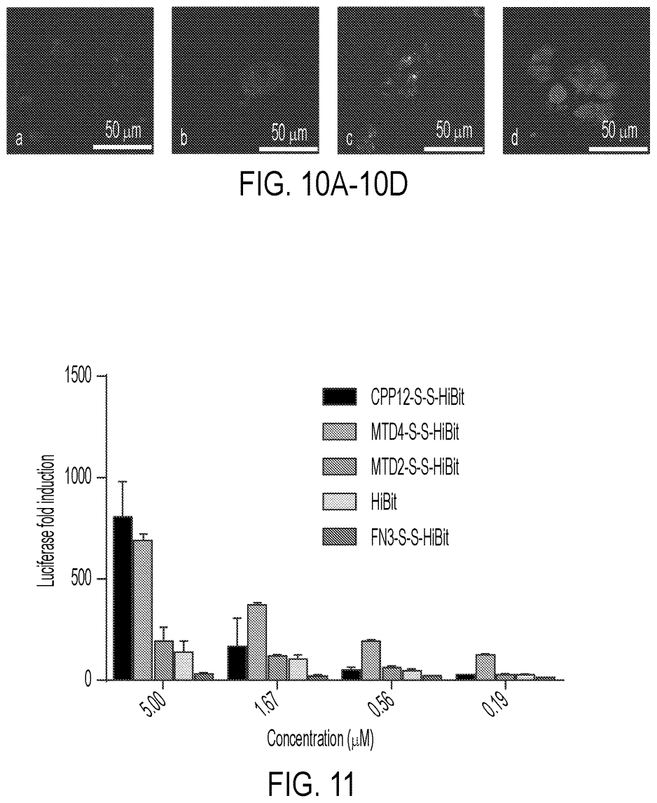

A previously reported luciferase complementation assay was employed (S. L. Y. Teo, J et al., “Unravelling cytosolic delivery of cell penetrating peptides with a quantitative endosomal escape assay,” Nat Commun, 12(1):3721, 2021) by conjugating HiBit, an 11-residue peptide derived from NanoLuc (VSGWRLFKKIS) (SEQ ID NO: 151), to FN3, MTDs and CPP12 via a disulfide linkage. HEK293T cells were transfected with a plasmid DNA coding for an 18-kDa subunit of NanoLuc (LgBit) and then incubated with HiBit or the HiBit conjugates. Upon successful delivery into the cytosol, the HiBit peptide is released from the conjugates by intracellular thiols (e.g., glutathione) and binds specifically to the cytosolic LgBit to form a catalytically active luciferase, whose activity is quantitated in real time by the addition of a cell-permeable substrate furimazine. CPP12-S—S-HiBit, MTD4-S—S-HiBit, and MTD2-S—S-HiBit dose dependently increased the luciferase activity in HEK293T cells, whereas FN3-S—S-HiBit did not (FIG. 11). Interestingly, MTD4 and CPP12 showed similar cytosolic delivery efficiencies at high concentrations (e.g., 5 μM); however, at lower concentrations (e.g., 0.19 and 0.56 μM) MTD4 was 5-10-fold more active than CPP12 (FIG. 10A-10D). The unconjugated HiBit also resulted in some luciferase activity at high concentrations, likely because HiBit has weak intrinsic cell-penetrating activity given its amphipathic sequence (M. K. Schwinn et al., “CRISPR-Mediated Tagging of Endogenous Proteins with a Luminescent Peptide,” ACS Chem Biol, 13(2):467-474, 2018).

### Intracellular Delivery of Additional Protein Cargos.

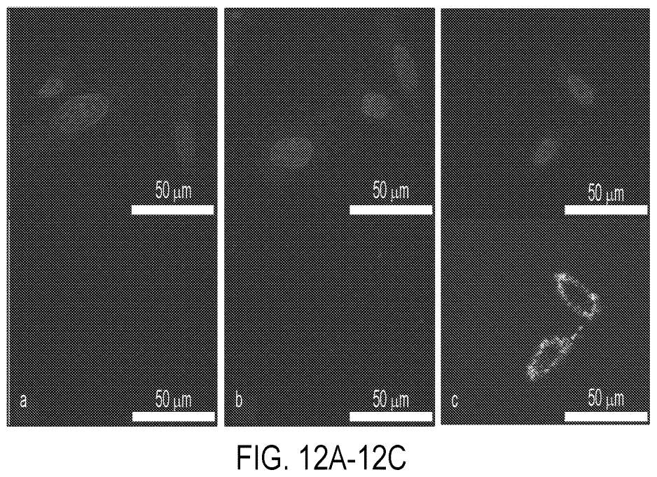

MTD4 was first tested for its ability to deliver a protein cargo, Superecliptic pHluorin (SEP). SEP is a pH sensitive variant of GFP (pKa ~7.2); it is highly fluorescent in the neutral environment of the mammalian cytosol (pH 7.4) but essentially non-fluorescent inside the endosome (pH 5.5-6.5) or lysosome (pH 4.5-5.5) (S. Sankaranarayanan, et al., “The use of pHluorins for optical measurements of presynaptic activity,” Biophys J, 79(4):2199-208, 2000). Therefore, any intracellular fluorescence reflects largely the amount of SEP that has successfully reached the cytosol. SEP was genetically fused to the C-terminus of MTD4 and purified the MTD4-SEP fusion protein from E. coli. HeLa cells were incubated with 5 μM SEP or MTD4-SEP for 2 h and imaged by live-cell confocal microscopy. Cells treated with MTD4-SEP showed intense fluorescence throughout the cell volume, whereas the untreated cells or cells treated with SEP had no detectable fluorescence (FIGS. 12A-12C).

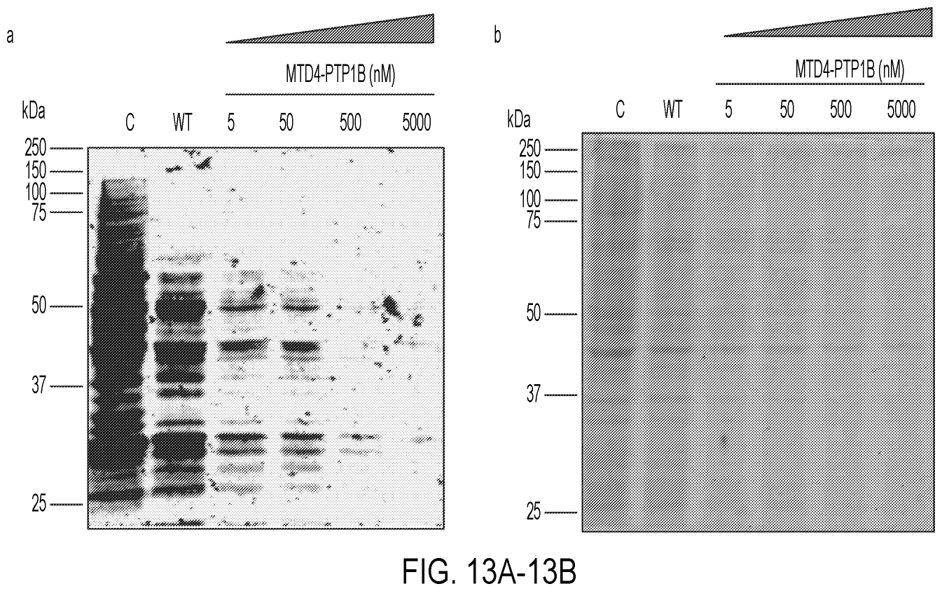

To demonstrate functional delivery of protein cargos into the cytosol of mammalian cells, protein tyrosine phosphatase 1B (PTP1B) was chosen as cargo and genetically fused it to the C-terminus of MTD4. Tyrosyl phosphorylation is generally limited to cytosolic and nuclear proteins or the cytosolic domains of transmembrane proteins. PTP1B is a broad-specificity phosphatase that catalyzes the dephosphorylation of many intracellular proteins (N. G. Selner et al., “Diverse levels of sequence selectivity and catalytic efficiency of protein-tyrosine phosphatases,” Biochemistry, 53(2):397-412, 2014). Cytosolic delivery of PTP1B is expected to decrease the phosphotyrosine (pY) level of intracellular proteins, which can be readily monitored by anti-pY Western blotting. HEK293T cells were treated with different concentrations of MTD4-PTP1B for 6 h, washed, and lysed in the presence of protease and phosphatases inhibitors. The cellular proteins were separated by SDS-PAGE, transferred to a nitrocellulose membrane, and blotted with anti-pY antibody 4G10. MTD4-PTP1B dose-dependently reduced the global pY levels in HEK293T cells with an EC₅₀ value of ≤5 nM (FIGS. 13A-13B). Based on the fact that the cells treated with 5 nM MTD4-PTP1B contained lower pY levels than those treated with 5 μM unconjugated PTP1B (WT), MTD4 was estimated to increase the cytosolic entry of PTP1B by >1000-fold.

### In-Vivo Biodistribution of MTD4-EGFP-NLS-Cre.

To study the biodistribution of MTD4 in mice, EGFP-NLS-Cre protein was genetically fused to the C terminus of MTD4. Cre recombinase is an enzyme derived from P1 bacteriophage and catalyzes site-specific DNA recombination (K. Abremski, et al., “Bacteriophage P1 site-specific recombination. Purification and properties of the Cre recombinase protein,” J Biol Chem, 259(3):1509-14, 1984). Successful delivery of the MTD4-EGFP-NLS-Cre (MENC) fusion protein into the cells of a transgenic mouse is expected to cause a DNA recombination event that activates the expression of a red fluorescent protein (mCherry). Treatment of the primary cells derived from the transgenic mouse in vitro with 1 μM MENC resulted in mild expression of mCherry after 48 h (data not shown).

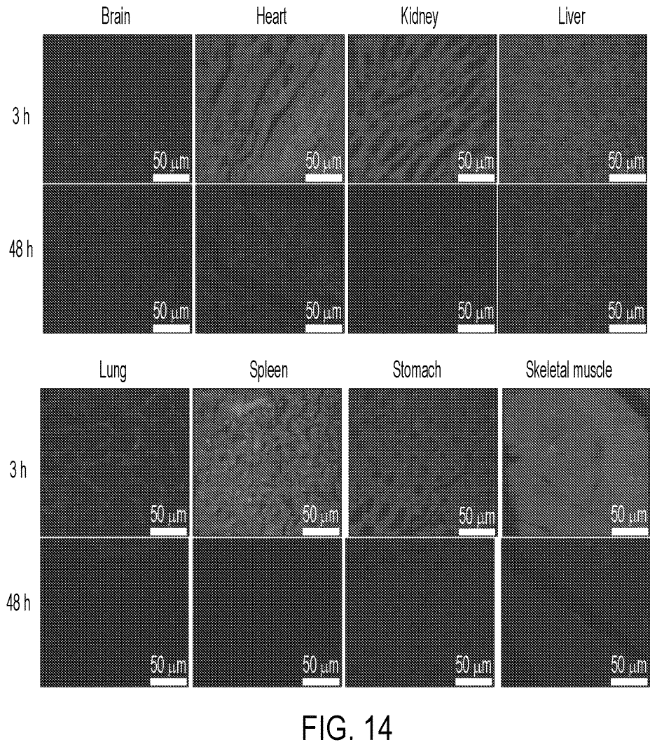

Encouraged by the in vitro data, MENC (8 mg/kg) was injected into the tail vein of 4 transgenic mice; 2 mice were euthanized after 3 h while the others 48 h post treatment. Different organs were harvested, fixed, embedded, and sliced for visualization under a confocal microscope. The samples were treated with true black dye to reduce any autofluorescence from the tissues. Strong EGFP fluorescence was observed in most of the tissues except for the brain, indicating that MENC is broadly biodistributed into different organs (FIG. 14). On the other hand, the mCherry signal was weak in most organs even after 48 h, likely because the nuclear localization sequence (NLS) is flanked by two protein domains and has poor nuclear localization efficiency. Nevertheless, the confocal images clearly demonstrate the biodistribution of MENC in different mouse organs.

### Serum Stability of MTD4.

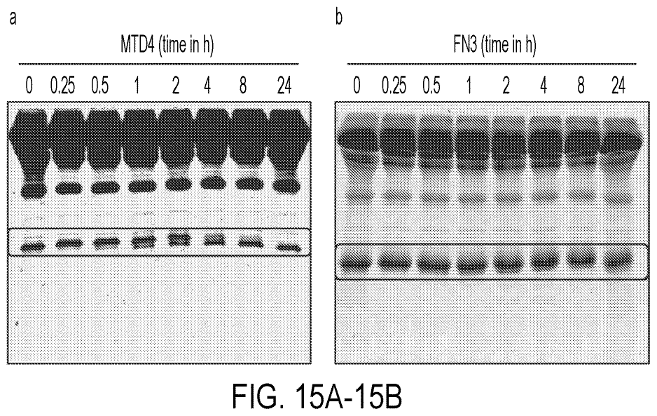

MTD4 was incubated with human serum for varying periods of time and analyzed by SDS-PAGE. MTD4 appears to undergo proteolysis at a single site near the C-terminus, as the cleavage product is only slightly smaller than MTD4 and capable of binding to a nickel-affinity column via its N-terminal 6×His tag (FIG. 15A). Degradation of MTD4 occurs with a t_{1/2} of ~3 h and is mostly complete after 24 h. As a comparison, FN3 appears to undergo a similar cleavage but with a t_{1/2} of >24 h (FIG. 15B). Mass spectrometric analyses confirmed the cleavage of FN3 and MTD4.

### Other Suitable MTD Scaffolds.

Although the current MTDs are all derived from the 10th FN3 domain of human fibronectin, it is hypothesized that that many other protein domains may also serve as appropriate scaffolds for engineering additional MTDs. Generally, a good scaffold should contain two or more adjacent surface loops (for incorporation of two CPP motifs), be free of disulfides and yet stably folded (to tolerate sequence changes), be expressed in E. coli or eukaryotic hosts with high yield and have no biological function of its own. Based on these criteria, examples of suitable MTD scaffolds include other FN3 domains of fibronectin (Komblihtt A R, et al., Primary structure of human fibronectin: differential splicing may generate at least 10 polypeptides from a single gene. EMBO J. 4(7):1755-1759, 1985), nanobodies (Muyldermans S., “Nanobodies: natural single-domain antibodies.” Annu Rev Biochem. 82:775-797, 2013), designed ankyrin repeat proteins (DARPins) (Stumpp M T, et al., “DARPins: a true alternative to antibodies.” Curr Opin Drug Discov Devel. 10(2):153-159, 2007), consensus tetratricopeptide repeats (CTPRs) (Uribe K B, et al., “Engineered Repeat Protein Hybrids: The New Horizon for Biologic Medicines and Diagnostic Tools.” Acc Chem Res. 54(22):4166-4177, 2021), Anticalins (Rothe C, et al., Anticalin™ Proteins as Therapeutic Agents in Human Diseases. BioDrugs. 32(3):233-243, 2018), nanofitins/affitins (Goux M, et al., “Nanofitin as a New Molecular-Imaging Agent for the Diagnosis of Epidermal Growth Factor Receptor Over-Expressing Tumors.” Bioconjug Chem. 28(9):2361-2371, 2017), Affimers (Tiede C, et al., “Affimer proteins are versatile and renewable affinity reagents.” Elife. 6:e24903, 2017). Affilins (Ebersbach H, et al., “Affilin-novel binding molecules based on human gamma-B-crystallin, an all beta-sheet protein.” J Mol Biol. 372(1):172-185, 2007), FHA domains (Durocher D, et al., “The FHA domain.” FEBS Lett. 513(1):58-66, 2002), and SH2 domains (Pawson T, et al., “SH2 domains, interaction modules and cellular wiring.” Trends Cell Biol. 11(12):504-511, 2001).

### Assays

### Confocal Microscopy.

HeLa cells were seeded in a 35/10 mm glass-bottomed microwell dish with four compartments at a density of 5×10⁴ cells/mL and cultured overnight in DMEM containing 10% FBS and 1% Abs. The cells were washed twice with DPBS and treated with 5 μM TMR-labeled proteins in phenol red-free DMEM containing 1% FBS and 1% Abs for 2 h. The cells were washed twice with DPBS, supplemented with phenol red-free DMEM and imaged on a Nikon AIR live cell imaging confocal microscope. NIS Elements AR was used for image analysis.

### Flow Cytometry.

HeLa cells were seeded in a 24-well plate at a density 7.5×10⁴ cells/well. On the day of experiment, cells in DMEM media supplemented with 1% FBS and 1% Abs were incubated with 5 μM TMR-labeled proteins for 2 h. The cells were washed with cold DPBS and harvested by trypsinization. The detached cells were washed twice with DPBS, resuspended in DPBS, and analyzed by flow cytometry (BD FACS Aria III).

### Western Blotting.

NIH-3T3 cells were seeded in a 6-well plate at a density 10⁶ cells/well in standard DMEM supplemented with 10% FBS and 1% Abs at 37° C. in 5% CO₂. The cells were starved in serum free medium for 3 h. The cells were treated with different concentrations of MTD4-PTP1B for 4 h and stimulated with EGF (50 ng/mL) for 10 min. The cells were harvested, washed with PBS, and lysed in 100 μL of Pierce RIPA Buffer (Thermo) containing protease, phosphatase inhibitors and sodium pervanadate for 30 min on ice. The lysate was centrifuged at 15,000 rpm for 20 min. The total protein concentration of each sample was measured using the BCA Protein Assay Kit (Thermo). Equal amounts of protein were loaded onto each lane of a 10% SDS-PAGE gel (120 V, 2.5 h). The proteins were electrophoretically transferred to a nitrocellulose membrane at 4° C. (90 V, 2.5 h). The membrane was blocked with 5% BSA in TBST buffer (20 mM Tris pH 7.5, 150 mM NaCl, 0.10% (v/v) Tween-20) at room temperature for 1 h. Finally, the membrane was incubated with anti-pY antibody 4G10 (1:1000 dilution) at 4° C. overnight. The membrane was washed with TBST three times and incubated with fluorescently labeled secondary antibodies (1:10,000 dilution) for 2 h at room temperature. The membrane was washed three times again with TBST and signals were obtained using a LICOR Odyssey CLx machine.

For Western blotting with anti-p-Akt and anti-p-MEK antibodies. MiaPaCa-2 cells were seeded in 12-well plate at a density of 150,000 cells/well in DMEM media supplemented with 10% FBS and 1% ABS and incubated at 37° C., 5% CO₂. The following day, the cells were treated with PBS or varying concentrations of MTD4-RBDV or MTD4-NS1 for 4 h and then stimulated with EGF (50 ng/mL) for 10 min. Cells were harvested, lysed, and analyzed by Western blotting with anti-p-Akt and anti-p-MEK antibodies as described for MTD4-PTP1B.

HEK293T cells were seeded in a 6-well plate at a density 30×10⁵ cells/well in standard DMEM supplemented with 10% FBS and 1% Abs at 37° C. in 5% CO₂. The cells were treated with different concentrations of MTD4-PTP1B for 6 h in serum free media. The cells were harvested, washed with PBS, and lysed in 100 μL of Pierce RIPA Buffer (Thermo) containing protease, phosphatase inhibitors and sodium pervanadate for 30 min on ice. The lysate was centrifuged at 15,000 rpm for 20 min. The total protein concentration of each sample was measured using the BCA Protein Assay Kit (Thermo). Equal amounts of protein were loaded onto each lane of a 10% SDS-PAGE gel (120 V, 2.5 h). The proteins were electrophoretically transferred to a nitrocellulose membrane at 4° C. (90 V, 2.5 h). The membrane was blocked with 5% BSA in TBST buffer (20 mM Tris pH 7.5, 150 mM NaCl, 0.10% (v/v) Tween-20) at room temperature for 1 h. Finally, the membrane was incubated with anti-pY antibody 4G10 (1:1000 dilution) at 4° C. overnight. The membrane was washed with TBST three times and incubated with fluorescently labeled secondary antibodies (1:10,000 dilution) for 2 h at room temperature. The membrane was washed three times again with TBST and signals were obtained using a LICOR Odyssey CLx machine.

### Cell Viability Assay.

H358, MiaPaCa-2, H1915, or H1299 cells were seeded in white 96-well plates (5000 cells/well). The following day, cells were treated with a serially diluted protein solutions or PBS. Cells were incubated for 72 h at 37° C., 5% CO₂. After the addition of Cell Titer Glo and incubation for another 15 min, luminescence was measured on a TECAN instrument. Viability values reported are relative to that of PBS-treated control cells.

### BRET Assay.

HEK293T were seeded (650,000/well) in 6-well plate and incubated at 37° C., 5% CO₂. Next day, cells were transfected with pEF-RLUC8-L15-KrasG12D and pEF-CRAFRBD (1-149)-L15-GFP or pEF-RLUC8-L15-KrasG12V and pEF-CRAFRBD (1- 149)-L15-GFP in a 1:2 ratio (50 ng of KRAS and 100 ng of CRAFRBD-GFP). Cells were incubated for 24 h after transfection at 37° C. The following day, cells were seeded in a white 96-well plate (50,000 cells/well) and incubated for 4 h at 37° C., followed by the addition of varying concentrations of MTD4-RBDV or MTD4-NS1. After incubation for 20-24 h and the addition of 10 μM coelenterazine 400a substrate, the BRET signal was determined on a TECAN instrument.

### Annexin-V/PI Staining.

H358 cells were seeded into a 12-well microplate at a density of 10×10⁴ cells/well in 1 mL of RPMI containing 10% FBS and 1% Abs and incubated overnight at 37° C. with 5% CO₂. The following day, the medium was removed and the cells were washed with DPBS and treated with varying concentrations of MTD4-RBDV or MTD4-NS1 in 1 mL of DMEM media containing 10% FBS for 24 h at 37° C. with 5% CO₂. For cell harvesting, the medium was collected into a 15-mL falcon tube. The cells were washed with DPBS and the wash liquid was combined with the medium in the corresponding falcon tube. Adherent cells were treated with 250 μL of 0.25% trypsin/well for 3 min at 37° C. and transferred back to their respective falcon tubes. The cells were pelleted by centrifugation at 300 g, 4° C. for 5 min the cells were washed twice with DPBS to remove any remaining trypsin. Annexin V staining was next performed following Invitrogen's protocol. The cell pellets were resuspended in 100 μL of 1× annexin-binding buffer. Next, 5 μL of Alexa Fluor®488 Annexin V and 1 μL of propidium iodide (PI, 100 μg/ml) were added to the cell suspension and incubated at RT for 15 min. Finally, 400 μL of annexin-binding buffer was added to each tube immediately before analysis on a BD LSR Fortessa flow cytometer for fluorescence emission at 530 and 575 nm.

### Peptide Synthesis and Conjugation of HiBit Peptide.

All peptides were synthesized manually on rink amide resin using standard Fmoc chemistry. A typical coupling reaction contained 5 equiv of Fmoc-amino acid, 5 equiv of HATU, and 10 equiv of diisopropylethylamine (DIPEA) and was allowed to proceed with mixing for 30 min at room temperature (RT). For CPP12 with a single cysteine at the C-terminus, after the addition of the last (N-terminal) residue, the allyl group on the C-terminal Glu residue was removed by treatment with 0.3 equiv of Pd(PPh₃)₄ and 10 equiv of phenylsilane in anhydrous DCM in the dark (3×15 min). The resin was washed twice with sodium dimethyldithiocarbamate (SDDCM, 0.5 M in DMF) and the N-terminal Fmoc group was removed by treatment with 20% piperidine in DMF. The resin was extensively washed with DMF and DCM and incubated in 1 M 1-hydroxybenzotriazole (HOBt) in DMF for 20 min. The peptide was cyclized using 10 equiv of PyBOP, 10 equiv of HOBT, and 20 equiv of DIPEA in DMF for 1 h at RT. Cleavage and deprotection of the peptide were performed on resin using 92.5/2.5/2.5/2.5 (v/v) TFA/triisopropylsilane/1,3-dimethoxybenzene/water for 3 h at RT. HiBit peptide with a N-terminal cysteine (CVSGWRLFKKIS) (SEQ ID NO:152) was synthesized as described above. Purified HiBit peptide was activated with 2, 2′-dipyridyldisulfide (PyS-HiBit) and isolated. For conjugation, 1 equivalence of PyS-HiBit peptide was incubated with CPP12-Cys in methanol contain 2% acetic acid, the conjugated peptide was purified by reversed-phase HPLC on a semipreparative Waters XBridge C18 column. Similarly, 3 equiv. of PyS-HiBit was incubated with MTDs in absence of reducing agents for 3 h, the reaction mixture was passed through spin desalting column to remove excess HiBit peptide. A non-reducing SDS-PAGE gel was run to confirm the conjugation by monitoring a shift of ~1.3 kDa.

### HiBit Delivery Assay.

HEK293T cells were seeded in a 6-well plate at a density of 60×10⁴ cells/well in seeding media (DMEM supplemented with 10% FBS and 1% Abs). Next day, the cells were transfected with 0.5 μg of LgBit plasmid DNA using lipofectamine 2000 for 24 h. The cells were re-seeded in a 96 well plate at a density of 10,000 cells/well in seeding media overnight. The cells were treated with varying concentrations of conjugated or unconjugated HiBit peptide in media supplemented with 1% FBS and 1% Abs for 4 h. The cells were washed twice with DPBS before the addition of 100 μL of OPTIMEM and 25 μL of NanoLuc reagent per well. Luminescence was immediately measured using a Tecan Infinite M1000 Pro microplate reader. The values were normalized relative to that of untreated cells and plotted against the peptide concentration using GraphPad Prism software as mean±SD.

### GFP Complementation Assay.

HEK293T cells were seeded in a 6-well plate at a density of 60×10⁴ cells/well in media supplemented with 10% FBS and 1% Abs. Next day, the cells were transfected with 1 μg of GFP1-10 plasmid DNA using lipofectamine 2000 for 24 h. The cells were seeded in a 35/10 mm glass-bottomed microwell dish with four compartments at a density of 5×10⁴ cells/mL and cultured overnight in DMEM containing 10% FBS and 1% Abs. The cells were washed twice with DPBS and treated with DPBS (no treatment control) or 10 μM GFP11, CPP12-GFP11, or MTD4-GFP11 in media supplemented with 1% FBS and 1% Abs for 6 h. The cells were washed twice with DPBS, supplemented with phenol red free media, and imaged on Nikon AIR confocal microscope. NIS Elements AR was used for image analysis.

### Biodistribution Studies.

All animal experiments were performed in compliance with the institutional animal care guidelines and according to committee-approved protocols. Endotoxins were removed from the concentrated MENC protein using Pierce high-capacity endotoxin removal resin. Endotoxin-free MENC protein (8 mg/kg) was injected intravenously via tail vein into 4 floxed mice (transgenic mice with loxP modification). Two mice were sacrificed after 3 h while the other two after 48 h. Different organs were harvested, fixed in 4% paraformaldehyde for 24 h and transferred to 70% ethanol solution. The fixed samples were sent to iHisto Inc. for embedding, slicing and slide preparation. The slides were processed for imaging by washing them twice with 100% Xxylene, 100% ethanol, 95% ethanol and ddH₂O. The slides were stained with true black dye (Biotium), mounted and sealed with toluene. Slides were imaged on Nikon AIR confocal microscope and analyzed using NIS Elements AR software.

### Serum Stability Assay.

FN3 or MTD4 (10 μM) was incubated with 25% clarified human serum in a total volume of 100 μL. The mixture was incubated at 37° C. and 10-μL aliquots were withdrawn at different time points. The aliquots were immediately mixed with 10 μL of 2×SDS loading buffer, boiled for 5 min, and stored at −20° C. After incubation for up to 24 h, all aliquots were analyzed on a 15% SDS-PAGE gel and the gel was stained with Coomassie Blue. The gel was scanned on an Odyssey CLx Imager (LI-COR) in the 700-nm channel.

SEQUENCES

| SEQ ID NO | SEQUENCE |
|---|---|
| SEQ ID NO: 1 | FΦRRR |
| SEQ ID NO: 2 | FΦRRRC |
| SEQ ID NO: 3 | FΦRRRU |
| SEQ ID NO: 4 | RRRΦF |
| SEQ ID NO: 5 | RRRRΦF |
| SEQ ID NO: 6 | FΦRRRR |
| SEQ ID NO: 7 | FΦrRrR |
| SEQ ID NO: 8 | FΦrRrR |
| SEQ ID NO: 9 | FΦRRRR |
| SEQ ID NO: 10 | fΦRrRr |
| SEQ ID NO: 11 | RRFRΦR |
| SEQ ID NO: 12 | FRRRRΦ |
| SEQ ID NO: 13 | rRFRΦR |
| SEQ ID NO: 14 | RRΦFRR |
| SEQ ID NO: 15 | CRRRRFW |
| SEQ ID NO: 16 | FfΦRrRr |
| SEQ ID NO: 17 | FFΦRRRR |
| SEQ ID NO: 18 | RFRFRΦR |
| SEQ ID NO: 19 | URRRRFW |
| SEQ ID NO: 20 | CRRRRFW |
| SEQ ID NO: 21 | FΦRRRRQK |
| SEQ ID NO: 22 | FΦRRRRQC |
| SEQ ID NO: 23 | fΦRrRrR |
| SEQ ID NO: 24 | FΦRRRRR |
| SEQ ID NO: 25 | RRRRΦFDΩC |
| SEQ ID NO: 26 | FΦRRR |
| SEQ ID NO: 27 | FWRRR |
| SEQ ID NO: 28 | RRRΦF |
| SEQ ID NO: 29 | RRRWF |
| SEQ ID NO: 30 | FΦRRRR |
| SEQ ID NO: 31 | FFRRR |
| SEQ ID NO: 32 | FFrRr |
| SEQ ID NO: 33 | FFRrR |
| SEQ ID NO: 34 | FRFRR |
| SEQ ID NO: 35 | FRRFR |
| SEQ ID NO: 36 | FRRRF |
| SEQ ID NO: 37 | GΦRRR |
| SEQ ID NO: 38 | FFFRA |
| SEQ ID NO: 39 | FFFRR |
| SEQ ID NO: 40 | FFRRRR |
| SEQ ID NO: 41 | FRRFRR |
| SEQ ID NO: 42 | FRRRFR |
| SEQ ID NO: 43 | RFFRRR |
| SEQ ID NO: 44 | RFRRFR |
| SEQ ID NO: 45 | FRFRRR |
| SEQ ID NO: 46 | FFFRRR |
| SEQ ID NO: 47 | FFRRRF |
| SEQ ID NO: 48 | FRFFRR |
| SEQ ID NO: 49 | RRFFFR |
| SEQ ID NO: 50 | FFRFRR |
| SEQ ID NO: 51 | FFRRFR |
| SEQ ID NO: 52 | FRRFFR |
| SEQ ID NO: 53 | FRRFRF |
| SEQ ID NO: 54 | FRFRFR |
| SEQ ID NO: 55 | RFFRFR |
| SEQ ID NO: 56 | GΦRRRR |
| SEQ ID NO: 57 | FFFRRRR |
| SEQ ID NO: 58 | RFFRRRR |
| SEQ ID NO: 59 | RRFFRRR |
| SEQ ID NO: 60 | RFFFRRR |
| SEQ ID NO: 61 | RRFFFRR |
| SEQ ID NO: 62 | FFRRFRR |
| SEQ ID NO: 63 | FFRRRRF |
| SEQ ID NO: 64 | FRRFFRR |
| SEQ ID NO: 65 | FFFRRRRR |
| SEQ ID NO: 66 | FFFRRRRRR |
| SEQ ID NO: 67 | FΦRrRr |
| SEQ ID NO: 68 | XXRRRR |
| SEQ ID NO: 69 | FfFRrR |
| SEQ ID NO: 70 | fFfrRr |
| SEQ ID NO: 71 | fFfRrR |
| SEQ ID NO: 72 | FfFrRr |
| SEQ ID NO: 73 | fFΦrRr |
| SEQ ID NO: 74 | fΦfrRr |
| SEQ ID NO: 75 | ΦFfrRr |
| SEQ ID NO: 76 | FΦrRr |
| SEQ ID NO: 77 | fΦrRr |
| SEQ ID NO: 78 | Ac-Lys-fFRrRrD |
| SEQ ID NO: 79 | Ac-Dap-fFRrRrD |
| SEQ ID NO: 80 | Pip-Nal-Arg-Glu-arg-arg-glu |
| SEQ ID NO: 81 | Pip-Nal-Arg-Arg-arg-arg-glu |
| SEQ ID NO: 82 | Pip-Nal-Nal-Arg-arg-arg-glu |
| SEQ ID NO: 83 | Pip-Nal-Nal-Arg-arg-arg-Glu |
| SEQ ID NO: 84 | Pip-Nal-Phe-Arg-arg-arg-glu |
| SEQ ID NO: 85 | Pip-Nal-Phe-Arg-arg-arg-Glu |
| SEQ ID NO: 86 | Pip-Nal-phe-Arg-arg-arg-glu |
| SEQ ID NO: 87 | Pip-Nal-phe-Arg-arg-arg-Glu |
| SEQ ID NO: 88 | Pip-Nal-nal-Arg-arg-arg-Glu |
| SEQ ID NO: 89 | Pip-Nal-nal-Arg-arg-arg-glu |
| SEQ ID NO: 90 | [P-Cha-r-Cha-r-Cha-r-Cha-r-G]^{d} |
| SEQ ID NO: 91 | Tm(SvP-F₂Pmp-H)-Dap-(FΦRRRR-Dap)]^{f} |
| SEQ ID NO: 92 | [Tm(a-Sar-D-pThr-Pip-ΦRAa)-Dap-(FΦRRRR-Dap)]^{f} |
| SEQ ID NO: 93 | [CRRSRRGCGRRSRRCG]^{g} |
| SEQ ID NO: 94 | [K(Dod)RRRR] |
| SEQ ID NO: 95 | LKKLCKLLKKLCKLAG |
| SEQ ID NO: 96 | RRRR-[KRRRE]^{c} |
| SEQ ID NO: 97 | RRR-[KRRRRE]^{c} |
| SEQ ID NO: 98 | RR-[KRRRRRE]^{c} |
| SEQ ID NO: 99 | R-[KRRRRRRE]^{c} |
| SEQ ID NO: 100 | [CRCRCRCR] |
| SEQ ID NO: 101 | [Pra-LRKRLRKFRN-AzK]^{Q} |
| SEQ ID NO: 102 | T-Dap-[Dap-Dap-f-L-Dap-Dap-T] |
| SEQ ID NO: 103 | T-Agp-[Dap-Agp-f-L-Agp-Agp-T] |
| SEQ ID NO: 104 | RRRWWW |
| SEQ ID NO: 105 | WWWRRR |
| SEQ ID NO: 106 | GDSPAS |
| SEQ ID NO: 107 | GGSGGSGGS |
| SEQ ID NO: 108 | TGRGDSPASSKPI |
| SEQ ID NO: 109 | APAVTVRYY |
| SEQ ID NO: 110 | GGNSPVQE |
| SEQ ID NO: 111 | TGRRRRWWWSKPI |
| SEQ ID NO:112 | APWWWRRRRYY |
| SEQ ID NO: 113 | GGRRRRWWWVQE |
| SEQ ID NO: 114 | APAWYWRYY |
| SEQ ID NO: 115 | TGRRRRSKPI |
| SEQ ID NO: 116 | APARRRRYY |
| SEQ ID NO: 117 | TGWYWRSKPI |
| SEQ ID NO: 118 | MGSSHHHHHHSSGLVPRGSHMVSDVPRDLEVVAATPTSLLISWDAPAVTVRYYRITYGET |
| | GGNSPVQEFTVPGSKSTATISGLKPGVDYTITVYAVTGRGDSPASSKPISINYRTGGSGGS |
| | GGSELC |
| SEQ ID NO: 119 | MGSSHHHHHHSSGLVPRGSHMVSDVPRDLEVVAATPTSLLISWDAPAVTVRYYRITYGET |
| | GGNSPVQEFTVPGSKSTATISGLKPGVDYTITVYAVTGRRRRWWWSKPISINYRTGGSGG |
| | SGGSELC |
| SEQ ID NO: 120 | MGSSHHHHHHSSGLVPRGSHMVSDVPRDLEVVAATPTSLLISWDAPWWWRRRRYYRIT |
| | YGETGGNSPVQEFTVPGSKSTATISGLKPGVDYTITVYAVTGRGDSPASSKPISINYRTGG |
| | SGGSGGSELC |
| SEQ ID NO: 121 | MGSSHHHHHHSSGLVPRGSHMVSDVPRDLEVVAATPTSLLISWDAPAVTVRYYRITYGET |
| | GGRRRRWWWVQEFTVPGSKSTATISGLKPGVDYTITVYAVTGRGDSPASSKPISINYRTG |
| | GSGGSGGSELC |
| SEQ ID NO: 122 | MGSSHHHHHHSSGLVPRGSHMVSDVPRDLEVVAATPTSLLISWDAPAWYWRYYRITYGE |
| | TGGNSPVQEFTVPGSKSTATISGLKPGVDYTITVYAVTGRRRRSKPISINYRTGGSGGSGG |
| | SELC |
| SEQ ID NO: 123 | MGSSHHHHHHSSGLVPRGSHMVSDVPRDLEVVAATPTSLLISWDAPAWYWRYYRITYGE |
| | TGGNSPVQEFTVPGSKSTATISGLKPGVDYTITVYAVTGRGDSPASSKPISINYRTGGSGG |
| | SGGSELC |
| SEQ ID NO: 124 | MGSSHHHHHHSSGLVPRGSHMVSDVPRDLEVVAATPTSLLISWDAPAVTVRYYRITYGET |
| | GGNSPVQEFTVPGSKSTATISGLKPGVDYTITVYAVTGRRRRSKPISINYRTGGSGGSGG |
| | SELC |
| SEQ ID NO: 125 | MGSSHHHHHHSSGLVPRGSHMVSDVPRDLEVVAATPTSLLISWDAPARRRRYYRITYGET |
| | GGNSPVQEFTVPGSKSTATISGLKPGVDYTITVYAVTGWYWRSKPISINYRTGGSGGSGGSELC |
| SEQ ID NO: 126 | MGSSHHHHHHSSGLVPRGSHMVSDVPRDLEVVAATPTSLLISWDAPAWYWRYYRITYGE |
| | TGGNSPVQEFTVPGSKSTATISGLKPGVDYTITVYAVTGRRRRSKPISINYRTGGSGGSGG |
| | SELMEMEKEFEQIDKSGSWAAIYQDIRHEASDFPCRVAKLPKNKNRNRYRDVSPFDHSRIK |
| | LHQEDNDYINASLIKMEEAQRSYILTQGPLPNTCGHFWEMVWEQKSRGVVMLNRVMEKGS |
| | LKCAQYWPQKEEKEMIFEDTNLKLTLISEDIKSYYTVRQLELENLTTQETREILHFHYTTWPD |
| | FGVPESPASFLNFLFKVRESGSLSPEHGPVVVHCSAGIGRSGTFCLADTCLLLMDKRKDPS |
| | SVDIKKVLLEMRKFRMGLIQTADQLRFSYLAVIEGAKFIMGDSSVQDQWKELSHEDLEPPPE |
| | HIPPPPRPPKRILEPHN |
| SEQ ID NO: 127 | MGSSHHHHHHSSGLVPRGSHMVSDVPRDLEVVAATPTSLLISWDAPAWYWRYYRITYGE |
| | TGGNSPVQEFTVPGSKSTATISGLKPGVDYTITVYAVTGRRRRSKPISINYRTGGSGGSGG |
| | SELGSVSSVPTKLEVVAATPTSLLISWDAPAVTVDYYVITYGETGGNSPVQKFEVPGSKST |
| | ATISGLKPGVDYTITVYAWGWHGQVYYYMGSPISINYRT |
| SEQ ID NO: 128 | MGSSHHHHHHSSGLVPRGSHMVSDVPRDLEVVAATPTSLLISWDAPAWYWRYYRITYGE |
| | TGGNSPVQEFTVPGSKSTATISGLKPGVDYTITVYAVTGRRRRSKPISINYRTGGSGGSGG |
| | SELKTSNTIRVLLPNQEWTVVKVRNGMSLHDSLMKALKRHGLQPESSAVFRLLHEHKGKK |
| | ARLDWNTDAASLIGEELQVDFLDHVPLTTHNFARKTFLKLGIHRD |
| SEQ ID NO: 129 | AVTVR |
| SEQ ID NO: 130 | GGNSPVQ |
| SEQ ID NO: 131 | PGSK |
| SEQ ID NO: 132 | GRGDSPAS |
| SEQ ID NO: 133 | RRRRWWWW |
| SEQ ID NO: 134 | APAYWWRYY |
| SEQ ID NO: 135 | TGRRRRSKPI |
| SEQ ID NO: 136 | APAWWARYY |
| SEQ ID NO: 137 | TGRRRRSKPI |
| SEQ ID NO: 138 | APAWWWRRYY |
| SEQ ID NO: 139 | TGRRASSKPI |
| SEQ ID NO: 140 | APAWWARYY |
| SEQ ID NO: 141 | TGWRRRRSKPI |
| SEQ ID NO: 142 | APAWWRRYY |
| SEQ ID NO: 143 | MGSSHHHHHHSSGLVPRGSHMVSDVPRDLEVVAATPTSLLISWDAPAYWWRYYRITYG |
| | ETGGNSPVQEFTVPGSKSTATISGLKPGVDYTITVYAVTGRRRRSKPISINYRTGGSGGS |
| | GGSELC |
| SEQ ID NO: 144 | MGSSHHHHHHSSGLVPRGSHMVSDVPRDLEVVAATPTSLLISWDAPAWWARYYRITYG |
| | ETGGNSPVQEFTVPGSKSTATISGLKPGVDYTITVYAVTGRRRRSKPISINYRTGGSGGS |
| | GGSELC |
| SEQ ID NO: 145 | MGSSHHHHHHSSGLVPRGSHMVSDVPRDLEVVAATPTSLLISWDAPAWWWRRYYRITY |
| | GETGGNSPVQEFTVPGSKSTATISGLKPGVDYTITVYAVTGRRASSKPISINYRTGGSGG |
| | SGGSELC |
| SEQ ID NO: 146 | MGSSHHHHHHSSGLVPRGSHMVSDVPRDLEVVAATPTSLLISWDAPAWWARYYRITYG |
| | ETGGNSPVQEFTVPGSKSTATISGLKPGVDYTITVYAVTGWRRRRSKPISINYRTGGSGG |
| | SGGSELC |
| SEQ ID NO: 147 | MGSSHHHHHHSSGLVPRGSHMVSDVPRDLEVVAATPTSLLISWDAPAWWRRYYRITYG |
| | ETGGNSPVQEFTVPGSKSTATISGLKPGVDYTITVYAVTGRRWWSKPISINYRTGGSGGS |
| | GGSELC |
| SEQ ID NO: 148 | MGSSHHHHHHSSGLVPRGSHMVSDVPRDLEVVAATPTSLLISWDAPAWYWRYYRITYG |
| | ETGGNSPVQEFTVPGSKSTATISGLKPGVDYTITVYAVTGRRRRSKPISINYRTGGSGGS |
| | GGSELSKGEELFTGVVPILVELDGDVNGHKFSVSGEGEGDATYGKLTLKFICTTGKLPVP |
| | WPTLVTTLTYGVQCFSRYPDHMKRHDFFKSAMPEGYVQERTIFFKDDGNYKTRAEVKFE |
| | GDTLVNRIELKGIDFKEDGNILGHKLEYNYNDHQVYIMADKQKNGIKANFKIRHNIEDGGV |
| | QLADHYQQNTPIGDGPVLLPDNHYLFTTSTLSKDPNEKRDHMVLLEFVTAAGITHGMDEL |
| | YK |
| SEQ ID NO: 149 | MGSSHHHHHHSSGLVPRGSHMVSDVPRDLEVVAATPTSLLISWDAPAWYWRYYRITYG |
| | ETGGNSPVQEFTVPGSKSTATISGLKPGVDYTITVYAVTGRRRRSKPISINYRTGGSGGS |
| | GGSELMVSKGEELFTGVVPILVELDGDVNGHKFSVSGEGEGDATYGKLTLKFICTTGKLP |
| | VPWPTLVTTLTYGVQCFSRYPDHMKQHDFFKSAMPEGYVQERTIFFKDDGNYKTRAEVK |
| | FEGDTLVNRIELKGIDFKEDGNILGHKLEYNYNSHNVYIMADKQKNGIKVNFKIRHNIEDGS |
| | VQLADHYQQNTPIGDGPVLLPDNHYLSTQSALSKDPNEKRDHMVLLEFVTAAGITLGMDE |
| | LYKSGEQKLISEEDLGGPKKKRKVSNLLTVHQNLPALPVDATSDEVRKNLMDMFRDRQA |
| | FSEHTWKMLLSVCRSWAAWCKLNNRKWFPAEPEDVRDYLLYLQARGLAVKTIQQHLGQ |
| | LNMLHRRSGLPRPSDSNAVSLVMRRIRKENVDAGERAKQALAFERTDFDQVRSLMENS |
| | DRCQDIRNLAFLGIAYNTLLRIAEIARIRVKDISRTDGGRMLIHIGRTKTLVSTAGVEKALSL |
| | GVTKLVERWISVSGVADDPNNYLFCRVRKNGVAAPSATSQLSTRALEGIFEATHRLIYGA |
| | KDDSGQRYLAWSGHSARVGAARDMARAGVSIPEIMQAGGWTNVNIVMNYIRNLDSETG |
| | AMVRLLEDGD |

1. A peptide, comprising: a membrane translocation domain having one or more cell penetrating peptide motifs, where at least one cell penetrating peptide motif is from 3 to 10 amino acid residues in length and has at least three arginine and/or lysine residues; or where at least one cell penetrating peptide motif is from 3 to 10 amino acid residues in length and has at least two arginine and/or lysine residues and at least one other cell penetrating peptide motif is from 2 to 8 amino acid residues in length and has at least two hydrophobic residues.

2. The peptide of claim 1, wherein the membrane translocation domain is a mammalian membrane translocation domain.

3. The peptide of claim 1, wherein the membrane translocation domain is human fibronectin type III.

4. The peptide of claim 1, wherein the human fibronectin type III has 90% sequence similarity with SEQ. ID. NO.118.

5. The peptide of claim 1, wherein the cell penetrating motif has from 3 to 10 adjacent arginine residues.

6. The peptide of claim 1, wherein the membrane translocation domain is human fibronectin type III having BC, DE, CD, and FG loops and one or more of the BC, DE, CD, or FG loops have cell penetrating peptide motifs.

7. The peptide of claim 1, wherein the membrane translocation domain is human fibronectin type III having BC, DE, CD, and FG loops and two of the BC, DE, CD, or FG loops have cell penetrating peptide motifs.

8. The peptide of claim 1, wherein the membrane translocation domain is human fibronectin type III having BC, DE, CD, and FG loops and the BC and either the DE, CD, or FG loops have cell penetrating peptide motifs.

9. The peptide of claim 1, wherein the membrane translocation domain is human fibronectin type III having BC, DE, CD, and FG loops and the BC and FG loops have cell penetrating peptide motifs.

10. The peptide of claim 9, wherein the cell penetrating peptide motif in the BC loop has from 2 to 8 amino acid residues and has at least two hydrophobic amino acid residues and the cell penetrating peptide motif in the FG loop has from 3 to 10 amino acid residues and has at least two adjacent arginine and/or lysine residues.

11. The peptide of claim 9, wherein the cell penetrating peptide motif in the FG loop has from 2 to 8 amino acid residues and has at least two hydrophobic amino acid residues and the cell penetrating peptide motif in the BC loop has from 3 to 10 amino acid residues and has at least two adjacent arginine and/or lysine residues.

12. The peptide of claim 1, wherein a second cell penetrating peptide motif is present and is WW, FF, WF, FW, WWW, FFF, WFW, FWF, WWF, WFF, FWW, FFW, WYW, WWH, YWW, or WYH.

13. The peptide of claim 1, wherein the cell penetrating peptide motif is RRRWWW (SEQ. ID. NO.:104) or WWWRRR (SEQ. ID. NO.:105).

14. The peptide of claim 1, comprising
(SEQ. ID. NO.: 111)
TGRRRRWWWSKPI;
(SEQ. ID. NO.: 112)
APWWWRRRRYY;
(SEQ. ID. NO.: 113)
GGRRRRWWWVQE;
(SEQ. ID. NO.: 114)
APAWYWRYY;
(SEQ. ID. NO.: 115)
TGRRRRSKPI;
(SEQ. ID. NO.: 116)
APARRRRYY;
or
(SEQ. ID. NO.: 117)
TGWYWRSKPI

15. The peptide of claim 1, comprising SEQ. ID. NO.s: 119, 120, 121, 122, 123, 124, or 125.

16. The peptide of claim 1, further comprising a cargo moiety linked to the membrane translocation domain.

17. The peptide of claim 16, wherein the cargo moiety is linked to the membrane translocation domain at a N-terminus or C-terminus of the membrane translocation domain, or at a side chain within the membrane translation domain.

18. The peptide of claim 16, wherein the cargo moiety is selected from the group consisting of a detectable moiety, a targeting moiety, a therapeutic moiety, or any combination thereof.

19. The peptide of claim 16, comprising SEQ. ID. NO.: 126, 127, or 128.

20. A composition for delivering a cargo moiety into a cell comprising SEQ. ID. NO.:122 covalently bound to a cargo, wherein the cargo moiety is selected from the group consisting of a detectable moiety, a targeting moiety, a therapeutic moiety, or any combination thereof.

21. A method of delivering a cargo moiety into a cell, comprising contacting the cell with the peptide of claim 20.

22. A method of delivering an agricultural product into a plant cell, comprising contacting the cell with a peptide of claim 20.
